SEQUENCE LISTING (3 sequences)

SEQ ID NO: 1 - DNA, 1440 bp, Artificial Sequence
  chimeric antigen receptor
atggctctgcctgtgacagctcttctgcttcctctggccctgctgctgcacgctgctagaccagacatccagatgacccagaccaccagcagcctgagcgcttctctgggcgatagagtcaccatcagctgcagagccagccaggacatcagcaagtatctgaactggtaccagcagaagcccgacggcaccgtcaagctgctcatctaccacacctctcggctgcacagcggcgtcccttctcgattctctggctctggaagcggcacagactacagcctcaccatctccaacctggagcaggaggacatcgccacctacttctgccagcagggcaacaccctgccttacaccttcggcggaggaacaaagctggagatcaccggcagcacaagcggcagcggcaagcctggatctggcgaaggctctacaaagggcgaggtcaaattacaagagagcggccctggcctggtggccccttctcagtctttatctgtgacatgcaccgtgtccggcgtgtcccttcccgactatggcgtttcctggatcagacagccccctcggaagggtttggaatggctgggcgtcatttggggcagcgagaccacttactacaacagcgccctgaagtcacggctcacaatcatcaaggacaacagcaagagccaggtgttcctgaagatgaacagcctgcagaccgacgacaccgccatctactactgcgccaagcactactactacggcggctcttatgcaatggactactggggccagggcaccagcgtcacagtgtcttcagctgctgctctggacaacgagaagagcaacggcaccatcattcatgtgaagggcaagcacctgtgccccagccccttattccccggaccttctaagcccttctgggtcctggttgtggtgggcggcgtgttagcttgctactccctgctggtgacagtcgccttcatcatcttttgggtcagaagcaagagaagcagactgctccacagcgactacatgaacatgaccccccggagacctggccctaccagaaagcattaccagccctacgccccacccagagacttcgccgcatatagatcacgtgttaagtttagcaggagcgccgacgcgcctgcctaccagcaaggccaaaatcagctttacaacgagctgaacctgggcagaagagaggagtatgacgtcctggacaagagacggggcagagaccccgagatgggcggaaaacctagaagaaagaacccgcaggagggcctgtacaatgagctgcaaaaggacaagatggccgaggcctacagcgagatcggcatgaagggcgaaagaagaagaggcaagggccacgacggcctgtatcagggccttagcacagccaccaaggacacctacgacgctctgcacatgcaggccctgcctccgagatga

SEQ ID NO: 2 - protein, 18 aa, Artificial Sequence
  linker peptide
GSTSGSGKPGSGEGSTKG

SEQ ID NO: 3 - protein, 4 aa, Artificial Sequence
  peptide linker
GGGS

(19) United States
(12) Patent Application Publication
(10) Pub. No.: US 2021/0040449 A1
(43) Pub. Date: Feb. 11, 2021

(54) MODIFIED PLURIPOTENT STEM CELLS AND METHODS OF MAKING AND USE

(71) Applicant: KITE PHARMA, INC., Santa Monica, CA (US)

(72) Inventors: Eric GSCHWENG, Millbrae, CA (US); Rajul JAIN, Santa Monica, CA (US); Yong OUYANG, Santa Monica, CA (US); Arianne PEREZ GARCIA, Santa Monica, CA (US); Margo ROBERTS, Santa Monica, CA (US); Ruben ALVAREZ RODRIGUEZ, Santa Monica, CA (US); Drake SMITH, Santa Monica, CA (US); Xingliang ZHOU, Santa Monica, CA (US)

(21) Appl. No.: 16/969,127

(22) PCT Filed: Feb. 15, 2019
(86) PCT No.: PCT/US19/18310

§ 371 Date: Aug. 11, 2020

Related U.S. Application Data
(60) Provisional application No. 62/673,624, filed on May 18, 2018.
(60) Provisional application No. 62/710,591, filed on Feb. 16, 2018.

(51) Int. Cl. C12N 5/0783; C12N 15/10; C07K 14/725; C07K 14/74; C12N 5/074

(52) U.S. Cl. CPC C12N 5/0636; C12N 15/102; C07K 14/7051; C12N 2510/00; C12N 5/0696; C12N 2506/03; C07K 14/70539

(57) ABSTRACT

The disclosure provides a method of generating modified T cells from engineered stem cells for use in an autologous or allogeneic setting for engineered immunotherapy. The knockout of endogenous TCR or HLA expression allows for engineering of modified pluripotent stem cells that reduce or eliminate the risk of Graft versus Host Disease (GVHD), provide resistance to elimination by a recipient's T cells and NK cells, and allow for controllable T cell activity. Thus, this method allows the development of T cells with reduced immune reactivity.

### CROSS-REFERENCE TO RELATED APPLICATIONS

This application claims priority to U.S. Provisional Application No. 62/710,591, filed Feb. 16, 2018, and to U.S. Provisional Application No. 62/673,624, filed May 18, 2018, both of which are incorporated by reference herein in their entirety.

### SEQUENCE LISTING

The instant application contains a Sequence Listing which has been submitted electronically in ASCII format and is hereby incorporated by reference in its entirety. Said ASCII copy, created on Feb. 14, 2019, is named K-1061_01_SL.txt and is 2.67 kilobytes in size.

### BACKGROUND

Human cancers are by their nature comprised of normal cells that have undergone a genetic or epigenetic conversion to become abnormal cancer cells. In doing so, cancer cells begin to express proteins and other antigens that are distinct from those expressed by normal cells. These aberrant tumor antigens may be used by the body's innate immune system to specifically target and kill cancer cells. However, cancer cells employ various mechanisms to prevent immune cells, such as T and B lymphocytes, from successfully targeting cancer cells.

Current T cell therapies rely on enriched or modified human T cells to target and kill cancer cells in a patient. To increase the ability of T cells to target and kill a particular cancer cell, methods have been developed to engineer T cells to express constructs, which direct T cells to a particular target cancer cell. Chimeric antigen receptors (CARs) and engineered T cell receptors (TCRs), which comprise binding domains capable of interacting with a particular tumor antigen, allow T cells to target and kill cancer cells that express the particular tumor antigen.

A need exists for improved methods of generating CARs, TCRs, and antigen receptor modified T cells for specifically targeting and killing cancer cells.

### SUMMARY

The present disclosure addresses this need by, among other things, providing compositions and methods comprising genetically engineered stem cells and their derivatives that efficiently differentiate into T cells. In particular, the present disclosure provides the production of stem cells which may be used in an autologous or allogeneic setting for engineered immunotherapy. When used in cell based immunotherapy, modified pluripotent stem cells described herein may reduce or eliminate the risk of Graft versus Host Disease (GVHD), provide resistance to elimination by a recipient's T cells and NK cells, and allow for controllable T cell activity (e.g., engineered to comprise a suicide gene or kill switch).

T cell responses from adoptive cell therapy may be mediated by T-cells from the recipient. Graft rejection may arise from immunogenicity to the exogenous transgene, reactivity against mismatched Human Histocompatibility Antigen (HLA) (unrelated/haploidentical), or reactivity against minor histocompatibility antigens (MiHA) (e.g., HA-1, HA-2, etc.) (related/unrelated HLA matched/haploidentical). Responses may also be mediated by the donor T-cells leading to GVHD from reactivity against mismatched HLA/MiHA and anti-tumor events from reactivity against tumor antigens/tumor associated MiHA.

To prevent host immune reactivity to cell therapy, (e.g., GVHD induced by mismatched HLA or MiHA), in one aspect, the present disclosure provides a modified pluripotent stem cell engineered to eliminate endogenous TCR expression. In some embodiments, gene editing of endogenous TCR is engineered by knock out (KO) of TCRα and/or TCRβ (TRAC and/or TRBC1/TRBC2). In some embodiments, cells are engineered by KO of RAG1/RAG2 (depending on cell source and differentiation status).

To prevent graft rejection, the present disclosure provides a modified pluripotent stem cell engineered to block expression of donor HLA and/or re-introduce 1 HLA-Class I “non-polymorphic” allele to prevent NK killing (e.g., single chain HLA-E). In some embodiments, modifications are made to HLA Class I molecules (e.g., B-2-microglobulin, individual HLA molecules (HLA-A,-B,-C,-E,-G), TAP1, TAP2 and/or genes associated with Bare Lymphocyte Syndrome I (BLSI)). In some embodiments, modifications are made to HLA Class II molecules (e.g., Transcription factors (RFXANK or RFX5 or RFXAP) or transactivators (CIITA), Genes associated with BLS II, and/or individual HLA molecules (HLA-DP,-DQ-DR,-DM,-DO -alpha and beta chains)). In some embodiments, modifications are made to promote tumor reactivity (e.g., introducing a tumor specific TCR or CAR). In some embodiments, cells are further modified to eliminate inhibitory receptors (e.g., PDCD1, CTLA4). In some embodiments, cells are modified to introduce costimulatory receptors (e.g., CD28, TNFRSF9).

In one aspect, the present disclosure provides a modified pluripotent stem cell engineered to eliminate endogenous TCR or HLA expression.

In some embodiments, the modified pluripotent stem cell comprises a deficient TCRα constant (TRAC) gene, a deficient TCRβ constant (TRBC) gene or a deficient beta 2 microglobulin (b2m) gene, optionally wherein the deficient gene is created by knockout. In some embodiments, the modified pluripotent stem cell comprises a deficient TCRα constant (TRAC) gene.

In some embodiments, the modified pluripotent stem cell comprises a deficient TCRβ constant (TRBC) gene.

In some embodiments, the modified pluripotent stem cell comprises a deficient beta 2 microglobulin (b2m) gene.

In some embodiments, the deficient gene is created by knockout.

In some embodiments, the deficient gene is edited using CRISPR/Cas9, a zinc finger nuclease (ZFN), a TALEN, a MegaTAL, a meganuclease, Cpf1, homologous recombination, or a single stranded oligodeoxynucleotide (ssODN). In some embodiments, the deficient gene is edited using a zinc finger nuclease (ZFN).

In some embodiments, the cell comprises an exogenous construct encoding a single chain HLA trimer, a single chain HLA trimer comprising an HLA linked to beta-2-microglobulin linked to a stabilizing peptide, optionally, wherein the HLA trimer is HLA-E, HLA-G, or a combination of HLA-E and HLA-G; an exogenous construct encoding a chimeric antigen receptor (CAR) that targets a tumor antigen, optionally, wherein the tumor antigen is selected from a tumor-associated surface antigen, such as 5T4, alphafetoprotein (AFP), B7-1 (CD80), B7-2 (CD86), BCMA, B-human chorionic gonadotropin, CA-125, carcinoembryonic antigen (CEA), carcinoembryonic antigen (CEA), CD123, CD133, CD138, CD19, CD20, CD22, CD23, CD24, CD25, CD30, CD33, CD34, CD4, CD40, CD44, CD56, CD70, CD8, CLL-1, c-Met, CMV-specific antigen, CS-1, CSPG4, CTLA-4, DLL3, disialoganglioside GD2, ductal-epithelial mucine, EBV-specific antigen, EGFR variant III (EGFRvIII), ELF2M, endoglin, ephrin B2, epidermal growth factor receptor (EGFR), epithelial cell adhesion molecule (EpCAM), epithelial tumor antigen, ErbB2 (HER2/neu), fibroblast associated protein (fap), FLT3, folate binding protein, GD2, GD3, glioma-associated antigen, glycosphingolipids, gp36, HBV-specific antigen, HCV-specific antigen, HER1-HER2, HER2-HER3 in combination, HERV-K, high molecular weight-melanoma associated antigen (HMW-MAA), HIV-1 envelope glycoprotein gp41, HPV-specific antigen, human telomerase reverse transcriptase, IGFI receptor, IGF-II, IL-11Ralpha, IL-13R-a2, Influenza Virus-specific antigen; CD38, insulin growth factor (IGFl)-l, intestinal carboxyl esterase, kappa chain, LAGA-la, lambda chain, Lassa Virus-specific antigen, lectin-reactive AFP, lineage-specific or tissue specific antigen such as CD3, MAGE, MAGE-A1, major histocompatibility complex (MHC) molecule, major histocompatibility complex (MHC) molecule presenting a tumor-specific peptide epitope, M-CSF, melanoma-associated antigen, mesothelin, mesothelin, MN-CA IX, MUC-1, mut hsp70-2, mutated p53, mutated p53, mutated ras, neutrophil elastase, NKG2D, Nkp30, NY-ESO-1, p53, PAP, prostate, prostate specific antigen (PSA), prostate-carcinoma tumor antigen-1 (PCTA-1), prostate-specific antigen protein, STEAP1, STEAP2, PSMA, RAGE-1, ROR1, RU1, RU2 (AS), surface adhesion molecule, surviving and telomerase, TAG-72, the extra domain A (EDA) and extra domain B (EDB) of fibronectin and the Al domain of tenascin-C (TnC Al), thyroglobulin, tumor stromal antigens, vascular endothelial growth factor receptor-2 (VEGFR2), virus-specific surface antigen such as an HIV-specific antigen (such as HIV gpl20), as well as any derivate or variant of these surface markers; an exogenous construct encoding a TCR, optionally, wherein the TCR is an alpha/beta TCR, gamma/delta TCR, a cancer or cancer associated antigen reactive TCR, a TCR that is reactive against murine or other non-human MHC, a class I or class II restricted TCR, an HPV recognizing TCR, a viral reactive TCR, an EBV TCR, a CMV TCR, or an influenza TCR, an HPV-16 E6 TCR, HPV-16 E7 TCR, or MAGEA3/A6 TCR or engineered variant, or TCR is derived from CD8, CD4, CD4/8 double positive, immature or developing T cell, Treg, NKT, or NK T cell; and/or an exogenous construct encoding a suicide gene, wherein the suicide gene allows for the elimination of gene modified cells or is used as a PET reporter gene for non-invasive imaging, optionally, wherein the suicide gene is sr39TK, is a chemically induced caspase, dimerization induced by a small molecule/chemically induced dimerizer (CID), a selectable surface marker, or a selectable surface marker selected from CD19, CD20, CD34, EGFR or LNGFR.

In some embodiments, the modified pluripotent stem cell comprises an exogenous construct encoding a single chain HLA trimer. In some embodiments, the single chain HLA trimer comprises an HLA class I HLA-E. In some embodiments, the single chain HLA trimer comprises an HLA linked to beta-2-microglobulin linked to a stabilizing peptide. In some embodiments, the HLA trimer is HLA-E, HLA-G, or a combination of HLA-E and HLA-G.

In some embodiments, the modified pluripotent stem cell comprises an exogenous construct encoding a chimeric antigen receptor (CAR). In some embodiments, the CAR targets a tumor antigen. In some embodiments, the tumor antigen is selected from a tumor-associated surface antigen, such as 5T4, alphafetoprotein (AFP), B7-1 (CD80), B7-2 (CD86), BCMA, B-human chorionic gonadotropin, CA-125, carcinoembryonic antigen (CEA), carcinoembryonic antigen (CEA), CD123, CD133, CD138, CD19, CD20, CD22, CD23, CD24, CD25, CD30, CD33, CD34, CD4, CD40, CD44, CD56, CD8, CLL-1, c-Met, CMV-specific antigen, CS-1, CSPG4, CTLA-4, DLL3, disialoganglioside GD2, ductal-epithelial mucine, EBV-specific antigen, EGFR valiant III (EGFRvIII), ELF2M, endoglin, ephrin B2, epidermal growth factor receptor (EGFR), epithelial cell adhesion molecule (EpCAM), epithelial tumor antigen, ErbB2 (HER2/neu), fibroblast associated protein (fap), FLT3, folate binding protein, GD2, GD3, glioma-associated antigen, glycosphingolipids, gp36, HBV-specific antigen, HCV-specific antigen, HER1-HER2, HER2-HER3 in combination, HERV-K, high molecular weight-melanoma associated antigen (HMW-MAA), HIV-1 envelope glycoprotein gp41, HPV-specific antigen, human telomerase reverse transcriptase, IGFI receptor, IGF-II, IL-11Ralpha, IL-13R-a2, Influenza Virus-specific antigen; CD38, insulin growth factor (IGFl)-l, intestinal carboxyl esterase, kappa chain, LAGA-la, lambda chain, Lassa Virus-specific antigen, lectin-reactive AFP, lineage-specific or tissue specific antigen such as CD3, MAGE, MAGE-A1, major histocompatibility complex (MHC) molecule, major histocompatibility complex (MHC) molecule presenting a tumor-specific peptide epitope, M-CSF, melanoma-associated antigen, mesothelin, mesothelin, MN-CA IX, MUC-1, mut hsp70-2, mutated p53, mutated p53, mutated ras, neutrophil elastase, NKG2D, Nkp30, NY-ESO-1, p53, PAP, prostase, prostate specific antigen (PSA), prostate-carcinoma tumor antigen-1 (PCTA-1), prostate-specific antigen protein, STEAP1, STEAP2, PSMA, RAGE-1, ROR1, RU1, RU2 (AS), surface adhesion molecule, surviving and telomerase, TAG-72, the extra domain A (EDA) and extra domain B (EDB) of fibronectin and the Al domain of tenascin-C (TnC Al), thyroglobulin, tumor stromal antigens, vascular endothelial growth factor receptor-2 (VEGFR2), virus-specific surface antigen such as an HIV-specific antigen (such as HIV gpl20), as well as any derivate or variant of these surface markers.

In some embodiments, the CAR specifically targets antigens selected from the group consisting of BCMA, CD19, CLL1, CS1, STEAP1, STEAP2, CD70, and CD20. In some embodiments, the CAR specifically targets CD19.

In some embodiments, the CAR comprises a costimulatory or spacer domain derived from a molecule selected from the group consisting of 4-1BB/CD137, B7-H3, BAFFR, BLAME (SLAMF8), BTLA, CD 33, CD 45, CD100 (SEMA4D), CD103, CD134, CD137, CD154, CD16, CD160 (BY55), CD18, CD19, CD19a, CD2, CD22, CD247, CD27, CD276 (B7-H3), CD28, CD29, CD3 (alpha; beta; delta; epsilon; gamma; zeta), CD30, CD37, CD4, CD4, CD40, CD49a, CD49D, CD49f, CD5, CD64, CD69, CD7, CD80, CD83 ligand, CD84, CD86, CD8alpha, CD8beta, CD9, CD96 (Tactile), CDl-la, CDl-lb, CDl-lc, CDl-ld, CDS, CEACAM1, CRT AM, DAP-10, DNAM1 (CD226), Fc gamma receptor, GADS, GITR, HVEM (LIGHTR), IA4, ICAM-1, ICAM-1, ICOS, Ig alpha (CD79a), IL2R beta, IL2R gamma, IL7R alpha, integrin, ITGA4, ITGA4, ITGA6, ITGAD, ITGAE, ITGAL, ITGAM, ITGAX, ITGB2, ITGB7, ITGBl, KIRDS2, LAT, LFA-1, LFA-1, LIGHT, LIGHT (tumor necrosis factor superfamily member 14; TNFSF14), LTBR, Ly9 (CD229), lymphocyte function-associated antigen-1 (LFA-1 (CDl la/CD18), MHC class I molecule, NKG2C, NKG2D, NKp30, NKp44, NKp46, NKp80 (KLRF1), OX40, PAG/Cbp, PD-1, PSGL1, SELPLG (CD162), signaling lymphocytic activation molecule, SLAM (SLAMF1; CD150; IPO-3), SLAMF4 (CD244; 2B4), SLAMF6 (NTB-A; Lyl08), SLAMF7, SLP-76, TNF, TNFr, TNFR2, Toll ligand receptor, TRANCE/RANKL, VLA1, and VLA-6, or fragments, truncations, or combinations thereof.

In some embodiments, the CAR comprises a CD19 scFv, a CD28 spacer, CD28 costimulatory domain, and CD3zeta domain.

In some embodiments, the CAR specifically targets two or more antigens.

In some embodiments, the modified pluripotent stem cell comprises an exogenous construct encoding a TCR. In some embodiments, the TCR is an alpha/beta. TCR, gamma/delta TCR, a cancer or cancer associated antigen reactive TCR, TCR that is reactive against murine or other non-human MHC, a class I or class II restricted TCR. In some embodiments, the TCR is derived from CD8, CD4, CD4/8 double positive, immature or developing T cell, Treg, NKT, or NK T cell.

In some embodiments, the TCR is an HPV recognizing TCR, a viral reactive TCR, a CMV TCR, an EBV TCR, an influenza TCR. In some embodiments, the TCR is an HPV-16 E7 TCR. In some embodiments, the TCR is an HPV-16 E6 TCR, MAGEA3/A6 TCR or engineered variant.

In some embodiments, the TCR is linked by an IRES element. In some embodiments, the TCR is linked by a 2A element. In some embodiments, the 2A element is P2A, T2A, E2A, or F2A.

In some embodiments, the TCR is linked by a non-bicistronic approach. In some embodiments, the TCR is integrated at different genomic locations.

In some embodiments, the modified pluripotent stem cell comprises an exogenous construct encoding a suicide gene, wherein the suicide gene allows for the elimination of gene modified cells. In some embodiments, the suicide gene is sr39TK. In some embodiments, the sr39TK is used as a PET reporter gene for non-invasive imaging.

In some embodiments, the suicide gene is a chemically induced caspase, dimerization induced by a small molecule/chemically induced dimerizer (CID), or a selectable surface marker. In some embodiments, the selectable surface marker is CD19, CD20, CD34, EGFR or LNGFR. In some embodiments, the suicide gene is activated in case of an adverse event, self-reactivity of infused cells, eradication of cancer, or other.

In some embodiments, the exogenous construct is a viral construct. In some embodiments, the viral construct is an AAV construct, adenoviral construct, lentiviral construct, or retroviral construct.

In some embodiments, the exogenous construct is integrated into the genome of the stem cell. In some embodiments, the exogenous construct is not integrated into the genome of the stem cell. In some embodiments, the exogenous construct is introduced by a transposase, retrotransposase, episomal plasmid or random integration

In some embodiments, the knockout is created by homologous recombination.

In some embodiments, the modified cell is an induced pluripotent stem cell (iPSC) derived from a T cell or non-T cell. In some embodiments, the T cell derived from alpha beta T cells, gamma delta T cells, NK cells, NKT cells, ILCs, or a Tregs.

In some embodiments, the modified cell is derived from a B cell, peripheral blood mononuclear cell, hematopoietic progenitor, hematopoietic stem cell, mesenchymal stem cell, adipose stem cell, somatic cell type or an embryonic stem cell.

In some embodiments, the modified pluripotent stem cell has no MHC reactivity.

In one aspect, the present disclosure provides a method of generating a modified pluripotent stem cell comprising (a) editing a gene locus to eliminate expression of endogenous TCR or block expression of donor HLA; and (b) introducing an exogenous construct encoding a CAR, TCR, or HLA gene.

In some embodiments, the method further comprises a step of first isolating a hematopoietic stem cell, an embryonic stem, or an induced pluripotent stem cell.

In some embodiments, the method comprises editing the endogenous TCRα constant (TRAC) gene, beta constant (TRBC) gene or beta 2 microglobulin (b2m) gene.

In some embodiments, the edited gene is created by knockout.

In some embodiments, the gene is edited using CRISPR/Cas9, a zinc finger nuclease (ZFN), a TALEN, a MegaTAL, a meganuclease, Cpf1, homologous recombination, or a single stranded oligodeoxynucleotide (ssODN). In some embodiments, the gene is edited using a zinc finger nuclease (ZFN).

In some embodiments, the exogenous construct encodes a single chain HLA trimer. In some embodiments, the single chain HLA trimer comprises an HLA class I HLA-E. In some embodiments, the single chain HLA trimer comprises an HLA linked to beta-2-microglobulin linked to a stabilizing peptide. In some embodiments, the HLA trimer is HLA-E, HLA-G, or a combination of HLA-E and HLA-G.

In some embodiments, the exogenous construct encodes a chimeric antigen receptor (CAR). In some embodiments, the CAR specifically targets antigens selected from the group consisting of BCMA, CD19, CLL1, CS1, STEAP1, STEAP2, CD70, or CD20.

In some embodiments, the CAR specifically targets CD19. In some embodiments, the CAR comprises a CD19 scFv, a CD28 spacer, CD28 costimulatory domain, and CD3zeta.

In some embodiments, the CAR specifically targets two or more antigens.

In some embodiments, the exogenous construct encodes a TCR.

In some embodiments, the TCR is derived from an alpha/beta TCR, gamma/delta TCR, a cancer or cancer associated antigen reactive TCR, TCR that is reactive against murine or other non-human MHC, a class I or class II restricted TCR. In some embodiments, the TCR is a hybrid or engineered TCR.

In some embodiments, the TCR is an HPV recognizing TCR, a viral reactive TCR, an EBV TCR, an influenza TCR.

In some embodiments, the TCR is an HPV-16 E7 TCR, an HPV-16 E6 or a MAGEA3/A6 TCR or engineered variant.

In some embodiments, the TCR is linked by an IRES element.

In some embodiments, the TCR is linked by a 2A element.

In some embodiments, the 2A element is P2A, T2A, E2A, or F2A.

In some embodiments, the TCR is linked by a non-bicistronic approach.

In some embodiments, the each chain of the TCR is integrated at different genomic locations.

In some embodiments, the exogenous construct encodes a suicide gene, wherein the suicide gene allows for the elimination of gene modified cells. In some embodiments, the suicide gene is sr39TK. In some embodiments, the suicide gene is a chemically induced caspase, dimerization induced by a small molecule/chemically induced dimerizer (CID), or a selectable surface marker.

In some embodiments, the selectable surface marker is CD19, CD20, CD34, EGFR or LNGFR.

In some embodiments, the exogenous construct is a viral construct.

In some embodiments, the viral construct is an AAV construct, adenoviral construct, lentiviral construct, or retroviral construct.

In some embodiments, the exogenous construct is integrated into the genome of the stem cell. In some embodiments, the exogenous construct is not integrated into the genome of the stem cell. In some embodiments, the exogenous construct is not integrated into the genome of the stem cell. In some embodiments, the exogenous construct is introduced by a transposase, retrotransposase, episomal plasmid or random integration

In some embodiments, the knockout is created by homologous recombination.

In some embodiments, the modified cell is an induced pluripotent stem cell (iPSC) derived from a T cell or non-T cell. In some embodiments, the T cell derived from alpha beta T cells, gamma delta T cells, NK cells, NKT cells, ILCs, or a Tregs.

In one aspect, the present disclosure provides a method of generating a T cell lineage of interest, comprising steps of (a) providing a modified pluripotent stem cell described herein, and (b) inducing T cell or T cell-like differentiation.

In some embodiments, the T cell differentiation is induced using an artificial thymic organoid (ATO) system, notch agonist, OP9-DLL1, OP9-DLL4, fetal thymic organoid culture (FTOC), chemical induction, bone marrow/liver/thymus or other humanized mouse, embryoid body (EB).

In some embodiments, the T cell lineage is selected by detecting expression of one or more biomarkers.

In some embodiments, the T cell lineage is selected by detecting expression of one or more biomarkers, optionally, wherein the T cell lineage of interest is a CD8 single positive T cell, a CD4 single positive T cell, a CD4 CD8 double positive T cell, a double negative T cell, a CD3 positive cell, an NK cell, a proT cell, a pre-proT cell, a mesodermal progenitor, a B cell, a common lymphoid progenitor, a hematopoietic progenitor, a hematopoietic stem cell.

In some embodiments, the T cell lineage of interest is a CD8 single positive T cell, a CD4 single positive T cell, a CD4 CD8 double positive T cell, a double negative T cell, a CD3 positive cell, an NK cell, a proT cell, a pre-proT cell, a mesodermal progenitor, a B cell, a common lymphoid progenitor, a hematopoietic progenitor, a hematopoietic stem cell.

In one aspect, the present disclosure provides a method of generating a T cell lineage of interest, comprising (a) providing a modified pluripotent stem cell described herein, (b) editing a gene encoding a cell fate regulator to promote, impair or eliminate the generation of a specific cell lineage, and (c) inducing T cell differentiation.

In some embodiments, the cell fate regulator is a transcription factor, T-BET, STAT1, STAT4, STAT, RUNX3, GATA3, Stat5, Stat6, DEC2, MAF, THPOK, GATA3, Smads, Stat6, PU.1, RORgt, RORa, Stat3, AHR, Bcl-6, MAF, FoxP3, Smad3, Stat5, FOXO1, FOXO3, GRAIL, or PLZF.

In some embodiments, the specific lineage is Th1, Th2, Th9, Th17, Th22, Tfh, Treg, ILC, NK, or NKT.

In one aspect, the present disclosure provides a method of generating a T cell lineage of interest, comprising (a) providing a modified pluripotent stem cell described herein, (b) editing a cell fate regulator to impair or eliminate the generation of undesired cell lineage, and (c) inducing T cell differentiation.

In some embodiments, the T cell differentiation is induced using an artificial thymic organoid (ATO) system, notch agonist, OP9-DLL1, OP9-DLL4, fetal thymic organoid culture (FTOC), chemical induction, bone marrow/liver/thymus or other humanized mouse, embryoid body (EB).

In some embodiments, the T cell lineage is selected by detecting expression of one or more biomarkers.

In some embodiments, the T cell lineage of interest is a CD8 single positive T cell, a CD4 single positive T cell, a CD4 CD8 double positive T cell, a double negative T cell, a CD3 positive cell, an NK cell, an NKT cell a proT cell, a pre-proT cell, a mesodermal progenitor, a B cell, a common lymphoid progenitor, a hematopoietic progenitor, a hematopoietic stem cell.

In some embodiments, the cell fate regulator is a transcription factor.

In some embodiments, the undesired lineage is Th1, Th2, Th9, Th17, Th22, Tfh, Treg, ILC, NK or NKT.

In some embodiments, the cell fate regulator is T-BET, STAT1 STAT4, STAT, or RUNX3.

In some embodiments, the cell fate regulator is GATA3, Stat5, Stat6, DEC2, MAF, or THPOK.

In some embodiments, the cell fate regulator is GATA3, Smads, Stat6, or PU.1.

In some embodiments, the cell fate regulator is RORgt, RORa, or Stat3.

In some embodiments, the cell fate regulator is AHR.

In some embodiments, the cell fate regulator is Bcl-6, or MAF.

In some embodiments, the cell fate regulator is FoxP3, Smad3, Stat5, FOXO1, FOXO3, or GRAIL.

In some embodiments, the cell fate regulator is PLZF.

In one aspect, the present disclosure provides a method of generating a T cell lineage of interest, comprising steps of (a) providing a modified pluripotent stem cell described herein, (b) editing a cell fate regulator to promote the generation of a desired cell lineage, and, (c) inducing T cell differentiation.

In some embodiments, T cell differentiation is induced using an artificial thymic organoid (ATO) system, notch agonist, OP9-DLL1, OP9-DLL4, fetal thymic organoid culture (FTOC), chemical induction, bone marrow, liver, thymus or other humanized mouse, embryoid body (EB).

In some embodiments, the T cell lineage is selected by detecting expression of one or more biomarkers.

In some embodiments, the T cell lineage of interest is a CD8 single positive T cell, a CD4 single positive T cell, a CD4 CD8 double positive T cell, a double negative T cell, a CD3 positive cell, an NK cell, an NKT cell, a proT cell, a pre-proT cell, a mesodermal progenitor, a B cell, a common lymphoid progenitor, a hematopoietic progenitor, a hematopoietic stem cell.

In some embodiments, the cell fate regulator is a transcription factor.

In some embodiments, the desired lineage is Th1, Th2, Th9, Th17, Th22, Tfh, Treg, ILC, NK, or NKT.

In some embodiments, the cell fate regulator is T-BET, STAT1, STAT4, STAT, or RUNX3.

In some embodiments, the cell fate regulator is GATA3, Stat5, Stat6, DEC2, MAF, or THPOK.

In some embodiments, the cell fate regulator is GATA3, Smads, Stat6, or PU.1.

In some embodiments, wherein the cell fate regulator is RORgt, RORa, or Stat3.

In some embodiments, the cell fate regulator is AHR.

In some embodiments, the cell fate regulator is Bcl-6, or MAF.

In some embodiments, the cell fate regulator is FoxP3, Smad3, Stat5, FOXO1, FOXO3, or GRAIL.

In some embodiments, the cell fate regulator is PLZF.

In some embodiments, engineered stem cells further express chimeric antigen receptors (CARs) or T cell receptors (TCRs) which specifically target and kill cancer cells.

A CAR may comprise, for example, (i) an antigen-specific component (“antigen binding molecule”), (ii) one or more costimulatory domains (which includes a hinge domain), and (iii) one or more activating domains. Each domain may be heterogeneous, that is, comprised of sequences derived from different protein chains. CAR-expressing immune cells (such as T cells) may be used in various therapies, including cancer therapies.

TCRs are proteins that allow T cells to identify cancer targets presented on the surface of cancer cells or inside cancer cells. Endogenous TCRs that are specific to a cancer may be isolated and then engineered into a large number of T cells that recognize and attack various types of solid and hematologic cancers.

In some embodiments, a CAR may contain a transmembrane domain selected from the group transmembrane domain of 4-1BB/CD137, an alpha chain of a T cell receptor, a beta chain of a T cell receptor, a gamma chain of a T cell receptor, a delta chain of a T cell receptor, CD3 epsilon, CD3 delta, CD3 gamma, CD3 zeta, CD4, CD5, CD8 alpha, CD9, CD16, CD19, CD22, CD33, CD34, CD37, CD45, CD64, CD80, CD86, CD134, CD137, CD154, or a zeta chain of a T cell receptor, or any combination thereof.

In some embodiments, the intracellular domain comprises a signaling region of 4-1BB/CD137, activating NK cell receptors, B7-H3, BAFFR, BLAME (SLAMF8), BTLA, CD100 (SEMA4D), CD103, CD160 (BY55), CD18, CD19, CD19a, CD2, CD247, CD27, CD276 (B7-H3), CD29, CD3 delta, CD3 epsilon, CD3 gamma, CD30, CD4, CD40, CD49a, CD49D, CD49f, CD69, CD7, CD84, CD8alpha, CD8beta, CD96 (Tactile), CDl la, CDl lb, CDl lc, CDl ld, CDS, CEACAM1, CRT AM, cytokine receptors, DAP-10, DNAM1 (CD226), Fc gamma receptor, GADS, GITR, HVEM (LIGHTR), IA4, ICAM-1, Ig alpha (CD79a), IL2R beta, IL2R gamma, IL7R alpha, Immunoglobulin-like proteins,

In some embodiments, the cancer is acute lymphoblastic leukemia (ALL) (including non T cell ALL), acute myeloid leukemia, B cell prolymphocytic leukemia, B-cell acute lymphoid leukemia (“BALL”), blastic plasmacytoid dendritic cell neoplasm, Burkitt's lymphoma, chronic lymphocytic leukemia (CLL), chronic myelogenous leukemia (CML), chronic myeloid leukemia, chronic or acute leukemia, diffuse large B cell lymphoma (DLBCL), follicular lymphoma (FL), hairy cell leukemia, Hodgkin's Disease, malignant lymphoproliferative conditions, MALT lymphoma, mantle cell lymphoma, Marginal zone lymphoma, monoclonal gammapathy of undetermined significance (MGUS), multiple myeloma, myelodysplasia and myelodysplastic syndrome, non-Hodgkin's lymphoma (NHL), plasma cell proliferative disorder (including asymptomatic myeloma (smoldering multiple myeloma or indolent myeloma), plasmablastic lymphoma, plasmacytoid dendritic cell neoplasm, plasmacytomas (including plasma cell dyscrasia; solitary myeloma; solitary plasmacytoma; extramedullary plasmacytoma; and multiple plasmacytoma), POEMS syndrome (also known as Crow-Fukase syndrome; Takatsuki disease; and PEP syndrome), primary mediastinal large B cell lymphoma (PMBC), small cell- or a large cell-follicular lymphoma, splenic marginal zone lymphoma (SMZL), systemic amyloid light chain amyloidosis, T-cell acute lymphoid leukemia (“TALL”), T-cell lymphoma, transformed follicular lymphoma, or Waldenstrom macroglobulinemia, or a combination thereof.

In one aspect, the present invention provides modified pluripotent stem cells with enriched pairing between a pre-TCRα (pTa) protein and a TCRβ protein as compared to an unmodified control cell.

In some embodiments, the modified pluripotent stem cell comprises an exogenous construct encoding the pre-TCRα (pTa) protein, optionally, wherein the exogenous construct is a viral construct, an AAV construct, lentiviral construct, or retroviral construct.

In some embodiments, the modified pluripotent stem cell comprises an exogenous construct encoding the pre-TCRα (pTa) protein.

In some embodiments, the modified pluripotent stem cell comprises an exogenous construct, wherein the exogenous construct is a viral construct. In some embodiments, the modified pluripotent stem cell comprises an exogenous viral construct, wherein the viral construct is an AAV construct, lentiviral construct, or retroviral construct.

In some embodiments, the modified pluripotent stem cell comprises an exogenous construct that is integrated into the genome of the stem cell.

In some embodiments, the modified pluripotent stem cell comprises a deficient TCRα gene. In some embodiments, the deficient TCRα gene is created by knockout. In certain embodiments, TCRα gene knockout is created by an engineered nuclease. In some embodiments, the engineered nuclease is specific to the TCRα gene and is selected from TALEN, megaTAL, CRISPR, ZFN.

In other embodiments, the modified pluripotent stem cell comprises a TCRα gene knockout, wherein the knockout is created by homologous recombination. In certain embodiments, the deficient TCRα gene is created by antisense RNA.

In some embodiments, the modified pluripotent stem cell is substantially free of TCRα and TCRβ pairing.

In some embodiments, the modified pluripotent stem cell further comprises a chimeric antigen receptor (CAR), an exogenous TCR, and/or an antigen receptor.

In some embodiments, a hematopoietic stem cell, an embryonic stem, or an induced pluripotent stem cell is used to generate the modified pluripotent stem cell. In some embodiments, the modified pluripotent stem cell has no MHC reactivity.

In one aspect, the present invention provides a method of generating a modified pluripotent stem cell comprising a step of introducing an exogenous pre-TCRα (pTa) protein and/or creating a deficient TCRα gene.

In some embodiments, the exogenous pre-TCRα (pTa) protein is introduced by electroporation of a DNA or RNA construct encoding the pre-TCRα (pTa) protein.

In some embodiments, the deficient TCRα gene is created by a knockout or antisense technique. In certain embodiments, the method further comprises a step of introducing a construct encoding a CAR protein of interest.

In some embodiments, the method further comprises a step of first isolating a hematopoietic stem cell, an embryonic stem, or an induced pluripotent stem cell from a patient or healthy donor.

In one aspect, the present invention provides a method of generating a T cell lineage of interest; comprising steps of providing a modified pluripotent stem cell and inducing T cell differentiation in an artificial thymic organoid.

In one aspect, the present invention provides a method of generating a T cell lineage of interest, comprising providing a modified pluripotent stem cell described herein, and inducing T cell differentiation in the presence or absence of peptide:MHC, optionally, wherein the T cell lineage of interest is cytotoxic CD8+ T cells, helper CD4+ T cells, helper CD4+ T cells that are Th1/Th2/Th17 cells, regulatory T cells, intra epithelial lymphocyte (IEL), or mature alpha-beta or gamma-delta T cell.

In one aspect, the present invention provides a method of generating a T cell lineage of interest; comprising steps of providing a modified pluripotent stem cell and inducing T cell differentiation in the presence of peptide:MHC. In one aspect, the present invention provides a method of generating a T cell lineage of interest; comprising steps of providing a modified pluripotent stem cell and inducing T cell differentiation in the absence of peptide:MHC.

In some embodiments, the method further comprises selecting a T cell lineage, wherein the T cell lineage is selected by detecting expression of one or more biomarkers. In some embodiments, the T cell lineage of interest is cytotoxic CD8+ T cells. In some other embodiments, the T cell lineage of interest is helper CD4+ T cells. In certain embodiments, the helper CD4+ T cells are Th1/Th2/Th17 cells.

in some embodiments, the method comprises selecting a T cell lineage, wherein the T cell lineage of interest is regulatory T cell.

In some embodiments, the method comprises selecting a T cell lineage, wherein the T cell lineage of interest is intra epithelial lymphocyte (IEL).

In some embodiments, the method comprises selecting a T cell lineage, wherein the T cell lineage of interest is mature alpha-beta or gamma-delta T cell.

A CAR may comprise, for example, (i) an antigen-specific component (“antigen binding molecule”), (ii) one or more costimulatory domains (which includes a hinge domain), and (iii) one or more activating domains. Each domain may be heterogeneous, that is, comprised of sequences derived from different protein chains. CAR-expressing immune cells (such as T cells) may be used in various therapies, including cancer therapies.

CARs comprising a costimulatory domain, which includes a truncated hinge domain (“THD”), provides unexpectedly superior properties when compared to a CAR comprising a costimulatory domain, which includes a complete hinge domain (“CHD”). Polynucleotides encoding such CARs may be transduced into engineered stem cells of the present invention comprising pTA, and TCRα, or endogenous stem cells lacking TCRα. When the transduced T cells are transplanted to a patient, the CARs direct the T cells to recognize and bind an epitope present on the surface of cancer cells, thus, allowing binding of cancer cells rather than non-cancerous cells. This binding leads to activation of cytolytic mechanisms in the T cell that specifically kill the bound cancer cells. The medical complication graft-versus-host disease (GvHD) is commonly associated with stem cell transplant, which may be treated with immunosuppressive therapy. The present invention potentially eliminates the possibility of developing GvHD by generating modified T cells that retain antigen specificity, but without reactivity to major histocompatibility complex (MHC) molecules. Thus, the present invention satisfies an unmet need that exists for novel and improved therapies for treating cancer.

TCRs are proteins that allow T cells to identify cancer targets presented on the surface of cancer cells or inside cancer cells. Endogenous TCRs that are specific to a cancer may be isolated and then engineered into a large number of T cells that recognize and attack various types of solid and hematologic cancers.

In some embodiments, CAR may contain a transmembrane domain selected from the group transmembrane domain of 4-1BB/CD137, an alpha chain of a T cell receptor, a beta chain of a T cell receptor, a gamma chain of a T cell receptor, a delta chain of a T cell receptor, CD3 epsilon, CD3 delta, CD3 gamma, CD3 zeta, CD4, CD5, CD8 alpha, CD9, CD16, CD19, CD22, CD33, CD34, CD37, CD45, CD64, CD80, CD86, CD134, CD137, CD154, or a zeta chain of a T cell receptor, or any combination thereof.

In some embodiments, the intracellular domain comprises a signaling region of 4-1BB/CD137, activating NK cell receptors, B7-H3, BAFFR, BLAME (SLAMF8), BTLA, CD100 (SEMA4D), CD103, CD160 (BY55), CD18, CD19, CD19a, CD2, CD247, CD27, CD276 (B7-H3), CD29, CD3 delta, CD3 epsilon, CD3 gamma, CD30, CD4, CD40, CD49a, CD49D, CD49f, CD69, CD7, CD84, CD8alpha, CD8beta, CD96 (Tactile), CDl la, CDl lb, CDl lc, CDl ld, CDS, CEACAM1, CRT AM, cytokine receptors, DAP-10, DNAM1 (CD226), Fc gamma receptor, GADS, GITR, HVEM (LIGHTR), IA4, ICAM-1, ICAM-1, Ig alpha (CD79a), IL2R beta, IL2R gamma, IL7R alpha, Immunoglobulin-like proteins,

In some embodiments, the cancer is acute lymphoblastic leukemia (ALL) (including non T cell ALL), acute myeloid leukemia, B cell prolymphocytic leukemia, B-cell acute lymphoid leukemia (“BALL”), blastic plasmacytoid dendritic cell neoplasm, Burkitt's lymphoma, chronic lymphocytic leukemia (CLL), chronic myelogenous leukemia (CML), chronic myeloid leukemia, chronic or acute leukemia, diffuse large B cell lymphoma (DLBCL), follicular lymphoma (FL), hairy cell leukemia, Hodgkin's Disease, malignant lymphoproliferative conditions, MALT lymphoma, mantle cell lymphoma, Marginal zone lymphoma, monoclonal gammapathy of undetermined significance (MGUS), multiple myeloma, myelodysplasia and myelodysplastic syndrome, non-Hodgkin's lymphoma (NHL), plasma cell proliferative disorder (including asymptomatic myeloma (smoldering multiple myeloma or indolent myeloma), plasmablastic lymphoma, plasmacytoid dendritic cell neoplasm, plasmacytomas (including plasma cell dyscrasia; solitary myeloma; solitary plasmacytoma; extramedullary plasmacytoma; and multiple plasmacytoma), POEMS syndrome (also known as Crow-Fukase syndrome; Takatsuki disease; and PEP syndrome), primary mediastinal large B cell lymphoma (PMBC), small cell- or a large cell-follicular lymphoma, splenic marginal zone lymphoma (SMZL), systemic amyloid light chain amyloidosis, T-cell acute lymphoid leukemia (“TALL”), T-cell lymphoma, transformed follicular lymphoma, or Waldenstrom macroglobulinemia, or a combination thereof.

As described herein, the modified pluripotent stem cells may be used in an allogenic setting or in Engineered Autologous Cell Therapy, abbreviated as “eACT™,” also known as adoptive cell transfer. eACT™, is a process by which a patient's own T cells are collected and subsequently genetically engineered to recognize and target one or more antigens expressed on the cell surface of one or more specific cancer cells. T cells may be engineered to express, for example, a CAR or TCR. CAR. positive (CAR+) T cells are engineered to express a CAR. CARs may comprise, e.g., an extracellular single chain variable fragment (scFv) with specificity for a particular tumor antigen, which is directly or indirectly linked to an intracellular signaling part comprising at least one costimulatory domain, which is directly or indirectly linked to at least one activating domain; the components may be arranged in any order. The costimulatory domain may be derived from a costimulatory protein known in the art, and the activating domain may be derived from, e.g., any form of CD3-zeta. In some embodiments, the CAR is designed to have two, three, four, or more costimulatory domains. In some embodiments, a CAR is engineered such that the costimulatory domain is expressed as a separate polypeptide chain. Examples of CAR T cell therapies and constructs are described in U.S. Patent Publication Nos. 2013/0287748, 2014/0227237, 2014/0099309, and 2014/0050708; International Patent Publications Nos. WO2012033885, WO2012079000, WO2014127261, WO2014186469, WO2015080981, WO2015142675, WO2016044745, and WO2016090369; and Sadelain et al, Cancer Discovery, 3: 388-398 (2013), each of which is incorporated by reference in its entirety.

Any aspect or embodiment described herein may be combined with any other aspect or embodiment as disclosed herein. While the present invention has been described in conjunction with the detailed description thereof, the foregoing description is intended to illustrate and not limit the scope of the present invention, which is defined by the scope of the appended claims. Other aspects, advantages, and modifications are within the scope of the following claims.

The patent and scientific literature referred to herein establishes the knowledge that is available to those with skill in the art. All United States patents and published or unpublished United States patent applications cited herein are incorporated by reference. All published foreign patents and patent applications cited herein are hereby incorporated by reference. All other published references, dictionaries, documents, manuscripts, genomic database sequences, and scientific literature cited herein are hereby incorporated by reference.

Other features and advantages of the invention will be apparent from the Drawings and the following Detailed Description, including the Examples, and the claims.

### BRIEF DESCRIPTION OF THE DRAWINGS

The above and further features will be more clearly appreciated from the following detailed description when taken in conjunction with the accompanying drawings. The drawings however are for illustration purposes only, not for limitation.

FIG. 1 shows a schematic demonstrating the production of engineered T cells from modified pluripotent stem cells and an exemplary modification strategy.

FIG. 2 shows an exemplary modification strategy.

FIG. 3 shows a schematic representation of the elimination of cell by-products targeted gene editing. On the left, a normal differentiation tree of a normal stem cell to a T cell. On the right, the edited stem cells do not produce undesired cell by-products, only the final T cell.

FIG. 4 shows an experimental schematic of the ATO system. Pluripotent stem cells are induced to mesodermal progenitors. Mesoderm progenitors are sorted, and complexed with MS5 stromal cells engineered to express DLL1. Aggregate cell complexes are dropped onto an air-liquid-interface membrane, and allowed to develop to T cells over 8-12 weeks.

FIG. 5 shows kinetics of T Cell Development from iPSC in ATO. iPSC gain surface markers CD45, CD5, and CD7 characteristic of T lineage committed cells. Cells are initially (week 2) CD4ISP or CD4/8DP. At week 3, all cells are CD4/8DP. By week 5, the majority of cells are expressing an alpha-beta T-cell receptor, and are CD8SP.

FIG. 6 shows the expansion of sorted cells. CD4 single positive (CD4SP), CD 4/8 double positive (DP), and CD8 single positive (CD8SP) cells were sorted at the end of ATO development. Sorted populations were expanded in Optimizer medium with IL2, and CD3/28 beads for 2 weeks. At the end of expansion, cells were enumerated to calculate fold-expansion. Two replicate experiments are shown (ATO20 and ATO21).

FIG. 7 shows activation markers. Healthy donor control cells were cultured overnight in untreated plates, or plates coated with OKT3 (CD3 stimulating antibody). Expression of surface markers in either CD4 or CD8 populations was investigated by flow cytometry. Upregulation of CD69 and 4-1BB was observed on cells cultured with OKT3.

FIG. 8 shows activation markers in iPSC derived T cells from ATO. As in healthy donor cells (FIG. 7), T cells derived from iPSC in ATO show upregulation of surface markers CD69 and 4-1BB after overnight co-culture on OKT3 coated plates.

FIG. 9 shows activation markers summary and proliferation. Left graphs summarize data from FIGS. 7 and 8. Right graphs show the dilution of CellTrace Violet in stimulated cells, indicating that proliferation was induced when cells were cultured on OKT3. Proliferation upon stimulus is a hallmark of T cell function.

FIG. 10 shows secretion of cytokines. Immune cytokines IFNg, IL2, TNFa, IL-8 and IL-10 were secreted by healthy donor controls and T-cells generated from iPSCs in ATO upon stimulation with OKT3. Secretion of cytokines upon stimulus is a hallmark of T cell function.

FIG. 11 shows CD19 CAR expressing T-cells derived from iPSCs are functional against targets. T-cells manufactured to express CD19 CAR in Kite's manufacturing process (AxiCel) or T-cells developed from CD19-CAR transduced iPSCs were co-cultured with CD19+ leukemic target cells (Raji) overnight. Cells formed clusters (left), and upregulated the surface marker 4-1BB (middle, right) when effectors and targets were co-cultured. T-cells from CD19 CAR transduced iPSCs demonstrate functional recognition of target cancer lines.

FIG. 12 shows iPSC are able generate mesodermal progenitors after CAR transduction or Gene Editing. The parental (202i) iPSC line was transduced with CD19 CAR (EFLbright) and sorted to clones (Clone 2, Clone 5, Clone 8, Clone 11), or gene edited to eliminate expression of beta2microglobulin and sorted to clones (b2m R2, b2m R6, b2m R9, b2m Y3). All transduced or gene edited lines or clones were able to form mesodermal progenitors at an efficiency comparable to the parental line.

FIG. 13 shows the developmental phases of T cell differentiation.

FIG. 14 shows the early stages of double negative (DN) and double positive (DP) thymocyte development.

FIG. 15 shows a schematic representing the molecular organization of surface expressed TCRs.

FIG. 16A-16C show flow cytometry plots illustrating T cell differentiation at week 5 of non-modified iPSC (FIG. 16A), CAR-KI-TRAC iPSC (FIG. 16B) and CD45+CD56−CD3+CAR+E7TCRab+ T cells from modified iPSC (FIG. 16C).

### DEFINITIONS

In order for the present invention to be more readily understood, certain terms are first defined below. Additional definitions for the following terms and other terms are set forth throughout the Specification.

As used in this Specification and the appended claims, the singular forms “a,” “an” and “the” include plural referents unless the context clearly dictates otherwise.

Unless specifically stated or obvious from context, as used herein, the term “or” is understood to be inclusive and covers both “or” and “and.”

The term “and/or” where used herein is to be taken as specific disclosure of each of the two specified features or components with or without the other. Thus, the term “and/or” as used in a phrase such as “A and/or B” herein is intended to include A and B; A or B; A (alone), and B (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

The terms “e.g.,” and “i.e.” as used herein, are used merely by way of example, without limitation intended, and should not be construed as referring only those items explicitly enumerated in the specification.

The terms “or more”, “at least”, “more than”, and the like, e.g., “at least one” are understood to include but not be limited to at least 1, 2, 3, 4, 5, 6, 7, 8, 9, 10, 11, 12, 13, 14, 15, 16, 17, 18, 19 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149 or 150, 200, 300, 400, 500, 600, 700, 800, 900, 1000, 2000, 3000, 4000, 5000 or more than the stated value. Also included is any greater number or fraction in between.

Conversely, the term “no more than” includes each value less than the stated value. For example, “no more than 100 nucleotides” includes 100, 99, 98, 97, 96, 95, 94, 93, 92, 91, 90, 89, 88, 87, 86, 85, 84, 83, 82, 81, 80, 79, 78, 77, 76, 75, 74, 73, 72, 71, 70, 69, 68, 67, 66, 65, 64, 63, 62, 61, 60, 59, 58, 57, 56, 55, 54, 53, 52, 51, 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2, 1, and 0 nucleotides. Also included is any lesser number or fraction in between.

The terms “plurality”, “at least two”, “two or more”, “at least second”, and the like, are understood to include but not limited to at least 2, 3, 4, 5, 6, 7, 8, 9, 10, 11, 12, 13, 14, 15, 16, 17, 18, 19 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149 or 150, 200, 300, 400, 500, 600, 700, 800, 900, 1000, 2000, 3000, 4000, 5000 or more. Also included is any greater number or fraction in between.

Throughout the specification the word “comprising,” or variations such as “comprises” or “comprising,” will be understood to imply the inclusion of a stated element, integer or step, or group of elements, integers or steps, but not the exclusion of any other element, integer or step, or group of elements, integers or steps. It is understood that wherever aspects are described herein with the language “comprising,” otherwise analogous aspects described in terms of “consisting of” and/or “consisting essentially of” are also provided.

Unless specifically stated or evident from context, as used herein, the term “about” refers to a value or composition that is within an acceptable error range for the particular value or composition as determined by one of ordinary skill in the art, which will depend in part on how the value or composition is measured or determined, i.e., the limitations of the measurement system. For example, “about” or “comprising essentially of” may mean within one or more than one standard deviation per the practice in the art. “About” or “comprising essentially of” may mean a range of up to 10% (i.e., ±10%). Thus, “about” may be understood to be within 10%, 9%, 8%, 7%, 6%, 5%, 4%, 3%, 2%, 1%, 0.5%, 0.1%, 0.05%, 0.01%, or 0.001% greater or less than the stated value. For example, about 5 mg may include any amount between 4.5 mg and 5.5 mg. Furthermore, particularly with respect to biological systems or processes, the terms may mean up to an order of magnitude or up to 5-fold of a value. When particular values or compositions are provided in the instant disclosure, unless otherwise stated, the meaning of “about” or “comprising essentially of” should be assumed to be within an acceptable error range for that particular value or composition.

As described herein, any concentration range, percentage range, ratio range or integer range is to be understood to be inclusive of the value of any integer within the recited range and, when appropriate, fractions thereof (such as one-tenth and one-hundredth of an integer), unless otherwise indicated.

Units, prefixes, and symbols used herein are provided using their Système International de Unites (SI) accepted form. Numeric ranges are inclusive of the numbers defining the range.

Unless defined otherwise, all technical and scientific terms used herein have the same meaning as commonly understood by one of ordinary skill in the art to which this disclosure is related. For example, Juo, “The Concise Dictionary of Biomedicine and Molecular Biology”, 2^{nd} ed., (2001), CRC Press; “The Dictionary of Cell & Molecular Biology”, 5^{th} ed., (2013), Academic Press; and “The Oxford Dictionary Of Biochemistry And Molecular Biology”, Cammack et al. eds., 2^{nd} ed., (2006), Oxford University Press, provide those of skill in the art with a general dictionary for many of the terms used in this disclosure.

“Administering” refers to the physical introduction of an agent to a subject, using any of the various methods and delivery systems known to those skilled in the art. Exemplary routes of administration for the formulations disclosed herein include intravenous, intramuscular, subcutaneous, intraperitoneal, spinal or other parenteral routes of administration, for example by injection or infusion. The phrase “parenteral administration” as used herein means modes of administration other than enteral and topical administration, usually by injection, and includes, without limitation, intravenous, intramuscular, intra-arterial, intrathecal, intralymphatic, intralesional, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion, as well as in vivo electroporation. In some embodiments, the formulation is administered via a non-parenteral route, e.g., orally. Other non-parenteral routes include a topical, epidermal, or mucosal route of administration, for example, intranasally, vaginally, rectally, sublingually or topically. Administering may also be performed, for example, once, a plurality of times, and/or over one or more extended periods.

The term “antibody” (Ab) includes, without limitation, a glycoprotein immunoglobulin, which binds specifically to an antigen. In general, and antibody may comprise at least two heavy (H) chains and two light (L) chains interconnected by disulfide bonds, or an antigen-binding molecule thereof. Each H chain comprises a heavy chain variable region (abbreviated herein as VH) and a heavy chain constant region. The heavy chain constant region comprises three constant domains, CH1, CH2, and CH3. Each light chain comprises a light chain variable region (abbreviated herein as VL) and a light chain constant region. The light chain constant region is comprises one constant domain, CL. The VH and VL regions may be further subdivided into regions of hypervariability, termed complementarity determining regions (CDRs), interspersed with regions that are more conserved, termed framework regions (FR). Each VH and VL comprises three CDRs and four FRs, arranged from amino-terminus to carboxy-terminus in the following order: FR1, CDR1, FR2, CDR2, FR3, CDR3, and FR4. The variable regions of the heavy and light chains contain a binding domain that interacts with an antigen. The constant regions of the Abs may mediate the binding of the immunoglobulin to host tissues or factors, including various cells of the immune system (e.g., effector cells) and the first component (C1q) of the classical complement system.

Antibodies may include, for example, monoclonal antibodies, recombinantly produced antibodies, monospecific antibodies, multispecific antibodies (including bispecific antibodies), human antibodies, engineered antibodies, humanized antibodies, chimeric antibodies, immunoglobulins, synthetic antibodies, tetrameric antibodies comprising two heavy chain and two light chain molecules, an antibody light chain monomer, an antibody heavy chain monomer, an antibody light chain dimer, an antibody heavy chain dimer, an antibody light chain-antibody heavy chain pair, intrabodies, antibody fusions (sometimes referred to herein as “antibody conjugates”), heteroconjugate antibodies, single domain antibodies, monovalent antibodies, single chain antibodies or single-chain Fvs (scFv), camelized antibodies, affybodies, Fab fragments, F(ab′)₂ fragments, disulfide-linked Fvs (sdFv), anti-idiotypic (anti-Id) antibodies (including, e.g., anti-anti-Id antibodies), minibodies, domain antibodies, synthetic antibodies (sometimes referred to herein as “antibody mimetics”), and antigen-binding fragments of any of the above. In certain embodiments, antibodies described herein refer to polyclonal antibody populations.

An immunoglobulin may derive from any of the commonly known isotypes, including but not limited to IgA, secretory IgA, IgG and IgM. IgG subclasses are also well known to those in the art and include but are not limited to human IgG1, IgG2, IgG3 and IgG4. “Isotype” refers to the Ab class or subclass (e.g., IgM or IgG1) that is encoded by the heavy chain constant region genes. The term “antibody” includes, by way of example, both naturally occurring and non-naturally occurring Abs; monoclonal and polyclonal Abs; chimeric and humanized Abs; human or nonhuman Abs; wholly synthetic Abs; and single chain Abs. A nonhuman Ab may be humanized by recombinant methods to reduce its immunogenicity in man. Where not expressly stated, and unless the context indicates otherwise, the term “antibody” also includes an antigen-binding fragment or an antigen-binding portion of any of the aforementioned immunoglobulins, and includes a monovalent and a divalent fragment or portion, and a single chain Ab.

An “antigen binding molecule,” “antigen binding portion,” or “antibody fragment” refers to any molecule that comprises the antigen binding parts (e.g., CDRs) of the antibody from which the molecule is derived. An antigen binding molecule may include the antigenic complementarity determining regions (CDRs). Examples of antibody fragments include, but are not limited to, Fab, Fab′, F(ab′)2, and Fv fragments, dAb, linear antibodies, scFv antibodies, and multispecific antibodies formed from antigen binding molecules. Peptibodies (i.e., Fc fusion molecules comprising peptide binding domains) are another example of suitable antigen binding molecules. In some embodiments, the antigen binding molecule binds to an antigen on a tumor cell. In some embodiments, the antigen binding molecule binds to an antigen on a cell involved in a hyperproliferative disease or to a viral or bacterial antigen. In certain embodiments, the antigen binding molecule binds to BCMA, CLL-1, or FLT3. In further embodiments, the antigen binding molecule is an antibody fragment that specifically binds to the antigen, including one or more of the complementarity determining regions (CDRs) thereof. In further embodiments, the antigen binding molecule is a single chain variable fragment (scFv). In some embodiments, the antigen binding molecule comprises or consists of avimers.

As used herein, the term “variable region” or “variable domain” is used interchangeably and are common in the art. The variable region typically refers to a portion of an antibody, generally, a portion of a light or heavy chain, typically about the amino-terminal 110 to 120 amino acids in the mature heavy chain and about 90 to 115 amino acids in the mature light chain, which differ extensively in sequence among antibodies and are used in the binding and specificity of a particular antibody for its particular antigen. The variability in sequence is concentrated in those regions called complementarity determining regions (CDRs) while the more highly conserved regions in the variable domain are called framework regions (FR). Without wishing to be bound by any particular mechanism or theory, it is believed that the CDRs of the light and heavy chains are primarily responsible for the interaction and specificity of the antibody with antigen. In certain embodiments, the variable region is a human variable region. In certain embodiments, the variable region comprises rodent or murine CDRs and human framework regions (FRs). In particular embodiments, the variable region is a primate (e.g., non-human primate) variable region. In certain embodiments, the variable region comprises rodent or murine CDRs and primate (e.g., non-human primate) framework regions (FRs).

As used herein, an antigen binding molecule, an antibody, or an antigen binding molecule thereof “cross-competes” with a reference antibody or an antigen binding molecule thereof if the interaction between an antigen and the first binding molecule, an antibody, or an antigen binding molecule thereof blocks, limits, inhibits, or otherwise reduces the ability of the reference binding molecule, reference antibody, or an antigen binding molecule thereof to interact with the antigen. Cross competition may be complete, e.g., binding of the binding molecule to the antigen completely blocks the ability of the reference binding molecule to bind the antigen, or it may be partial, e.g., binding of the binding molecule to the antigen reduces the ability of the reference binding molecule to bind the antigen. In certain embodiments, an antigen binding molecule that cross-competes with a reference antigen binding molecule binds the same or an overlapping epitope as the reference antigen binding molecule. In other embodiments, the antigen binding molecule that cross-competes with a reference antigen binding molecule binds a different epitope as the reference antigen binding molecule. Numerous types of competitive binding assays may be used to determine if one antigen binding molecule competes with another, for example: solid phase direct or indirect radioimmunoassay (RIA); solid phase direct or indirect enzyme immunoassay (EIA); sandwich competition assay (Stahli et al., 1983, Methods in Enzymology 9:242-253); solid phase direct biotin-avidin EIA (Kirkland et al., 1986, J. Immunol. 137:3614-3619); solid phase direct labeled assay, solid phase direct labeled sandwich assay (Harlow and Lane, 1988, Antibodies, A Laboratory Manual, Cold Spring Harbor Press); solid phase direct label RIA using 1-125 label (Morel et al., 1988, Molec. Immunol. 25:7-15), solid phase direct biotin-avidin EIA (Cheung, et al., 1990, Virology 176:546-552), and direct labeled RIA (Moldenhauer et al., 1990, Scand. J. Immunol. 32:77-82).

An “antigen” refers to any molecule that provokes an immune response or is capable of being bound by an antibody or an antigen binding molecule. The immune response may involve either antibody production, or the activation of specific immunologically-competent cells, or both. A person of skill in the art would readily understand that any macromolecule, including virtually all proteins or peptides, could serve as an antigen. An antigen may be endogenously expressed, i.e. expressed by genomic DNA, or may be recombinantly expressed. An antigen may be specific to a certain tissue, such as a cancer cell, or it may be broadly expressed. In addition, fragments of larger molecules may act as antigens. In one embodiment, antigens are tumor antigens.

The term “allogeneic” refers to any material derived from one individual, which is then introduced to another individual of the same species, e.g., allogeneic T cell transplantation.

The terms “transduction” and “transduced” refer to the process whereby foreign DNA is introduced into a cell via viral vector (see Jones et al., “Genetics: principles and analysis,” Boston: Jones & Bartlett Publ. (1998)). In some embodiments, the vector is a retroviral vector, a DNA vector, a RNA vector, an adenoviral vector, a baculoviral vector, an Epstein Barr viral vector, a papovaviral vector, a vaccinia viral vector, a herpes simplex viral vector, an adenovirus associated vector, a lentiviral vector, or any combination thereof.

A “cancer” refers to a broad group of various diseases characterized by the uncontrolled growth of abnormal cells in the body. Unregulated cell division and growth results in the formation of malignant tumors that invade neighboring tissues and may also metastasize to distant parts of the body through the lymphatic system or bloodstream. A “cancer” or “cancer tissue” may include a tumor. Examples of cancers that may be treated by the methods of the present invention include, but are not limited to, cancers of the immune system including lymphoma, leukemia, myeloma, and other leukocyte malignancies. In some embodiments, the methods of the present invention may be used to reduce the tumor size of a tumor derived from, for example, bone cancer, pancreatic cancer, skin cancer, cancer of the head or neck, cutaneous or intraocular malignant melanoma, uterine cancer, ovarian cancer, rectal cancer, cancer of the anal region, stomach cancer, testicular cancer, uterine cancer, carcinoma of the fallopian tubes, carcinoma of the endometrium, carcinoma of the cervix, carcinoma of the vagina, carcinoma of the vulva, multiple myeloma, Hodgkin's Disease, non-Hodgkin's lymphoma (NHL), primary mediastinal large B cell lymphoma (PMBC), diffuse large B cell lymphoma (DLBCL), follicular lymphoma (FL), transformed follicular lymphoma, splenic marginal zone lymphoma (SMZL), cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, chronic or acute leukemia, acute myeloid leukemia, chronic myeloid leukemia, acute lymphoblastic leukemia (ALL) (including non T cell ALL), chronic lymphocytic leukemia (CLL), solid tumors of childhood, lymphocytic lymphoma, cancer of the bladder, cancer of the kidney or ureter, carcinoma of the renal pelvis, neoplasm of the central nervous system (CNS), primary CNS lymphoma, tumor angiogenesis, spinal axis tumor, brain stem glioma, pituitary adenoma, Kaposi's sarcoma, epidermoid cancer, squamous cell cancer, T-cell lymphoma, environmentally induced cancers including those induced by asbestos, other B cell malignancies, and combinations of said cancers. In one particular embodiment, the cancer is multiple myeloma. The particular cancer may be responsive to chemo- or radiation therapy or the cancer may be refractory. A refractor cancer refers to a cancer that is not amendable to surgical intervention and the cancer is either initially unresponsive to chemo- or radiation therapy or the cancer becomes unresponsive over time.

Additional examples of cancers that may be treated by the methods of the present invention include, relapsed or refractory large B-cell lymphoma, diffuse large B-cell lymphoma (DLBCL) not otherwise specified, primary mediastinal large B-cell lymphoma, high grade B-cell lymphoma, or DLBCL arising from follicular lymphoma.

An “anti-tumor effect” as used herein, refers to a biological effect that may present as a decrease in tumor volume, a decrease in the number of tumor cells, a decrease in tumor cell proliferation, a decrease in the number of metastases, an increase in overall or progression-free survival, an increase in life expectancy, or amelioration of various physiological symptoms associated with the tumor. An anti-tumor effect may also refer to the prevention of the occurrence of a tumor, e.g., a vaccine.

A “cytokine,” as used herein, refers to a non-antibody protein that is released by one cell in response to contact with a specific antigen, wherein the cytokine interacts with a second cell to mediate a response in the second cell. A cytokine may be endogenously expressed by a cell or administered to a subject. Cytokines may be released by immune cells, including macrophages, B cells, T cells, and mast cells to propagate an immune response. Cytokines may induce various responses in the recipient cell. Cytokines may include homeostatic cytokines, chemokines, pro-inflammatory cytokines, effectors, and acute-phase proteins. For example, homeostatic cytokines, including interleukin (IL) 7 and IL-15, promote immune cell survival and proliferation, and pro-inflammatory cytokines may promote an inflammatory response. Examples of homeostatic cytokines include, but are not limited to, IL-2, IL-4, IL-5, IL-7, IL-10, IL-12p40, IL-12p70, IL-15, and interferon (IFN) gamma. Examples of pro-inflammatory cytokines include, but are not limited to, IL-1a, IL-1b, IL-6, IL-13, IL-17a, tumor necrosis factor (TNF)-alpha, TNF-beta, fibroblast growth factor (FGF) 2, granulocyte macrophage colony-stimulating factor (GM-CSF), soluble intercellular adhesion molecule 1 (sICAM-1), soluble vascular adhesion molecule 1 (sVCAM-1), vascular endothelial growth factor (VEGF), VEGF-C, VEGF-D, and placental growth factor (PLGF). Examples of effectors include, but are not limited to, granzyme A, granzyme B, soluble Fas ligand (sFasL), and perforin. Examples of acute phase-proteins include, but are not limited to, C-reactive protein (CRP) and serum amyloid A (SAA).

“Chemokines” are a type of cytokine that mediates cell chemotaxis, or directional movement. Examples of chemokines include, but are not limited to, IL-8, IL-16, eotaxin, eotaxin-3, macrophage-derived chemokine (MDC or CCL22), monocyte chemotactic protein 1 (MCP-1 or CCL2), MCP-4, macrophage inflammatory protein 1α (MIP-1α, MIP-1a), MIP-1β (MIP-1b), gamma-induced protein 10 (IP-10), and thymus and activation regulated chemokine (TARC or CCL17).

A “therapeutically effective amount,” “effective dose,” “effective amount,” or “therapeutically effective dosage” of a therapeutic agent, e.g., engineered CAR T cells, is any amount that, when used alone or in combination with another therapeutic agent, protects a subject against the onset of a disease or promotes disease regression evidenced by a decrease in severity of disease symptoms, an increase in frequency and duration of disease symptom-free periods, or a prevention of impairment or disability due to the disease affliction. The ability of a therapeutic agent to promote disease regression may be evaluated using a variety of methods known to the skilled practitioner, such as in human subjects during clinical trials, in animal model systems predictive of efficacy in humans, or by assaying the activity of the agent in in vitro assays.

The term “lymphocyte” as used herein includes natural killer (NK) cells, T cells, or B cells. NK cells are a type of cytotoxic (cell toxic) lymphocyte that represent a major component of the inherent immune system. NK cells reject tumors and cells infected by viruses. It works through the process of apoptosis or programmed cell death. They were termed “natural killers” because they do not require activation in order to kill cells. T-cells play a major role in cell-mediated-immunity (no antibody involvement). Its T-cell receptors (TCR) differentiate themselves from other lymphocyte types. The thymus, a specialized organ of the immune system, is primarily responsible for the T cell's maturation. There are six types of T-cells, namely: Helper T-cells (e.g., CD4+ cells), Cytotoxic T-cells (also known as TC, cytotoxic T lymphocyte, CTL, T-killer cell, cytolytic T cell, CD8+ T-cells or killer T cell), Memory T-cells ((i) stem memory T_{SCM} cells, like naive cells, are CD45RO−, CCR7+, CD45RA+, CD62L+ (L-selectin), CD27+, CD28+ and IL-7Rα+, but they also express large amounts of CD95, IL-2Rβ, CXCR3, and LFA-1, and show numerous functional attributes distinctive of memory cells); (ii) central memory T_{CM} cells express L-selectin and the CCR7, they secrete IL-2, but not IFNγ or IL-4, and (iii) effector memory T_{EM} cells, however, do not express L-selectin or CCR7 but produce effector cytokines like IFNγ and IL-4), Regulatory T-cells (Tregs, suppressor T cells, or CD4+CD25+ regulatory T cells), Natural Killer T-cells (NKT) and Gamma Delta T-cells. B-cells, on the other hand, play a principal role in humoral immunity (with antibody involvement). They make antibodies and antigens, perform the role of antigen-presenting cells (APCs), and turn into memory B-cells after activation by antigen interaction. In mammals, immature B-cells are formed in the bone marrow.

The term “genetically engineered”, “engineered”, or “modified” refers to a method of modifying a cell, including, but not limited to, creating a deficiency in a gene by deleting a coding or non-coding region or a portion thereof or by antisense technology, or increasing expression of a protein introducing a coding region or a portion thereof. In some embodiments, the cell that is modified is a stem cell (e.g., hematopoietic stem cell (HSC), embryonic stem cell (ES), induced pluripotent stem (iPS) cell), lymphocyte (e.g., a T cell), which may be obtained either from a patient or a donor. The cell may be modified to express an exogenous construct, such as, e.g., a pre-TCRα protein, a chimeric antigen receptor (CAR) or a T cell receptor (TCR), which may be incorporated into the cell's genome.

An “immune response” refers to the action of a cell of the immune system (for example, T lymphocytes, B lymphocytes, natural killer (NK) cells, macrophages, eosinophils, mast cells, dendritic cells and neutrophils) and soluble macromolecules produced by any of these cells or the liver (including Abs, cytokines, and complement) that results in selective targeting, binding to, damage to, destruction of, and/or elimination from a vertebrate's body of invading pathogens, cells or tissues infected with pathogens, cancerous or other abnormal cells, or, in cases of autoimmunity or pathological inflammation, normal human cells or tissues.

The term “immunotherapy” refers to the treatment of a subject afflicted with, or at risk of contracting or suffering a recurrence of, a disease by a method comprising inducing, enhancing, suppressing, or otherwise modifying an immune response. Examples of immunotherapy include, but are not limited to, T cell therapies. T cell therapy may include adoptive T cell therapy, tumor-infiltrating lymphocyte (TIL) immunotherapy, autologous cell therapy, engineered autologous cell therapy (eACT™), and allogeneic T cell transplantation. However, one of skill in the art would recognize that the conditioning methods disclosed herein would enhance the effectiveness of any transplanted T cell therapy. Examples of T cell therapies are described in U.S. Patent Publication Nos. 2014/0154228 and 2002/0006409, U.S. Pat. No. 5,728,388, and International Publication No. WO 2008/081035.

The T cells of the immunotherapy may come from any source known in the art. For example, T cells may be differentiated in vitro from a hematopoietic stem cell population; induced pluripotent stem cells (iPS), embryonic stem cells (ES), or T cells may be obtained from a subject. T cells may be obtained from, e.g., peripheral blood mononuclear cells (PBMCs), bone marrow, lymph node tissue, cord blood, thymus tissue, tissue from a site of infection, ascites, pleural effusion, spleen tissue, and tumors. In addition, the T cells may be derived from one or more T cell lines available in the art. T cells may also be obtained from a unit of blood collected from a subject using any number of techniques known to the skilled artisan, such as FICOLL™ separation and/or apheresis. Additional methods of isolating T cells for a T cell therapy are disclosed in U.S. Patent Publication No. 2013/0287748, which is herein incorporated by references in its entirety.

The term “engineered Autologous Cell Therapy,” which may be abbreviated as “eACT™,” also known as adoptive cell transfer, is a process by which a patient's own T cells are collected and subsequently genetically altered to recognize and target one or more antigens expressed on the cell surface of one or more specific tumor cells or malignancies. T cells may be engineered to express, for example, chimeric antigen receptors (CAR) or T cell receptor (TCR). CAR positive (+) T cells are engineered to express an extracellular single chain variable fragment (scFv) with specificity for a particular tumor antigen linked to an intracellular signaling part comprising at least one costimulatory domain and at least one activating domain. The costimulatory domain may be derived from a naturally-occurring costimulatory domain, or a variant thereof, e.g., a variant having a truncated hinge domain (“THD”), and the activating domain may be derived from, e.g., CD3-zeta. In certain embodiments, the CAR is designed to have two, three, four, or more costimulatory domains. The CAR scFv may be designed to target, for example, CD19, which is a transmembrane protein expressed by cells in the B cell lineage, including all normal B cells and B cell malignances, including but not limited to NHL, CLL, and non T cell ALL. In some embodiments, the CAR is engineered such that the costimulatory domain is expressed as a separate polypeptide chain. Example CAR T cell therapies and constructs are described in U.S. Patent Publication Nos. 2013/0287748, 2014/0227237, 2014/0099309, and 2014/0050708, and these references are incorporated by reference in their entirety.

A “patient” as used herein includes any human who is afflicted with a cancer (e.g., a lymphoma or leukemia). The terms “subject” and “patient” are used interchangeably herein.

As used herein, the term “in vitro cell” refers to any cell, which is cultured ex vivo. In particular, an in vitro cell may include a T cell.

The terms “peptide,” “polypeptide,” and “protein” are used interchangeably, and refer to a compound comprised of amino acid residues covalently linked by peptide bonds. A protein or peptide contains at least two amino acids, and no limitation is placed on the maximum number of amino acids that may comprise a protein or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

“Stimulation,” as used herein, refers to a primary response induced by binding of a stimulatory molecule with its cognate ligand, wherein the binding mediates a signal transduction event. A “stimulatory molecule” is a molecule on a T cell, e.g., the T cell receptor (TCR)/CD3 complex that specifically binds with a cognate stimulatory ligand present on an antigen present cell. A “stimulatory ligand” is a ligand that when present on an antigen presenting cell (e.g., an APC, a dendritic cell, a B-cell, and the like) may specifically bind with a stimulatory molecule on a T cell, thereby mediating a primary response by the T cell, including, but not limited to, activation, initiation of an immune response, proliferation, and the like. Stimulatory ligands include, but are not limited to, an anti-CD3 antibody, an MHC Class I molecule loaded with a peptide, a superagonist anti-CD2 antibody, and a superagonist anti-CD28 antibody.

A “costimulatory signal,” as used herein, refers to a signal, which in combination with a primary signal, such as TCR/CD3 ligation, leads to a T cell response, such as, but not limited to, proliferation and/or upregulation or down regulation of key molecules.

A “costimulatory ligand” as used herein, includes a molecule on an antigen presenting cell that specifically binds a cognate co-stimulatory molecule on a T cell. Binding of the costimulatory ligand provides a signal that mediates a T cell response, including, but not limited to, proliferation, activation, differentiation, and the like. A costimulatory ligand induces a signal that is in addition to the primary signal provided by a stimulatory molecule, for instance, by binding of a T cell receptor (TCR)/CD3 complex with a major histocompatibility complex (MHC) molecule loaded with peptide. A co-stimulatory ligand may include, but is not limited to, 3/TR6, 4-1BB ligand, agonist or antibody that binds Toll ligand receptor, B7-1 (CD80), B7-2 (CD86), CD30 ligand, CD40, CD7, CD70, CD83, herpes virus entry mediator (HVEM), human leukocyte antigen G (HLA-G), ILT4, immunoglobulin-like transcript (ILT) 3, inducible costimulatory ligand (ICOS-L), intercellular adhesion molecule (ICAM), ligand that specifically binds with B7-H3, lymphotoxin beta receptor, MHC class I chain-related protein A (MICA), MHC class I chain-related protein B (MICB), OX40 ligand, PD-L2, or programmed death (PD) L1. A co-stimulatory ligand includes, without limitation, an antibody that specifically binds with a co-stimulatory molecule present on a T cell, such as, but not limited to, 4-1BB, B7-H3, CD2, CD27, CD28, CD30, CD40, CD7, ICOS, ligand that specifically binds with CD83, lymphocyte function-associated antigen-1 (LFA-1), natural killer cell receptor C (NKG2C), OX40, PD-1, or tumor necrosis factor superfamily member 14 (TNFSF14 or LIGHT).

A “costimulatory molecule” is a cognate binding partner on a T cell that specifically binds with a costimulatory ligand, thereby mediating a costimulatory response by the T cell, such as, but not limited to, proliferation. Costimulatory molecules include, but are not limited to, A “costimulatory molecule” is a cognate binding partner on a T cell that specifically binds with a costimulatory ligand, thereby mediating a costimulatory response by the T cell, such as, but not limited to, proliferation. Costimulatory molecules include, but are not limited to, 4-1BB/CD137, B7-H3, BAFFR, BLAME (SLAMF8), BTLA, CD 33, CD 45, CD100 (SEMA4D), CD103, CD134, CD137, CD154, CD16, CD160 (BY55), CD18, CD19, CD19a, CD2, CD22, CD247, CD27, CD276 (B7-H3), CD28, CD29, CD3 (alpha; beta; delta; epsilon; gamma; zeta), CD30, CD37, CD4, CD4, CD40, CD49a, CD49D, CD49f, CD5, CD64, CD69, CD7, CD80, CD83 ligand, CD84, CD86, CD8alpha, CD8beta, CD9, CD96 (Tactile), CDl-la, CDl-lb, CDl-lc, CDl-ld, CDS, CEACAM1, CRT AM, DAP-10, DNAM1 (CD226), Fc gamma receptor, GADS, GITR, HVEM (LIGHTR), IA4, ICAM-1, ICAM-1, ICOS, Ig alpha (CD79a), IL2R beta, IL2R gamma, IL7R alpha, integrin, ITGA4, ITGA4, ITGA6, ITGAD, ITGAE, ITGAL, ITGAM, ITGAX, ITGB2, ITGB7, ITGB1, KIRDS2, LAT, LFA-1, LFA-1, LIGHT, LIGHT (tumor necrosis factor superfamily member 14; TNFSF14), LTBR, Ly9 (CD229), lymphocyte function-associated antigen-1 (LFA-1 (CDl la/CD18), MHC class I molecule, NKG2C, NKG2D, NKp30, NKp44, NKp46, NKp80 (KLRF1), OX40, PAG/Cbp, PD-1, PSGL1, SELPLG (CD162), signaling lymphocytic activation molecule, SLAM (SLAMF1; CD150; IPO-3), SLAMF4 (CD244; 2B4), SLAMF6 (NTB-A; Lyl08), SLAMF7, SLP-76, TNF, TNFr, TNFR2, Toll ligand receptor, TRANCE/RANKL, VLA1, or VLA-6, or fragments, truncations, or combinations thereof.

The terms “reducing” and “decreasing” are used interchangeably herein and indicate any change that is less than the original. “Reducing” and “decreasing” are relative terms, requiring a comparison between pre- and post-measurements. “Reducing” and “decreasing” include complete depletions.

“Treatment” or “treating” of a subject refers to any type of intervention or process performed on, or the administration of an active agent to, the subject with the objective of reversing, alleviating, ameliorating, inhibiting, slowing down or preventing the onset, progression, development, severity, or recurrence of a symptom, complication or condition, or biochemical indicia associated with a disease. In one embodiment, “treatment” or “treating” includes a partial remission. In another embodiment, “treatment” or “treating” includes a complete remission.

As used herein, a “TCR proxy” is a molecule (e.g., a peptide, a protein, a synthetic molecule, etc.) that initiates downstream signaling elements that allow or facilitate the development of a T cell from a stem cell in the absence of an endogenous TCR and/or pre-TCR. In some embodiments, the TCR proxy is a defined TCR, a preTCR, a pTa monomer, a pTa/TCRβ heterodimer, a TCRα molecule, a TCRβ molecule, a TCR gamma molecule, a TCR delta molecule, a TCRα/beta heterodimer, a TCR gamma/delta heterodimer, any homodimer of the previous molecules, a TCR like molecule, or other molecule that initiates a TCR signal to allow T cell development. In some embodiments, a TCR proxy comprises one or more molecules (e.g., one, two, three, four, five, six or more molecules). In some embodiments, the one or more molecules are proteins. In some embodiments, the TCR proxy is a protein complex.

As used herein, the term “selectable” means a molecule capable of being targeted by an antibody. In some embodiments, a selectable surface marker is molecule expressed on the surface that is capable of being targeted by an antigen binding molecule (e.g., an antibody).

To calculate percent identity, the sequences being compared are typically aligned in a way that gives the largest match between the sequences. One example of a computer program that may be used to determine percent identity is the GCG program package, which includes GAP (Devereux et al., (1984), Nucl. Acid Res. 12:387; Genetics Computer Group, University of Wisconsin, Madison, Wis.). The computer algorithm GAP is used to align the two polypeptides or polynucleotides for which the percent sequence identity is to be determined. The sequences are aligned for optimal matching of their respective amino acid or nucleotide (the “matched span,” as determined by the algorithm.) In certain embodiments, a standard comparison matrix (see, Dayhoff et al., 1978, Atlas of Protein Sequence and Structure 5:345-352 for the PAM 250 comparison matrix; Henikoff et al., 1992, Proc. Natl. Acad. Sci. U.S.A. 89:10915-10919 for the BLOSUM 62 comparison matrix) is also used by the algorithm.

Various aspects of the invention are described in further detail in the following subsections.

### DETAILED DESCRIPTION

The present disclosure provides, among other things, a modified pluripotent stem cell, genetically engineered stem cells, and their derivatives that efficiently differentiate into T cells and methods of making and using the same. In particular, the present disclosure provides the production of stem cells which may be used in an autologous or allogeneic setting for engineered immunotherapy. When used in cell based immunotherapy, modified pluripotent stem cells described herein may reduce or eliminate the risk of Graft versus Host Disease (GVHD), provide resistance to elimination by a recipient's T cells and NK cells, and allow for controllable T cell activity (e.g., engineered to comprise a suicide gene or kill switch).

T cell responses from adoptive cell therapy may be mediated by T-cells from the recipient. Graft rejection may arise from immunogenicity to the exogenous transgene, reactivity against mismatched Human Histocompatibility Antigen (HLA) (unrelated/haploidentical), or reactivity against minor histocompatibility antigens (MiHA) (e.g., HA-1, HA-2, etc.) (related/unrelated HLA matched/haploidentical). Responses may also be mediated by the donor T-cells leading to GVHD from reactivity against mismatched HLA/MiHA and anti-tumor events from reactivity against tumor antigens/tumor associated MiHA.

To prevent host immune reactivity to cell therapy, (e.g., GVHD induced by mismatched HLA or MiHA), in one aspect, the present disclosure provides a modified pluripotent stem cell engineered to eliminate endogenous TCR expression. In some embodiments, gene editing of endogenous TCR is engineered by knock out (KO) of TCRα and/or TCRβ (TRAC and/or TRBC1/TRBC2). In some embodiments, cells are engineered by KO of RAG1/RAG2 (depending on cell source and differentiation status).

To prevent graft rejection, the present disclosure provides a modified pluripotent stem cell engineered to block expression of donor HLA and/or re-introduce 1 HLA-Class I “non-polymorphic” allele to prevent NK killing (e.g., single chain HLA-E). In some embodiments, modifications are made to HLA Class I molecules (e.g., B-2-microglobulin, individual HLA molecules (HLA-A,-B,-C,-E,-G), TAP1, TAP2 and/or genes associated with Bare Lymphocyte Syndrome I (BLSI)). In some embodiments, modifications are made to HLA Class II molecules (e.g., Transcription factors (RFXANK or RFX5 or RFXAP) or transactivators (CIITA), Genes associated with BLS II, and/or individual HLA molecules (HLA-DP,-DQ-DR,-DM,-DO -alpha and beta chains)). In some embodiments, modifications are made to promote tumor reactivity (e.g., introducing a tumor specific TCR or CAR). In some embodiments, cells are further modified to eliminate inhibitory receptors (e.g., PDCD1, CTLA4). In some embodiments, cells are modified to introduce costimulatory receptors (e.g., CD28, TNFRSF9).

### Pluripotent Stem Cells

Various pluripotent stems cells may be used to practice the present invention. For example, hematopoietic stem cells (HSC) in the bone marrow (also cord blood or peripheral blood) give rise, in addition to all other mature blood cells, to committed thymic progenitors. These thymic progenitors traffic to the thymus where they begin their development to mature T cells. The signaling of Notch receptors via their ligands Delta and Jagged, particularly Notch1 and Delta like 4 in the thymus, drives a transcriptional cascade (i.e. Tcf7, Gata3, Bcl11b, etc.) that results in the rearrangement of TCR loci by the recombinase activating genes RAG1 and RAG2. First, a productive TCRβ rearrangement (i.e. resulting in a TCR protein) will generate a protein that pairs with pTa and traffics to the surface. This surface trafficking conveys a signal back to the cell that allows it to proceed to further development. The surface pTa-TCRβ need not interact with MHC as occurs in a mature TCR—the survival signal may be peptide:MHC independent. The cell then proceeds to rearrange TCRα, is scrutinized for successful alpha/beta pairing, weak recognition of self-peptide:MHC (i.e. positive and negative selection or central tolerance) before becoming a mature naïve T cell and circulating to the periphery. T cells that fail to generate a productive TCRβ and/or TCRα will not express a surface TCR complex, will not receive signals that instruct the cell to continue development or maturation, and ultimately die. As described herein, pluripotent stem cells are modified to regulate T cell responses and control differentiation.

In some embodiments, embryonic stem (ES) or induced pluripotent stem (iPS) cells may be used. Suitable HSCs, ES cells, iPS cells and other stems cells may be cultivated immortal cell lines or isolated directly from a patient. Various methods for isolating, developing, and/or cultivating stem cells are known in the art and may be used to practice the present invention.

In some embodiments, the stem cell is an induced pluripotent stem cell (iPSC) generated from a reprogrammed T-cell. As described herein, the stem cell derived T cell may be used in an autologous or allogeneic setting for engineered immunotherapy.

In some embodiments, the source material may be an induced pluripotent stem cell (iPSC) derived from a T cell or non-T cell. The source material may be an embryonic stem cell. The source material may be a B cell, or any other cell from peripheral blood mononuclear cell isolates, hematopoietic progenitor, hematopoietic stem cell, mesenchymal stem cell, adipose stem cell, or any other somatic cell type.

### Modification of Pluripotent Stem cells

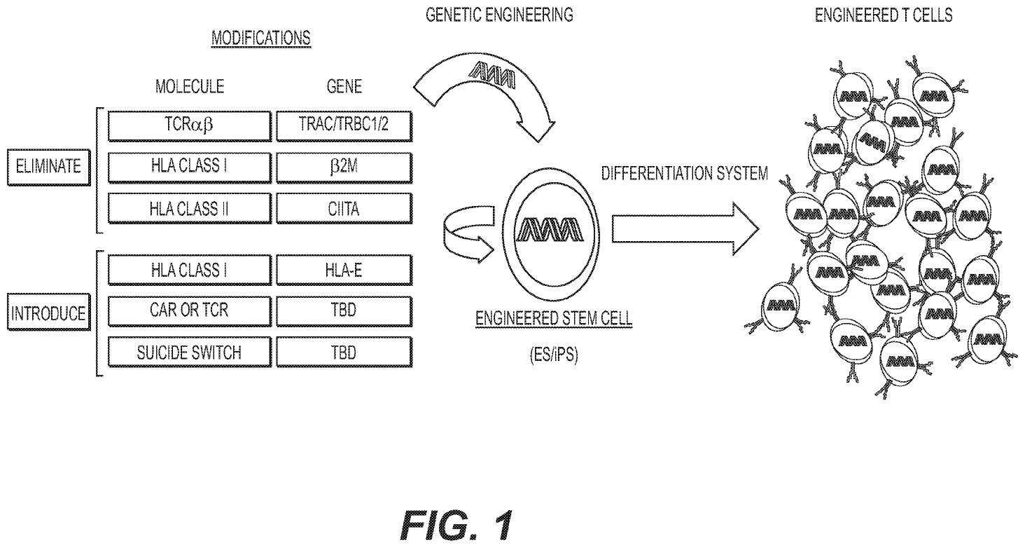

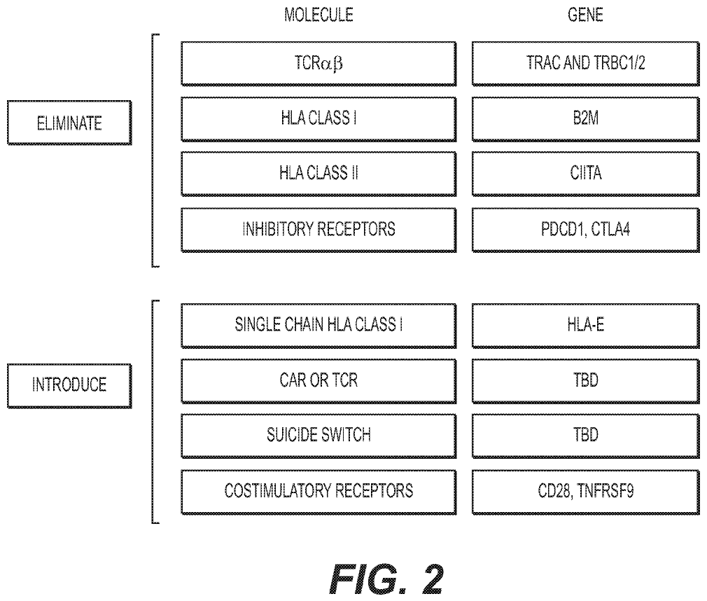

According to the present invention, modification of iPSC or other stem cells (e.g., embryonic stem) may be used to generate a large, perhaps infinite, number of engineered T cells with desired lineage. The present invention generates modified stem cells capable of differentiation to T cells from engineered stem cells. An exemplary modification strategy is shown in FIG. 1 and FIG. 2.

The targeted loci for modification may be determined using a targeting strategy to take advantage of the endogenous promoter, or include an exogenous promoter to drive expression of the antigen receptor. In some embodiments, the targeted locus is the productively rearranged TRAC or TRBC locus of an ab-T-cell using the endogenous promoter. In some embodiments, the locus is the TRGC or TRDC using the endogenous or exogenous promoter.

In some embodiments, the locus is a productive/nonproductive TRAC or TRBC or TRGC or TRDC with exogenous promoter. The targeting strategy may take advantage of one or more of any combination of the productive/nonproductive TRAC or TRBC with or without an exogenous promoter.

According to the present invention, modification of HSC or other stem cells (embryonic stem (ES) or induced pluripotent stem (iPS)) may be used to generate a large, perhaps infinite, number of engineered T cells with desired lineage.

The present invention generates modified stem cells capable of differentiation to T cells from engineered stem cells. In some embodiments, cells are differentiated in the ATO system. The introduction of pre-TCRα (pTa) and/or the knockout of TCRα (TCRα) provide enforced/sustained pTa pairing with TCRβ (TCRβ). The pTa-TCRβ pair provides the necessary signaling for stem cells to develop into mature T cells in the absence of TCRα. The pTa-TCRβ promotes T cell differentiation but lacks reactivity to host peptide:MHC molecules. pTa may be provided naturally by the cell, or provided as an engineered exogenous construct. Stem cells may or may not harbor an engineered CAR or exogenous TCR, antigen receptor, recognizing a target molecule. Target molecule may be expressed on tissue to be eliminated (e.g. cancerous lesion or other) or tissue to induce immune tolerance (e.g., pancreatic islet cell).

pTa-TCRβ is sufficient to drive development (e.g., through ATO) but will not convey any antigen receptor reactivity (i.e. no reactivity of the receptor against MHC, thus no GvHD via the TCRβ). Thus, this method allows the development of T cells with a surface expressed TCR complex, but without MHC reactivity.

Another embodiment of the invention includes the use of a pTa and/or TCRβ that is capable of recognizing antigen independent of the complimentary chain: that is a pTa that may recognize peptide:MHC or other ligand, or TCRβ that may recognize peptide:MHC or other ligand. Peptide:MHC or ligand may be provided naturally or in an engineered state by stem cells, developing thymocytes, mature T cells, co-cultured cell line, stromal cell line in complex with stem cells in ATO, or other differentiation systems. pTa and/or TCRβ may engage this ligand naturally, pTa and/or TCRβ may be modified or engineered to engage this ligand, or ligand may be modified or engineered to engage natural or engineered pTa and/or TCRβ. The resulting effect on development may enhance or hinder cell proliferation, speed up or slow down T cell development, halt T cell development in a particular developmental stage, or direct thymocytes to develop into a particular lineage such as cytotoxic CD8+, helper CD4+ including but not limited to Th1/Th2/Th17, etc., regulatory T cell (Treg), intra epithelial lymphocyte (IEL), alpha-beta T cell, gamma-delta T cell, mature alpha-beta or gamma-delta T cell that is co-receptor independent (i.e. CD4 CD8 double negative) and others.

Another aspect of the invention is directed to a method of making a cell expressing a CAR or a TCR comprising introducing pre-TCRα (pTa) and/or knockout of TCRα (TCRα) to provide enforced or sustained pTa pairing with TCRβ (TCRβ). The pTa-TCRβ pair will provide the necessary signaling for stem cells to develop into mature T cells in the absence of TCRα. pTa may be provided naturally by the cell, or provided as an engineered exogenous construct. Stem cells may or may not harbor an engineered CAR or exogenous TCR, antigen receptor, recognizing a target molecule. Target molecule may be expressed on tissue to be eliminated (e.g. cancerous lesion or other) or tissue to induce immune tolerance (e.g. pancreatic islet cell).

Knockout of specific target loci may be accomplished with an engineered nuclease (TALEN, megaTAL, CRISPR, ZFN, etc.), without a nuclease, by homologous recombination (HR), or other gene modifying method known in the art. Target genes may be edited using CRISPR/Cas9, a zinc finger nucleases (ZFN), a TALEN, a MegaTAL, a meganuclease, Cpf1, homologous recombination, a single stranded oligodeoxynucleotide (ssODN), or other technology.

Genes may be knocked out using technology described above. Genes may be knocked out and left disrupted, or another gene may be knocked into the place of the disrupted gene. The knocked in gene may be designed to be in frame to take advantage of endogenous locus expression. In some embodiments, an exogenous promoter may be incorporated in the donor (knock-in) construct to drive expression.

Stem cells may or may not harbor an engineered CAR or exogenous TCR, antigen receptor, recognizing a target molecule. Target molecule may be expressed on tissue to be eliminated (e.g. cancerous lesion or other) or tissue to induce immune tolerance (e.g. pancreatic islet cell).

Nucleases, HR template, antigen receptor (i.e. CAR or TCR), and exogenous constructs may be delivered by electroporation of DNA or RNA, viral mediated delivery, passive transfer, etc. Constructs may be knocked into an endogenous gene locus taking advantage of innate gene regulatory elements, constitutive physiologic expression level, or contain a defined promoter. The defined promoter may be constitutively active or restricted to distinct stages of cell development and/or cell cycle, etc.

### MHC Related Modifications

In some embodiments, knockout of beta 2 microglobulin may be used to eliminate expression of Class 1a HLA genes to eliminate recognition of the cells by the recipient (host) immune system of the engineered cells.

In some embodiments, reduction or elimination by the host immune system may be achieved by disruption of genes comprising the cellular machinery associated with the processing or loading of peptides into the MHC I or MHC II complexes. Examples include, but are not limited to, calnexin, BiP, calreticulin, ERp57, Tapasin, TAP, ERAAP, or proteins of the proteasome or immunoproteasome.

In some embodiments, knockout of the genes CIITA or RFX5 may be used to achieve reduction or elimination of MHC class II. Targeting of specific individual MHC I and MHC II gene in the target cell may also be employed to reduce or eliminate expression of MHC I and/or MHC II proteins.

### Antigen Receptor Related

in some embodiments, knockout of recombination related genes, e.g., RAG1, RAG2 to prevent recombination of endogenous TCRα, TCRβ, TCRgamma, TCRdelta genes. In some embodiments, RAG1/2 knockout may be used to prevent recombination of the B cell receptor (BCR).

### Gene Addition to Prevent Immune Recognition

To prevent recognition of MHC void cells by NK cells, in some embodiments, the introduction of a semi-invariant HLA-E molecule is used. This molecule may be a native HLA-E sequence, a codon optimized/degenerate modified sequence, a truncated form of HLA-E produced by the removal of one or more amino acid, an elongated form of HLA-E produced by the addition of one or more amino acid to HLA-E. The HLA-E molecule may be a fusion of native HLA-E, or any variant described above, and beta 2 microglobulin. Beta 2 microglobulin may be the native sequence, a codon optimized/degenerate modified sequence, any addition or removal of amino acids, etc. The HLA-E molecule may be a further fusion to include a peptide sequence to bind in the HLA-E molecule, specifically the peptide groove. In some embodiments, a linker between any of the segments of the fusion may be used.

Expression may be driven by incorporation of the HLA-E molecule, in any form, into a gene locus taking advantage of the endogenous promoter to drive expression. Alternatively the construct may harbor an exogenous promoter to drive expression.

### Control/Elimination of Cellular Product

A gene known as a suicide gene may be incorporated into the cellular product. The purpose of this gene is to allow the elimination of gene modified cells in the case of an adverse event, self-reactivity of infused cells, eradication of cancer, or other. In some embodiments, the suicide gene is introduced to a random genomic position, or a targeted locus (e.g., a metabolic gene locus, DNA/RNA replication gene locus, etc.)

The suicide gene may be driven by an exogenous promoter, or take advantage of an endogenous promoter of an integrated locus.

In some embodiments, the suicide gene is sr39TK, which allows elimination of cells by the introduction of ganciclovir. This gene may also be used to image gene modified cells using positron emission tomography to localized cells in the recipient/host.

The suicide gene may also be a chemically induced caspase, dimerization induced by a small molecule/chemically induced dimerizer (CID). The suicide gene may also be a selectable surface marker (CD19 or CD20 or CD34 or EGFR or LNGFR, etc) allowing the cells to be eliminated by introduction of an antibody through antibody dependent cellular cytotoxicity, complement cascade, etc.

In the case of a selectable marker, this may be used to enrich for gene modified cells by magnetic bead bound antibody, sorting by flow cytometry, activation through antibody, etc.

The gene modified cells may be generated as single cell clones. In some embodiments, cells may be derived from a population.

The genome, in whole or in part, may be sequenced to identify and/or verify the location of integrations. Sequencing may also be employed to identify changes in the genome of the cell line during the generation of the final cell product, the master cell bank, etc.

### TCR Proxy

Developing T-lymphocytes undergo genomic rearrangement of their alpha and beta T cell receptor (TCR) loci, generating a unique heterodimeric TCR exclusive to each cell. These TCRs may recognize any antigen presented as a peptide loaded into the major histocompatibility complex (MHC) on another cell. Two distinct stages in thymic development ensure that the body is populated by functional immune cells that are capable of interacting with self-MHC (positive selection) but do not recognize healthy self-antigens (negative selection). These stages are mediated by a signal that is generated by the interaction between the TCR and MHC. If no signal is generated (e.g., TCR cannot bind self-MHC, thus the immune cell does not provide protection) the cell undergoes a process known as “death by neglect.” If a strong signal is generated (e.g., TCR binds strongly to self-MHC) the cell undergoes apoptosis.

In some embodiments, universal allogenic T cell immunotherapy (allo), as described herein, comprises a cell that is edited or deleted for the TRAC and/or TRBC loci to prevent undesirable reactivity against the recipient host. In the context of stem-cell derived allo, the loss of either gene will result in the partial development of a thymocyte, but not a fully mature naïve T-cell. Knockout of TRAC will result in a cell stalled at the CD4CD8 double positive stage (e.g., a functional TCR-beta gene that may pair with endogenous pre-TCR-alpha (pTa)). Knockout of TRBC will result in a cell halted at the double negative (DN) stage (never getting a TCR signal).

In some embodiments, a cell may be engineered to introduce a TCR proxy. As used herein, a TCR proxy is a molecule (e.g., a protein) that initiates downstream signaling elements that will allow or facilitate the development of a T cell from a stem cell in the absence of an endogenous TCR and/or pre-TCR. In some embodiments, the TCR proxy is a defined TCR, a preTCR, a pTa monomer, a pTa/TCRβ heterodimer, a TCRα molecule, a TCRβ molecule, a TCR gamma molecule, a TCR delta molecule, a TCRα/beta heterodimer, a TCR gamma/delta heterodimer, any homodimer of the previous molecules, a TCR like molecule, or other molecule that initiates a TCR signal to allow T cell development.

In some embodiments, the TCR proxy is expressed in a non-gene edited cell where it suppresses the rearrangement and/or expression of the endogenous loci (allelic exclusion). In some embodiments, in a cell edited to lack an endogenous TCR, the TCR proxy initiates positive survival signals to drive development to a mature naïve T cell. In some embodiments, the TCR proxy is a TCR cloned from a peripheral T cell, reactive against a known peptide-MHC (such as viral antigen reactive TCRs, cancer/testes antigen reactive TCRs, etc.). In some embodiments, the TCR proxy is a chimeric molecule such as pTa and TCRβ.

In some embodiments, the TCR proxy is a subcomponent of the TCR that initiates a downstream TCR signal (e.g., CD3 chains). In some embodiments, the TCR proxy is a completely synthetic molecule that provides TCR signals to the T cell.

In some embodiments, the TCR proxy is the therapeutic TCR (e.g., the TCR that will engage tumor antigen when expressed in T cells). In some embodiments, the TCR proxy is not the therapeutic TCR (e.g., the TCR that will engage tumor antigen when expressed in T cells).

In some embodiments, the TCR proxy is chimeric, murine, and/or an engineered version of a therapeutic TCR. In some embodiments, the TCR proxy is a non alloreactive alpha/beta or gamma/delta TCR, a pre-TCR plus/minus one of the other TCR chains, single chain TCR chimeras, engineered TCR variants which lack the V domains.

### Chimeric Antigen Receptors and T Cell Receptors

Chimeric antigen receptors (CARs or CAR-Ts) and T cell receptors (TCRs) may be introduced into modified pluripotent stem cells according to the present invention. These engineered receptors may be readily inserted into and expressed by modified pluripotent stem cells in accordance with techniques known in the art. With a CAR, a single receptor may be programmed to both recognize a specific antigen and, when bound to that antigen, activate the immune cell to attack and destroy the cell bearing that antigen. When these antigens exist on tumor cells, an immune cell that expresses the CAR may target and kill the tumor cell. Chimeric antigen receptors incorporate costimulatory (signaling) domains to increase their potency. See U.S. Pat. Nos. 7,741,465, and 6,319,494, as well as Krause et al. and Finney et al. (supra), Song et al., Blood 119:696-706 (2012); Kalos et al., Sci. Transl. Med 3:95 (2011); Porter et al., N. Engl. J. Med 365:725-33 (2011), and Gross et al., Annu. Rev. Pharmacol. Toxicol. 56:59-83 (2016).

In some embodiments, a costimulatory domain which includes a truncated hinge domain (“THD”) further comprises some or all of a member of the immunoglobulin family such as IgG1, IgG2, IgG3, IgG4, IgA, IgD, IgE, IgM, or fragment thereof.

In some embodiments, the THD is derived from a human complete hinge domain (“CHD”). In other embodiments, the THD is derived from a rodent, murine, or primate (e.g., non-human primate) CHD of a costimulatory protein. In some embodiments, the THD is derived from a chimeric CHD of a costimulatory protein.

The costimulatory domain for the CAR or TCR of the invention may further comprise a transmembrane domain and/or an intracellular signaling domain. The transmembrane domain may be designed to be fused to the extracellular domain of the CAR. It may similarly be fused to the intracellular domain of the CAR. In some embodiments, the transmembrane domain that naturally is associated with one of the domains in a CAR is used. In some instances, the transmembrane domain may be selected or modified by amino acid substitution to avoid binding of such domains to the transmembrane domains of the same or different surface membrane proteins to minimize interactions with other members of the receptor complex. The transmembrane domain may be derived either from a natural or from a synthetic source. Where the source is natural, the domain may be derived from any membrane-bound or transmembrane protein. Transmembrane regions of particular use in this invention may be derived from (i.e., comprise) 4-1BB/CD137, activating NK cell receptors, an Immunoglobulin protein, B7-H3, BAFFR, BLAME (SLAMF8), BTLA, CD100 (SEMA4D), CD103, CD160 (BY55), CD18, CD19, CD19a, CD2, CD247, CD27, CD276 (B7-H3), CD28, CD29, CD3 delta, CD3 epsilon, CD3 gamma, CD3 zeta, CD30, CD4, CD40, CD49a, CD49D, CD49f, CD69, CD7, CD84, CD8, CD8alpha, CD8beta, CD96 (Tactile), CDl la, CDl lb, CDl lc, CDl ld, CDS, CEACAM1, CRT AM, cytokine receptor, DAP-10, DNAM1 (CD226), Fc gamma receptor, GADS, GITR, HVEM (LIGHTR), IA4, ICAM-1, ICAM-1, Ig alpha (CD79a), IL-2R beta, IL-2R gamma, IL-7R alpha, inducible T cell costimulator (ICOS), integrins, ITGA4, ITGA4, ITGA6, ITGAD, ITGAE, ITGAL, ITGAM, ITGAX, ITGB2, ITGB7, ITGB1, KIRDS2, LAT, LFA-1, LFA-1, a ligand that specifically binds with CD83, LIGHT, LIGHT, LTBR, Ly9 (CD229), lymphocyte function-associated antigen-1 (LFA-1; CDl-la/CD18), MHC class 1 molecule, NKG2C, NKG2D, NKp30, NKp44, NKp46, NKp80 (KLRF1), OX-40, PAG/Cbp, programmed death-1 (PD-1), PSGL1, SELPLG (CD162), Signaling Lymphocytic Activation Molecules (SLAM proteins), SLAM (SLAMF1; CD150; IPO-3), SLAMF4 (CD244; 2B4), SLAMF6 (NTB-A; Lyl08), SLAMF7, SLP-76, TNF receptor proteins, TNFR2, TNFSF14, a Toll ligand receptor, TRANCE/RANKL, VLA1, or VLA-6, or a fragment, truncation, or a combination thereof.

Optionally, short linkers may form linkages between any or some of the extracellular, transmembrane, and intracellular domains of the CAR. In some embodiments, the linker may be derived from repeats of glycine-glycine-glycine-glycine-serine (SEQ ID NO: 3) (G4S)n or GSTSGSGKPGSGEGSTKG (SEQ ID NO: 2). In some embodiments, the linker comprises 3-20 amino acids and an amino acid sequence at least 80%, 81%, 82%, 83%, 84%, 85%, 86%, 87%, 88%, 89%, 90%, 91%, 92%, 93%, 94%, 95%, 96%, 97%, 98%, 99% or 100% identical to GSTSGSGKPGSGEGSTKG (SEQ ID NO: 2).

The linkers described herein, may also be used as a peptide tag. The linker peptide sequence may be of any appropriate length to connect one or more proteins of interest and is preferably designed to be sufficiently flexible so as to allow the proper folding and/or function and/or activity of one or both of the peptides it connects. Thus, the linker peptide may have a length of no more than 10, no more than 11, no more than 12, no more than 13, no more than 14, no more than 15, no more than 16, no more than 17, no more than 18, no more than 19, or no more than 20 amino acids. In some embodiments, the linker peptide may have a length of at least 3, at least 4, at least 5, at least 6, at least 7, at least 8, at least 9, at least 10, at least 11, at least 12, at least 13, at least 14, at least 15, at least 16, at least 17, at least 18, at least 19, or at least 20 amino acids. In some embodiments, the linker comprises at least 7 and no more than 20 amino acids, at least 7 and no more than 19 amino acids, at least 7 and no more than 18 amino acids, at least 7 and no more than 17 amino acids, at least 7 and no more than 16 amino acids, at least 7 and no more 15 amino acids, at least 7 and no more than 14 amino acids, at least 7 and no more than 13 amino acids, at least 7 and no more than 12 amino acids or at least 7 and no more than 11 amino acids. In certain embodiments, the linker comprises 15-17 amino acids, and in particular embodiments, comprises 16 amino acids. In some embodiments, the linker comprises 10-20 amino acids. In some embodiments, the linker comprises 14-19 amino acids. In some embodiments, the linker comprises 15-17 amino acids. In some embodiments, the linker comprises 15-16 amino acids. In some embodiments, the linker comprises 16 amino acids. In some embodiments, the linker comprises 3, 4, 5, 6, 7, 8, 9, 10, 11, 12, 13, 14, 15, 16, 17, 18, 19 or 20 amino acids.

In some embodiments, a spacer domain is used. In some embodiments, the spacer domain is derived from CD4, CD8a, CD8b, CD28, CD28T, 4-1BB, or other molecule described herein. In some embodiments, the spacer domains may include a chemically induced dimerizer to control expression upon addition of a small molecule. In some embodiments, a spacer is not used.

The intracellular (signaling) domain of the engineered T cells of the invention may provide signaling to an activating domain, which then activates at least one of the normal effector functions of the immune cell. Effector function of a T cell, for example, may be cytolytic activity or helper activity including the secretion of cytokines.

In certain embodiments, suitable intracellular signaling domain include (i.e., comprise), but are not limited to 4-1BB/CD137, activating NK cell receptors, an Immunoglobulin protein, B7-H3, BAFFR, BLAME (SLAMF8), BTLA, CD100 (SEMA4D), CD103, CD160 (BY55), CD18, CD19, CD19a, CD2, CD247, CD27, CD276 (B7-H3), CD28, CD29, CD3 delta, CD3 epsilon, CD3 gamma, CD30, CD4, CD40, CD49a, CD49D, CD49f, CD69, CD7, CD84, CD8, CD8alpha, CD8beta, CD96 (Tactile), CDl la, CDl lb, CDl lc, CDl ld, CDS, CEACAM1, CRT AM, cytokine receptor, DAP-10, DNAM1 (CD226), Fc gamma receptor, GADS, GITR, HVEM (LIGHTR), IA4, ICAM-1, ICAM-1, Ig alpha (CD79a), IL-2R beta, IL-2R gamma, IL-7R alpha, inducible T cell costimulator (ICOS), integrins, ITGA4, ITGA4, ITGA6, ITGAD, ITGAE, ITGAL, ITGAM, ITGAX, ITGB2, ITGB7, ITGB1, KIRDS2, LAT, LFA-1, LFA-1, ligand that specifically binds with CD83, LIGHT, LIGHT, LTBR, Ly9 (CD229), Lyl08), lymphocyte function-associated antigen-1 (LFA-1; CDl-la/CD18), MHC class 1 molecule, NKG2C, NKG2D, NKp30, NKp44, NKp46, NKp80 (KLRF1), OX-40, PAG/Cbp, programmed death-1 (PD-1), PSGL1, SELPLG (CD162), Signaling Lymphocytic Activation Molecules (SLAM proteins), SLAM (SLAMF1; CD150; IPO-3), SLAMF4 (CD244; 2B4), SLAMF6 (NTB-A, SLAMF7, SLP-76, TNF receptor proteins, TNFR2, TNFSF14, a Toll ligand receptor, TRANCE/RANKL, VLA1, or VLA-6, or a fragment, truncation, or a combination thereof.

A TCR may be introduced to convey antigen reactivity. In some embodiments, the antigen reactivity is restricted by MHC presentation of a peptide. The TCR may be an alpha/beta TCR, gamma/delta TCR, or other. In some embodiments, the TCR is an HPV-16 E7 TCR with murine constant chains (2A linked). In some embodiments, the chains may be linked by an IRES or any 2A family members' sequence (e.g., P2A, T2A, E2A, F2A, etc). In some embodiments, the TCR is an HPV recognizing TCR, or other viral reactive TCR (e.g., EBV, influenza, etc.). In some embodiments, a cancer or cancer associated antigen reactive TCR may be used (e.g., NYESO, MART1, gp100, etc.)

In some embodiments, the TCR is a TCR of normal/healthy peptide reactivity or other antigen reactivity/restriction. In some embodiments, the TCR is reactive against murine or other non-human MHC. In some embodiments, the TCR is a class I or class II restricted TCR.

### Antigen Binding Molecules

Suitable CARs may be engineered to bind to an antigen (such as a cell-surface antigen) by incorporating an antigen binding molecule that interacts with that targeted antigen. In some embodiments, the antigen binding molecule is an antibody fragment thereof, e.g., one or more single chain antibody fragment (“scFv”). A scFv is a single chain antibody fragment having the variable regions of the heavy and light chains of an antibody linked together. See U.S. Pat. Nos. 7,741,465 and 6,319,494, as well as Eshhar et al., Cancer Immunol. Immunotherapy (1997) 45: 131-136. A scFv retains the parent antibody's ability to interact specifically with target antigen. scFv's are useful in chimeric antigen receptors because they may be engineered to be expressed as part of a single chain along with the other CAR components. Id. See also Krause et al., J. Exp. Med., Volume 188, No. 4, 1998 (619-626); Finney et al., Journal of Immunology, 1998, 161: 2791-2797. It will be appreciated that the antigen binding molecule is typically contained within the extracellular portion of the CAR such that it is capable of recognizing and binding to the antigen of interest. Bispecific and multispecific CARs are contemplated within the scope of the invention, with specificity to more than one target of interest.

In some embodiments, the polynucleotide encodes a CAR or a TCR comprising a THD of the present invention and an antigen binding molecule that specifically binds to a target antigen. In some embodiments, the target antigen is a tumor antigen. In some embodiments, the antigen is selected from a tumor-associated surface antigen, such as 5T4, alphafetoprotein (AFP), B7-1 (CD80), B7-2 (CD86), BCMA, B-human chorionic gonadotropin, CA-125, carcinoembryonic antigen (CEA), carcinoembryonic antigen (CEA), CD123, CD133, CD138, CD19, CD20, CD22, CD23, CD24, CD25, CD30, CD33, CD34, CD4, CD40, CD44, CD56, CD8, CLL-1, c-Met, CMV-specific antigen, CS-1, CSPG4, CTLA-4, DLL3, disialoganglioside GD2, ductal-epithelial mucine, EBV-specific antigen, EGFR variant III (EGFRvIII), ELF2M, endoglin, ephrin B2, epidermal growth factor receptor (EGFR), epithelial cell adhesion molecule (EpCAM), epithelial tumor antigen, ErbB2 (HER2/neu), fibroblast associated protein (fap), FLT3, folate binding protein, GD2, GD3, glioma-associated antigen, glycosphingolipids, gp36, HBV-specific antigen, HCV-specific antigen, HER1-HER2, HER2-HER3 in combination, HERV-K, high molecular weight-melanoma associated antigen (HMW-MAA), HIV-1 envelope glycoprotein gp41, HPV-specific antigen, human telomerase reverse transcriptase, IGFI receptor, IGF-II, IL-11Ralpha, IL-13R-a2, Influenza Virus-specific antigen; CD38, insulin growth factor (IGFl)-l, intestinal carboxyl esterase, kappa chain, LAGA-la, lambda chain, Lassa Virus-specific antigen, lectin-reactive AFP, lineage-specific or tissue specific antigen such as CD3, MAGE, MAGE-A1, major histocompatibility complex (MHC) molecule, major histocompatibility complex (MHC) molecule presenting a tumor-specific peptide epitope, M-CSF, melanoma-associated antigen, mesothelin, mesothelin, MN-CA IX, MUC-1, mut hsp70-2, mutated p53, mutated p53, mutated ras, neutrophil elastase, NKG2D, Nkp30, NY-ESO-1, p53, PAP, prostase, prostate specific antigen (PSA), prostate-carcinoma tumor antigen-1 (PCTA-1), prostate-specific antigen protein, STEAP1, STEAP2, PSMA, RAGE-1, ROR1, RU1, RU2 (AS), surface adhesion molecule, surviving and telomerase, TAG-72, the extra domain A (EDA) and extra domain B (EDB) of fibronectin and the Al domain of tenascin-C (TnC Al), thyroglobulin, tumor stromal antigens, vascular endothelial growth factor receptor-2 (VEGFR2), virus-specific surface antigen such as an HIV-specific antigen (such as HIV gpl20), as well as any derivate or variant of these surface markers.

### Vectors, Cells, and Pharmaceutical Compositions

Provided herein is a method of generating modified pluripotent stem cells. Various vectors may be used to introduce a CAR, a TCR a proxy TCR, a pTa protein, or any other exogenous proteins of interest.

Any vector known in the art may be suitable for the present invention. In some embodiments, the vector is a viral vector. In some embodiments, the vector is a retroviral vector, a DNA vector, a murine leukemia virus vector, an SFG vector, a plasmid, a RNA vector, an adenoviral vector, a baculoviral vector, an Epstein Barr viral vector, a papovaviral vector, a vaccinia viral vector, a herpes simplex viral vector, an adenovirus associated vector (AAV), a lentiviral vector, or any combination thereof.

Exogenous promoters may be the human, murine, or any other species sequence of Ubiquitin C, EF1a, PGK, beta-actin, etc. Promoters may use genomic in-frame versions of these sequences, fractions such as spliced out introns, introns intact, or any fractional junction of these sequences. Promoters may also be derived from viral elements, such as LTRs. Viruses of origin for promoters may be MPSV, MSGV, HTLV, HIV, etc. Spacer domains may include a throttle/chemically induced dimerizer to control expression upon addition of a small molecule in a titratable fashion.

The cell of the present invention may be obtained through any source known in the art. For example, T cells may be differentiated in vitro from a hematopoietic stem cell population, or T cells may be obtained from a subject. T cells may be obtained from, e.g., peripheral blood mononuclear cells, bone marrow, lymph node tissue, cord blood, thymus tissue, tissue from a site of infection, ascites, pleural effusion, spleen tissue, and tumors. In addition, the T cells may be derived from one or more T cell lines available in the art. T cells may also be obtained from a unit of blood collected from a subject using any number of techniques known to the skilled artisan, such as FICOLL™ separation and/or apheresis. In certain embodiments, the cells collected by apheresis are washed to remove the plasma fraction, and placed in an appropriate buffer or media for subsequent processing. In some embodiments, the cells are washed with PBS. As will be appreciated, a washing step may be used, such as by using a semiautomated flow-through centrifuge, e.g., the Cobe™ 2991 cell processor, the Baxter CytoMate™, or the like. In some embodiments, the washed cells are resuspended in one or more biocompatible buffers, or other saline solution with or without buffer. In certain embodiments, the undesired components of the apheresis sample are removed. Additional methods of isolating T cells for a T cell therapy are disclosed in U.S. Patent Publication No. 2013/0287748, which is herein incorporated by references in its entirety.

In certain embodiments, stem cells are isolated from PBMCs by lysing the red blood cells and depleting the monocytes, e.g., by using centrifugation through a PERCOLL™ gradient. In some embodiments, a specific subpopulation of T cells, such as CD4⁺, CD8⁺, CD28⁺, CD45RA⁺, and CD45RO⁺ T cells is further isolated by positive or negative selection techniques known in the art. For example, enrichment of a T cell population by negative selection may be accomplished with a combination of antibodies directed to surface markers unique to the negatively selected cells. In some embodiments, cell sorting and/or selection via negative magnetic immunoadherence or flow cytometry that uses a cocktail of monoclonal antibodies directed to cell surface markers present on the cells negatively selected may be used. For example, to enrich for CD4⁺ cells by negative selection, a monoclonal antibody cocktail typically includes antibodies to CD8, CD11b, CD14, CD16, CD20, and HLA-DR. In certain embodiments, flow cytometry and cell sorting are used to isolate cell populations of interest for use in the present invention.

In some embodiments, PBMCs are used directly for genetic modification with the immune cells (such as CARs or TCRs) using methods as described herein. In certain embodiments, after isolating the PBMCs, T lymphocytes are further isolated, and both cytotoxic and helper T lymphocytes are sorted into naive, stem cell memory, central memory, effector memory, and effector T cell subpopulations either before or after genetic modification and/or expansion.

In some embodiments, CD8⁺ cells are further sorted into naive, stem cell memory, central memory, effector memory, and effector cells by identifying cell surface antigens that are associated with each of these types of CD8⁺ cells. In some embodiments, phenotypic markers of central memory T cells include CCR7, CD3, CD28, CD45RO, CD62L, and CD127 and are negative for granzyme B. In some embodiments, central memory T cells are CD8⁺, CD45RO⁺, and CD62L⁺ T cells. In some embodiments, effector T cells are negative for CCR7, CD28, CD62L, and CD127 and positive for granzyme B and perforin. In certain embodiments, CD4⁺ T cells are further sorted into subpopulations. For example, CD4⁺ T helper cells may be sorted into naive, central memory and effector cells by identifying cell populations that have cell surface antigens.

In some embodiments, the immune cells, e.g., T cells, are genetically modified following isolation using known methods, or the immune cells are activated and expanded (or differentiated in the case of progenitors) in vitro prior to being genetically modified. In another embodiment, the immune cells, e.g., T cells, are genetically modified with the chimeric antigen receptors described herein (e.g., transduced with a viral vector comprising one or more nucleotide sequences encoding a CAR) and then are activated and/or expanded in vitro. Methods for activating and expanding T cells are known in the art and are described, e.g., in U.S. Pat. Nos. 6,905,874; 6,867,041; and 6,797,514, and PCT Publication No. WO 2012/079000, the contents of which are hereby incorporated by reference in their entirety. Generally, such methods include contacting PBMC or isolated T cells with a stimulatory agent and costimulatory agent, such as anti-CD3 and anti-CD28 antibodies, generally attached to a bead or other surface, in a culture medium with appropriate cytokines, such as IL-2. Anti-CD3 and anti-CD28 antibodies attached to the same bead serve as a “surrogate” antigen presenting cell (APC). One example is The Dynabeads® system, a CD3/CD28 activator/stimulator system for physiological activation of human T cells. In other embodiments, the T cells are activated and stimulated to proliferate with feeder cells and appropriate antibodies and cytokines using methods such as those described in U.S. Pat. Nos. 6,040,177 and 5,827,642, and PCT Publication No. WO 2012/129514, the contents of which are hereby incorporated by reference in their entirety.

In certain embodiments, the T cells are obtained from a donor subject. In some embodiments, the donor subject is human patient afflicted with a cancer or a tumor. In other embodiments, the donor subject is a human patient not afflicted with a cancer or a tumor.

Other aspects of the present invention are directed to compositions comprising a polynucleotide described herein, a vector described herein, a polypeptide described herein, or an in vitro cell described herein. In some embodiments, the composition comprises a pharmaceutically acceptable carrier, diluent, solubilizer, emulsifier, preservative, and/or adjuvant. In some embodiments, the composition comprises an excipient. In one embodiment, the composition comprises a polynucleotide encoding a CAR or a TCR comprising a truncated hinge domain (“THD”) described herein. In another embodiment, the composition comprises a CAR or a TCR comprising a TCD encoded by a polynucleotide of the present invention. In another embodiment, the composition comprises a T cell comprising a CAR or a TCR comprising a TCD described herein.

In other embodiments, the composition is selected for parenteral delivery, for inhalation, or for delivery through the digestive tract, such as orally. The preparation of such pharmaceutically acceptable compositions is within the ability of one skilled in the art. In certain embodiments, buffers are used to maintain the composition at physiological pH or at a slightly lower pH, typically within a pH range of from about 5 to about 8. In certain embodiments, when parenteral administration is contemplated, the composition is in the form of a pyrogen-free, parenterally acceptable aqueous solution comprising a composition described herein, with or without additional therapeutic agents, in a pharmaceutically acceptable vehicle. In certain embodiments, the vehicle for parenteral injection is sterile distilled water in which composition described herein, with or without at least one additional therapeutic agent, is formulated as a sterile, isotonic solution, properly preserved. In certain embodiments, the preparation involves the formulation of the desired molecule with polymeric compounds (such as polylactic acid or polyglycolic acid), beads or liposomes, that provide for the controlled or sustained release of the product, which are then be delivered via a depot injection. In certain embodiments, implantable drug delivery devices are used to introduce the desired molecule.

### Cell Differentiation

The modified pluripotent cell product may be differentiated into a T cell using the artificial thymic organoid (ATO) system, notch agonist, OP9-DLL1, OP9-DLL4, fetal thymic organoid culture (FTOC), chemical induction, bone marrow/liver/thymus or other humanized mouse, embryoid body (EB), or other differentiation technology.

The differentiated cell type may be a CD8 single positive T cell, a CD4 single positive T cell, a CD4 CD8 double positive T cell, a double negative T cell, a CD3 positive cell, an NK cell, a proT cell, a pre-proT cell, a mesodermal progenitor, a B cell, a common lymphoid progenitor, a hematopoietic progenitor, a hematopoietic stem cell, etc.

### Artificial Thymic Organoid (ATO)

In vivo genetically modified murine models, humanized mice, and in vitro systems such as the OP9-DLL1 or recently described artificial thymic organoid (ATO) have shown multiple avenues by which stem cells may be modified or cultured to generate a desired mature T cell, including with antigen receptors against cancer antigens.

Modified pluripotent stem cells according to the present invention may be further differentiated in the OP9-DLL1 or Artificial Thymic Organoid (ATO) cell culture system. An ATO is a serum-free, 3-dimensional cell culture technology that recapitulates T-cell differentiation. ATO technology has the potential to generate off-the-shelf engineered T cells to treat cancer and other diseases.

A suitable artificial thymic organoid (ATO) system supports highly efficient in vitro differentiation and positive selection of native and TCR-engineered human T cells from cord blood, bone marrow, and peripheral blood HSPCs. ATO-derived T cells exhibit a naïve phenotype, diverse TCR repertoire, and TCR-dependent activation and proliferation. ATO-derived engineered T cells also mature to a naïve phenotype and furthermore show antigen specific tumor killing in vitro and in vivo. ATOs thus present an efficient method for the generation of naïve and potentially non-alloreactive engineered T cells for adoptive cell therapy. Exemplary methods for producing engineered T cells with the ATO culture system are described in, for example, U.S. Provisional Patent Application Nos. 62/511,907, 62/514,467, Evseenko et al., 2010 PNAS, Seet et al., 2017 Nature Methods, the contents of which are incorporated herein by reference. Other exemplary methods relating to the ATO culture system are described in International Patent Publications No WO2017/075389.

TCR engineered stem cells generate T cells in the ATO system. Additionally, ATO derived T cells exhibit TCR diversity and allelic exclusion. Engineered stem cells in the ATO system exhibit a markedly restricted TCR by Vbeta antibody panel flow cytometric investigation providing evidence of allelic exclusion

### Methods of Producing a Desired T-cell Lineage

In some embodiments, modified pluripotent cells are further engineered for genome editing of critical developmental genes to eliminate cell impurities and modulate activity of T cell differentiation products from the ATO platform.

During the process of differentiating a stem cell into an immune cell, undesired cellular lineage by-products may potentially arise. For example, in the case of differentiating a stem cell to an alpha-beta T-cell, NK cells, regulatory T-cells (Tregs), gamma-delta T-cells, and other non-immune cell types may also develop in the culture. The methods described herein, utilizes any genome editing platform (CRISPR/Cas9, TALENs, megaTALs, meganucleases, Cpf1, ZFN, etc) to knock-out or modify certain critical master cell fate regulators, such as transcription factors, with to impair or eliminate the generation of undesired cell by-products.

### Cancer Treatment

The methods described herein may be used to treat a cancer in a subject, reduce the size of a tumor, kill tumor cells, prevent tumor cell proliferation, prevent growth of a tumor, eliminate a tumor from a patient, prevent relapse of a tumor, prevent tumor metastasis, induce remission in a patient, or any combination thereof. In certain embodiments, the methods induce a complete response. In other embodiments, the methods induce a partial response. In some embodiments, the treatment is intended for adult and/or pediatric patients.

In some embodiments, the cell product may be used in oncology, immunosuppression, autoimmune control, vaccine or as a prophylactic measure. The cell may be used as a commercial product, a clinical trial, preclinical work, basic research. The cell may be used for human and/or veterinary medicine. In some embodiments, the cell product may be used as a detection reagent/discovery research.

Cancers that may be treated include tumors that are not vascularized, not yet substantially vascularized, or vascularized. The cancer may also include solid or non-solid tumors. In some embodiments, the cancer is a hematologic cancer. In some embodiments, the cancer is of the white blood cells. In other embodiments, the cancer is of the plasma cells. In some embodiments, the cancer is leukemia, lymphoma, or myeloma. In certain embodiments, the cancer is acute lymphoblastic leukemia (ALL) (including non T cell ALL), acute lymphoid leukemia (ALL), and hemophagocytic lymphohistocytosis (HLH)), B cell prolymphocytic leukemia, B-cell acute lymphoid leukemia (“BALL”), blastic plasmacytoid dendritic cell neoplasm, Burkitt's lymphoma, chronic lymphocytic leukemia (CLL), chronic myelogenous leukemia (CML), chronic myeloid leukemia (CML), chronic or acute granulomatous disease, chronic or acute leukemia, large B cell lymphoma, diffuse large B cell lymphoma (DLBCL), follicular lymphoma, follicular lymphoma (FL), hairy cell leukemia, hemophagocytic syndrome (Macrophage Activating Syndrome (MAS), Hodgkin's Disease, large cell granuloma, leukocyte adhesion deficiency, malignant lymphoproliferative conditions, MALT lymphoma, mantle cell lymphoma, Marginal zone lymphoma, monoclonal gammapathy of undetermined significance (MGUS), multiple myeloma, myelodysplasia and myelodysplastic syndrome (MDS), myeloid diseases including but not limited to acute myeloid leukemia (AML), non-Hodgkin's lymphoma (NHL), plasma cell proliferative disorders (e.g., asymptomatic myeloma (smoldering multiple myeloma or indolent myeloma), plasmablastic lymphoma, plasmacytoid dendritic cell neoplasm, plasmacytomas (e.g., plasma cell dyscrasia; solitary myeloma; solitary plasmacytoma; extramedullary plasmacytoma; and multiple plasmacytoma), POEMS syndrome (Crow-Fukase syndrome; Takatsuki disease; PEP syndrome), primary mediastinal large B cell lymphoma (PMBC), small cell- or a large cell-follicular lymphoma, splenic marginal zone lymphoma (SMZL), systemic amyloid light chain amyloidosis, T-cell acute lymphoid leukemia (“TALL”) T-cell lymphoma, transformed follicular lymphoma, Waldenstrom macroglobulinemia, or a combination thereof.

In some embodiments, the cancer is a myeloma. In one particular embodiment, the cancer is multiple myeloma. In some embodiments, the cancer is leukemia. In some embodiments, the cancer is acute myeloid leukemia.

In some embodiments, the cancer is relapsed or refractory large B-cell lymphoma, diffuse large B-cell lymphoma (DLBCL) not otherwise specified, primary mediastinal large B-cell lymphoma, high grade B-cell lymphoma, or DLBCL arising from follicular lymphoma.

In some embodiments, the methods further comprise administering a chemotherapeutic. In certain embodiments, the chemotherapeutic selected is a lymphodepleting (preconditioning) chemotherapeutic. Beneficial preconditioning treatment regimens, along with correlative beneficial biomarkers, are described in U.S. Provisional Patent Applications, 62/262,143 and 62/167,750, which are hereby incorporated by reference, in their entirety herein. These describe, e.g., methods of conditioning a patient in need of a T cell therapy comprising administering to the patient specified beneficial doses of cyclophosphamide (between 200 mg/m²/day and 2000 mg/m²/day) and specified doses of fludarabine (between 20 mg/m²/day and 900 mg/m²/day). One such dose regimen involves treating a patient comprising administering daily to the patient about 500 mg/m²/day of cyclophosphamide and about 60 mg/m²/day of fludarabine for three days prior to administration of a therapeutically effective amount of engineered. T cells to the patient.

In other embodiments, the antigen binding molecule, transduced (or otherwise engineered) cells (such as CARs or TCRs), and the chemotherapeutic agent are administered each in an amount effective to treat the disease or condition in the subject.

In certain embodiments, compositions comprising CAR- and/or TCR-expressing immune effector cells disclosed herein may be administered in conjunction with any number of chemotherapeutic agents. Examples of chemotherapeutic agents include alkylating agents such as thiotepa and cyclophosphamide (CYTOXAN™); alkyl sultanates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethylenethiophosphaoramide and trimethylolomelamine resume; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as aclacinomysins, actinomycin, authramycin, azaserine, bleomycins, cactinomycin, calicheamicin, carabicin, carminomycin, carzinophilin, chromomycins, dactinomycin, daunorubicin, detorubicin, 6-diazo-5-oxo-L-norleucine, doxorubicin, epirubicin, esorubicin, idarubicin, marcellomycin, mitomycins, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine, 5-FU; androgens such as calusterone, dromostanolone propionate, epitiostanol, mepitiostane, testolactone; anti-adrenals such as aminoglutethimide, mitotane, trilostane; folic acid replenishes such as frolinic acid; aceglatone; aldophosphamide glycoside; aminolevulinic acid; amsacrine; bestrabucil; bisantrene; edatraxate; defofamine; demecolcine; diaziquone; elformithine; elliptinium acetate; etoglucid; gallium nitrate; hydroxyurea; lentinan; lonidamine; mitoguazone; mitoxantrone; mopidamol; nitracrine; pentostatin; phenamet; pirarubicin; podophyllinic acid; 2-ethylhydrazide; procarbazine; PSK®; razoxane; sizofiran; spirogermanium; tenuazonic acid; triaziquone; 2,2′,2″-trichlorotriethylamine; urethan; vindesine; dacarbazine; mannomustine; mitobronitol; mitolactol; pipobroman; gacytosine; arabinoside (“Ara-C”); cyclophosphamide; thiotepa; taxoids, e.g. paclitaxel (TAXOL™, Bristol-Myers Squibb) and doxetaxel (TAXOTERE™, Rhone-Poulenc Rorer); chlorambucil; gemcitabine; 6-thioguanine; mercaptopurine; methotrexate; platinum analogs such as cisplatin and carboplatin; vinblastine; platinum; etoposide (VP-16); ifosfamide; mitomycin C; mitoxantrone; vincristine; vinorelbine; navelbine; novantrone; teniposide; daunomycin; aminopterin; xeloda; ibandronate; CPT-11; topoisomerase inhibitor RFS2000; difluoromethylomithine (DMFO); retinoic acid derivatives such as Targretin™(bexarotene), Panretin™, (alitretinoin); ONTAK™(denileukin diftitox); esperamicins; capecitabine; and pharmaceutically acceptable salts, acids or derivatives of any of the above. In some embodiments, compositions comprising CAR- and/or TCR-expressing immune effector cells disclosed herein may be administered in conjunction with an anti-hormonal agent that acts to regulate or inhibit hormone action on tumors such as anti-estrogens including for example tamoxifen, raloxifene, aromatase inhibiting 4(5)-imidazoles, 4-hydroxytamoxifen, trioxifene, keoxifene, LY117018, onapristone, and toremifene (Fareston); and anti-androgens such as flutamide, nilutamide, bicalutamide, leuprolide, and goserelin; and pharmaceutically acceptable salts, acids or derivatives of any of the above. Combinations of chemotherapeutic agents are also administered where appropriate, including, but not limited to CHOP, i.e., Cyclophosphamide (Cytoxan®), Doxorubicin (hydroxydoxorubicin), Vincristine (Oncovin®), and Prednisone.

In some embodiments, the chemotherapeutic agent is administered at the same time or within one week after the administration of the engineered cell or nucleic acid. In other embodiments, the chemotherapeutic agent is administered from 1 to 4 weeks or from 1 week to 1 month, 1 week to 2 months, 1 week to 3 months, 1 week to 6 months, 1 week to 9 months, or 1 week to 12 months after the administration of the engineered cell or nucleic acid. In some embodiments, the chemotherapeutic agent is administered at least 1 month before administering the cell or nucleic acid. In some embodiments, the methods further comprise administering two or more chemotherapeutic agents.

A variety of additional therapeutic agents may be used in conjunction with the compositions described herein. For example, potentially useful additional therapeutic agents include PD-1 inhibitors such as nivolumab (OPDIVO®), pembrolizumab (KEYTRUDA®), pembrolizumab, pidilizumab (CureTech), and atezolizumab (Roche). Other potential useful additional therapeutic agents include 4-1BB (may also be referred to as CD137/TNFRSF9) inhibitors such as urelumab and utomilumab.

Additional therapeutic agents suitable for use in combination with the invention include, but are not limited to, ibrutinib (IMBRUVICVA®), ofatumumab (ARZERRA®), rituximab (RITUXAN®), bevacizumab (AVASTIN®), trastuzumah (HERCEPTIN®), trastuzumab emtansine (KADCYLA®), imatinib (GLEEVEC®), cetuximab (ERBITUX®), panitumumab (VECTIBIX®), catumaxomab, ibritumomab, ofatumumab, tositumomab, brentuximab, alemtuzumab, gemtuzumab, erlotinib, gefitinib, vandetanib, afatinib, lapatinib, neratinib, axitinib, masitinib, pazopanib, sunitinib, sorafenib, toceranib, lestaurtinib, axitinib, cediranib, lenvatinib, nintedanib, pazopanib, regorafenib, semaxanib, sorafenib, sunitinib, tivozanib, toceranib, vandetanib, entrectinib, cabozantinib, imatinib, dasatinib, nilotinib, ponatinib, radotinib, bosutinib, lestaurtinib, ruxolitinib, pacritinib, cobimetinib, selumetinib, trametinib, binimetinib, alectinib, ceritinib, crizotinib, aflibercept, adipotide, denileukin diftitox, mTOR inhibitors such as Everolimus and Temsirolimus, hedgehog inhibitors such as sonidegib and vismodegib, CDK inhibitors such as CDK inhibitor (palbociclib).

In additional embodiments, the composition comprising CAR- and/or TCR-containing immune are administered with an anti-inflammatory agent. Anti-inflammatory agents or drugs may include, but are not limited to, steroids and glucocorticoids (including betamethasone, budesonide, dexamethasone, hydrocortisone acetate, hydrocortisone, hydrocortisone, methylprednisolone, prednisolone, prednisone, and triamcinolone), nonsteroidal anti-inflammatory drugs (NSAIDS) including aspirin, ibuprofen, naproxen, methotrexate, sulfasalazine, leflunomide, anti-TNF medications, cyclophosphamide, and mycophenolate. Exemplary NSAIDs include ibuprofen, naproxen, naproxen sodium, Cox-2 inhibitors, and sialylates. Exemplary analgesics include acetaminophen, oxycodone, and tramadol of proporxyphene hydrochloride. Exemplary glucocorticoids include cortisone, dexamethasone, hydrocortisone, methylprednisolone, prednisolone, or prednisone. Exemplary biological response modifiers include molecules directed against cell surface markers (e.g., CD4, CD5, etc.), cytokine inhibitors, such as the TNF antagonists, (e.g., etanercept (ENBREL®), adalimumab (HUMIRA®) and infliximab (REMICADE®), chemokine inhibitors and adhesion molecule inhibitors. The biological response modifiers include monoclonal antibodies as well as recombinant forms of molecules. Exemplary DMARDs include azathioprine, cyclophosphamide, cyclosporine, methotrexate, penicillamine, leflunomide, sulfasalazine, hydroxychloroquine, Gold (oral (auranofin) and intramuscular), and minocycline.

In certain embodiments, the compositions described herein are administered in conjunction with a cytokine. “Cytokine” as used herein is meant to refer to proteins released by one cell population that act on another cell as intercellular mediators. Examples of cytokines are lymphokines, monokines, and traditional polypeptide hormones. Included among the cytokines are growth hormones such as human growth hormone, N-methionyl human growth hormone, and bovine growth hormone; parathyroid hormone; thyroxine; insulin; proinsulin; relaxin; prorelaxin; glycoprotein hormones such as follicle stimulating hormone (FSH), thyroid stimulating hormone (TSH), and luteinizing hormone (LH); hepatic growth factor (HGF); fibroblast growth factor (FGF); prolactin; placental lactogen; mullerian-inhibiting substance; mouse gonadotropin-associated peptide; inhibin; activin; vascular endothelial growth factor; integrin; thrombopoietin (TPO); nerve growth factors (NGFs) such as NGF-beta; platelet-growth factor; transforming growth factors (TGFs) such as TGF-alpha and TGF-beta; insulin-like growth factor-I and -II; erythropoietin (EPO); osteoinductive factors; interferons such as interferon-alpha, beta, and -gamma; colony stimulating factors (CSFs) such as macrophage-CSF (M-CSF); granulocyte-macrophage-CSF (GM-CSF); and granulocyte-CSF (G-CSF); interleukins (ILs) such as IL-1, IL-1alpha, IL-2, IL-3, IL-4, IL-5, IL-6, IL-7, IL-8, IL-9, IL-10, IL-11, IL-12; IL-15, a tumor necrosis factor such as TNF-alpha or TNF-beta; and other polypeptide factors including LIF and kit ligand (KL). As used herein, the term cytokine includes proteins from natural sources or from recombinant cell culture, and biologically active equivalents of the native sequence cytokines.

Another aspect of the present invention is directed to a method of inducing immunity against a tumor comprising administering to a subject an effective amount of a modified T cell disclosed herein. Another aspect of the present invention is directed to a method of inducing an immune response in a subject comprising administering an effective amount of the engineered immune cells of the present application. In some embodiments, the immune response is a T cell-mediated immune response. In some embodiments, the T cell-mediated immune response is directed against one or more target cells. In some embodiments, the engineered immune cell comprises a CAR or a TCR, wherein the CAR or the TCR comprises a THD described in the present disclosure. In some embodiments, the target cell is a tumor cell.

Another aspect of the present invention is directed to a method for treating or preventing a malignancy, said method comprising administering to a subject in need thereof an effective amount of at least one immune cell, wherein the immune cell comprises at least one CAR or TCR.

Another aspect of the present invention is directed to a method of treating a cancer in a subject in need thereof comprising administering to the subject a polynucleotide, a vector, a CAR or a TCR, a cell, or a composition disclosed herein. In one embodiment, the method comprises administering a polynucleotide encoding a CAR or a TCR. In another embodiment, the method comprises administering a vector comprising a polynucleotide encoding a CAR or a TCR. In another embodiment, the method comprises administering a CAR or a TCR encoded by a polynucleotide disclosed herein. In another embodiment, the method comprises administering a cell comprising the polynucleotide, or a vector comprising the polynucleotide, encoding a CAR or a TCR.

In some embodiments, the donor T cells for use in the T cell therapy are obtained from the patient (e.g., for an autologous T cell therapy). In other embodiments, the donor stem cells to be differentiated into T cells for use in the T cell therapy are obtained from a subject that is not the patient.

The T cells may be administered at a therapeutically effective amount. For example, a therapeutically effective amount of the T cells may be at least about 10⁴ cells, at least about 10⁵ cells, at least about 10⁶ cells, at least about 10⁷ cells, at least about 10⁸ cells, at least about 10⁹, or at least about 10¹⁰. In another embodiment, the therapeutically effective amount of the T cells is about 10⁴ cells, about 10⁵ cells, about 10⁶ cells, about 10⁷ cells, or about 10⁸ cells. In one particular embodiment, the therapeutically effective amount of the CAR T cells or the TCR T cells is about 2×10⁶ cells/kg, about 3×10⁶ cells/kg, about 4×10⁶ cells/kg, about 5×10⁶ cells/kg, about 6×10⁶ cells/kg, about 7×10⁶ cells/kg, about 8×10⁶ cells/kg, about 9×10⁶ cells/kg, about 1×10⁷ cells/kg, about 2×10⁷ cells/kg, about 3×10⁷ cells/kg, about 4×10⁷ cells/kg, about 5×10⁷ cells/kg, about 6×10⁷ cells/kg, about 7×10⁷ cells/kg, about 8×10⁷ cells/kg, or about 9×10⁷ cells/kg. In another embodiment, the therapeutically effective amount of the CAR T cells or the TCR T cells is about 1×10⁵ cells/kg, about 2×10⁵ cells/kg, about 3×10⁵ cells/kg, about 4×10⁵ cells/kg, about 5×10⁵ cells/kg, about 6×10⁵ cells/kg, about 7×10⁵ cells/kg, about 8×10⁵ cells/kg, or about 9×10⁵ cells/kg.

### Immune Tolerance

The methods of the invention may be used to treat an immune tolerance disease in a subject. In certain embodiments, the methods induce a complete response. In other embodiments, the methods induce a partial response.

Deficits in central or peripheral tolerance may cause autoimmune disease, resulting in syndromes such as systemic lupus erythematosus, rheumatoid arthritis, type 1 diabetes, autoimmune polyendocrine syndrome type 1 (APS-1), and immunodysregulation polyendocrinopathy enteropathy X-linked syndrome (IPEX), and potentially contribute to asthma, allergy, and inflammatory bowel disease. Immune tolerance may also be problematic in transplantation rejection for example stem cell transplant, kidney transplant, liver transplant, etc.

HSC, ES or iPS cells engineered to eliminate endogenous TCR or HLA expression may be further engineered to express specific CARs, TCRs, or other antigen recognition molecules according to the therapeutic target.

All publications, patents, and patent applications mentioned in this specification are herein incorporated by reference to the same extent as if each individual publication, patent, or patent application was specifically and individually indicated to be incorporated by reference. However, the citation of a reference herein should not be construed as an acknowledgement that such reference is prior art to the present invention. To the extent that any of the definitions or terms provided in the references incorporated by reference differ from the terms and discussion provided herein, the present terms and definitions control.

The present invention is further illustrated by the following examples, which should not be construed as further limiting. The contents of all references cited throughout this application are expressly incorporated herein by reference.

### EXAMPLES

### Example 1: Generation of Modified Pluripotent Stem Cells

This example illustrates characterization of PBMCs and purified T cells for reprogramming to iPSCs and preparation of modified pluripotent stem cells engineered to eliminate endogenous TCR or HLA expression.

PBMCs were isolated from three apheresis units using Ficoll and T cells were negatively selected (touchless selected) from the same apheresis units using Miltenyi Pan T Cell Isolation kit. The donors (Subject A, B, and C) of the apheresis unit were female, under the age of 25, non-smoker, non-drinker, with no history of genetic diseases of the blood or other tissues. Isolated PBMCs and purified T cells were analyzed by flow cytometry using antibodies against CD56, CD14, CD19, or TCRα/β before cryopreservation. The purity of T cells was characterized by the presence of TCRα/β and the absence of CD14, CD19, and CD56. Results showed the methods isolated and purified T cells (data not shown).

PBMCs and T cells were further analyzed by karyotyping to evaluate chromosomal abnormalities (KaryoStat assay, Thermofisher) before reprogramming. All PBMCs and T cells from the three donors showed normal karyotype (i.e. normal chromosomal arrangement) (data not shown).

These cells were reprogrammed to induced pluripotent stem cells (iPSCs) using Yamanaka factors (Oct3/4, Sox2, Klf4, c-Myc) delivered via a modified Sendai virus (CytoTune 2.0). Ten iPSC clones were isolated and expanded out to clonal iPSCs line and banked for each input cell population. All clones were stained positive for TRA-1-60 by immunofluorescence staining (data not shown).

The pluripotency of each iPSC clonal line was assessed by the Pluripotency Scorecard Assay. The expression levels of a panel of pluripotency and three primary germ layer markers were compared against those from a set of known human PSCs and their differentiated counterparts. A positive value indicates the expression levels of the markers in the sample are comparable or higher than those in the reference. A value greater than 1.5 in the scorecard analysis indicates the markers were upregulated. A negative value indicates the expression levels of the markers in the sample are lower than those in reference. All clonal lines showed positive for pluripotency and negative for three primary germ layers. Representative results of PSC scorecard analysis of PBMC and T cell derived iPSC clones and embryoid bodies (EBs) are shown in Table 2.

TABLE 2
Results of PSC scorecard analysis of PBMC and T cell derived iPSC clones

| Sample Name | Sample Type | Self-renewal | Ectoderm | Mesoderm | Endoderm |
|---|---|---|---|---|---|
| Subject A PBMC Clone 16 | iPSCs | −0.59 | −0.47 | −1.30 | −1.33 |
| Subject B PBMC Clone 16 | | 0.26 | −0.69 | −0.78 | −1.35 |
| Subject C PBMC Clone 3 | | 0.23 | −0.48 | −1.03 | −1.47 |
| Subject A T-Cell Clone 34 | | −0.15 | −0.58 | −0.89 | −1.54 |
| Subject B T-Cell Clone 7 | | −0.56 | −0.34 | −0.86 | −1.66 |
| Subject C T-Cell Clone 42 | | −0.62 | −0.48 | −1.36 | −1.91 |
| Subject A PBMC Clone 16 | EBs | −3.59 | −0.08 | 5.18 | 0.38 |
| Subject B PBMC Clone 16 | | −4.97 | 0.52 | 6.05 | 0.73 |
| Subject C PBMC Clone 3 | | −2.60 | 0.65 | 4.84 | 0.81 |
| Subject A T-Cell Clone 34 | | −6.02 | 0.41 | 5.58 | 1.08 |
| Subject B T-Cell Clone 7 | | −2.96 | 0.13 | 5.26 | 0.59 |
| Subject C T-Cell Clone 42 | | −0.55 | 0.18 | 4.56 | 0.44 |

Gene expression relative to the reference standard: x > 1.5 upregulated; 1.0 < x <= 0.5 higher than reference standard; −0.5 <= x <= 0.5 comparable; −0.5 < x < −1.5 lower than reference standard; x < −1.5 downregulated

The reprogrammed cell is expanded out to a clonal cell line and banked. The cell line is whole genome sequenced to establish identity and the sequence of loci for targeted gene editing, in particular the alpha and beta T cell receptor loci.

The TCRα constant (TRAC) locus is edited using zinc finger nucleases as designed by Sangamo Therapeutics. These ZFN are introduced to the iPSC by electroporation using the Thermo Fisher Neon electroporation system. A construct encoding the FMC63 CD19 CAR with CD28 costimulatory domain and CD3 zeta is delivered to cells using adeno associated virus serotype 6 (AAV6). The construct targets the TRAC locus, taking advantage of the endogenous TRAC promoter to drive CAR expression.

The TCRβ constant (TRBC) locus is edited using zinc finger nucleases as designed by Sangamo Therapeutics. These ZFN are introduced to the iPSC by electroporation using the Thermo Fisher Neon electroporation system. A construct encoding the HPV-16 E7 TCR is delivered to cells using AAV6. The TCR is inserted into the TRBC locus to drive the development of alpha beta (TCRαβ) T cells from iPSC.

The beta 2 microglobulin (b2m) locus is edited using zinc finger nucleases as designed by Sangamo Therapeutics. These ZFN are introduced to the iPSC by electroporation using the Thermo Fisher Neon electroporation system. A construct encoding an HLA-E single chain trimer (HLA-E SCT) is delivered to cells using AAV6. The b2m locus is edited to eliminate expression of class 1a HLA molecules and prevent recognition of these cells by T cells. The HLA-E SCT is inserted to the b2m locus to prevent recognition of these cells by natural killer (NK) cells.

The gene edited iPSCs are made into a master cell bank, whole genome sequenced to identify off target cutting or integration. The master cell bank is karyotyped. In subsequent studies, the TCRα constant (TRAC) locus and beta 2 microglobulin (b2m) locus were edited or modified. In the TRAC study, the construct encoding the FMC63 CD19 CAR with CD28 costimulatory domain and CD3 zeta (SEQ ID No: 1) was delivered to 179i and/or 202i human iPSCs using ZFN. The resulting human iPSC pool populations were cultured and characterized before single clone generation by flow cytometry analysis (FACS).

The iPSC pool populations were cultured for 8 days and harvested for genomic DNA extraction. A region of 250 bp flanking the target site from control (no ZFN treatment) and edited pool (ZFN treatment) was amplified by PCR, sequenced, and analyzed by TIDE (tracking of insertion/deletion by decomposition) (Brinkman et al. 2014 Nucl. Acids Res. 42(22): e168). In TIDE analysis, a score >0 indicates insertion and a score <0 indicates deletion. An insertion or deletion in a size that is not a multiple of 3 indicates a frame-shifting and may potentially lead to loss of TRAC protein. The results of TIDE analysis of polyclonal populations are shown in Table 3.

TABLE 3
Results of TIDE analysis in TRAC ZFN treated 202i cells

| indel size | percentage | p-value |
|---|---|---|
| 4 | 9.1 | 2.80E−86 |
| −11 | 5.5 | 1.80E−31 |
| −1 | 4.7 | 5.40E−24 |
| 1 | 3.7 | 2.10E−15 |
| −22 | 2.8 | 2.80E−09 |
| −12 | 1.8 | 9.30E−05 |
| −10 | 1.4 | 0.0027 |
| −7 | 1.3 | 0.0069 |
| −3 | 1.3 | 0.0041 |

Single clones were cultured for 14 days and cells were harvested for genomic DNA extraction. The target allele was amplified and characterized by northern blotting analysis. The results of 202i PSCs and 179i iPSC single clones show the insertion of the CD19 CAR into TRAC locus in several single clones (data not shown). In addition, single clones were characterized by digital droplet PCR (ddPCR) and primer/probe sets specific to the targeted alleles to determine the copy number of insertions. The single clones with 2 copy of CD19 CAR knock-in (CAR-KI-TRAC) allele and 0 copy of wild-type allele were selected further studies. In the b2m study, an enhanced green fluorescent protein (EGFP) was inserted between homologous arms of about 800 bp flanking the targeted cut site. The resulting iPSC 202i pool populations were cultured and characterized by TIDE analysis (Table 4) and flow cytometry analysis of β2-microgloblin and GFP expression (data not shown).

TABLE 4
Results of TIDE analysis in b2m ZFN treated 202i cells

| indel size | percentage | p-value |
|---|---|---|
| −14 | 11.9 | 1.10E−126 |
| −1 | 9.2 | 4.20E−79 |
| 3 | 7 | 2.20E−48 |
| −34 | 5.1 | 2.90E−24 |
| −19 | 5.1 | 1.20E−24 |
| −9 | 4.6 | 6.80E−21 |
| −2 | 4.6 | 1.40E−21 |
| −5 | 4.2 | 7.90E−18 |
| −13 | 3.9 | 7.90E−15 |
| −23 | 3.8 | 5.10E−14 |
| −8 | 3.4 | 5.40E−12 |
| −11 | 2.1 | 1.80E−05 |
| −6 | 2.1 | 3.20E−05 |
| −20 | 2 | 6.50E−05 |
| −17 | 1.6 | 0.00093 |
| −42 | 1.3 | 0.0075 |

### Example 2: T Cell Differentiation From Modified Pluripotent Stem Cells

iPSCs are induced to differentiate to mesoderm progenitors (hEMP). hEMPs are complexed with MS5 cells transduced to express hDLL4. 1×10⁴ hEMP are combined with 5×10⁵ MS5. Cells are centrifuged, supernatant is removed, and cells are deposited as a droplet onto a 0.4 um PTFE membrane.

ATOs are grown for 6 weeks. ATOs are harvested from membranes, deposited into Miltenyi gentleMACS C tubes, and run on the Miltenyi gentleMACS dissociator using program EB01. Cell suspensions are strained through 70 um strainers. Cells are sorted to purify the following population: CD45+CD56(−)CD3+E7TCRαb+CD19CAR+.

Cells are enumerated, and 2×10⁵ cells are grown in 200 ul OpTmiser medium with 300 IU/ml IL2, and 6×10⁵ CD3/CD28 stimulating Dynabeads (Thermo Fisher). Medium is changed every 2 days for a total of 2 weeks to allow cell expansion. Cells are replated to larger wells every 2 days maintaining a cell density of 1×10⁶ cells/ml.

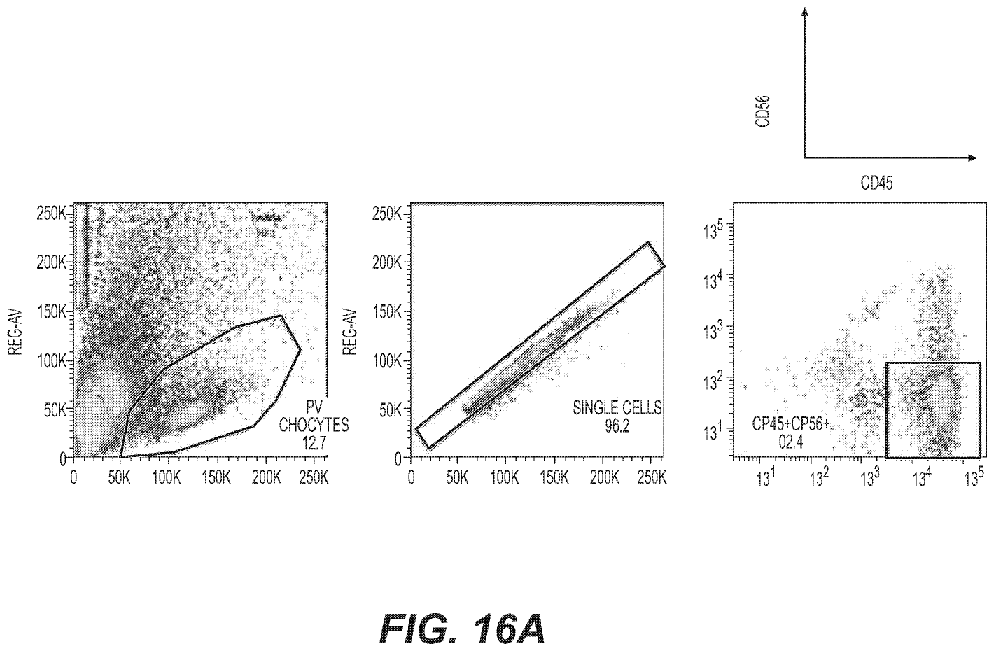

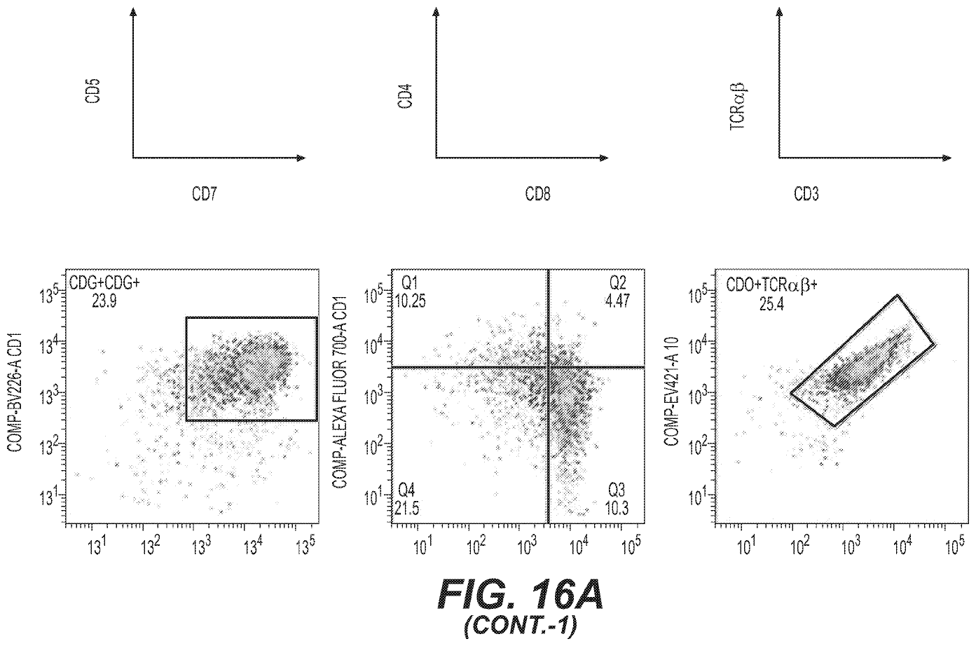

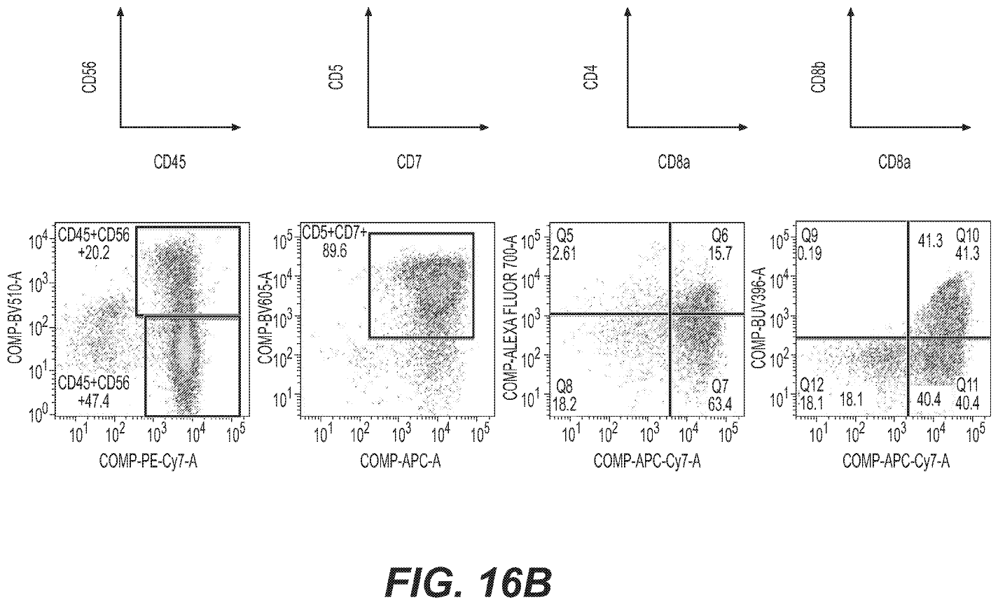

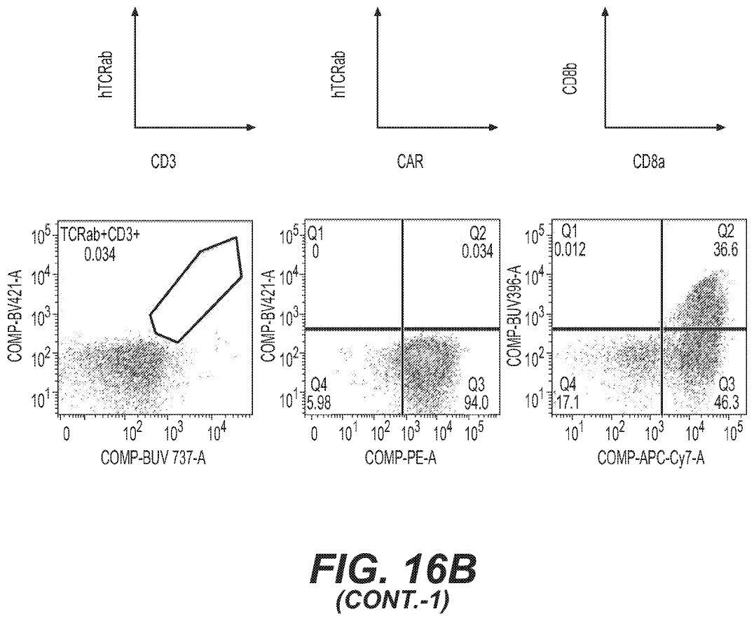

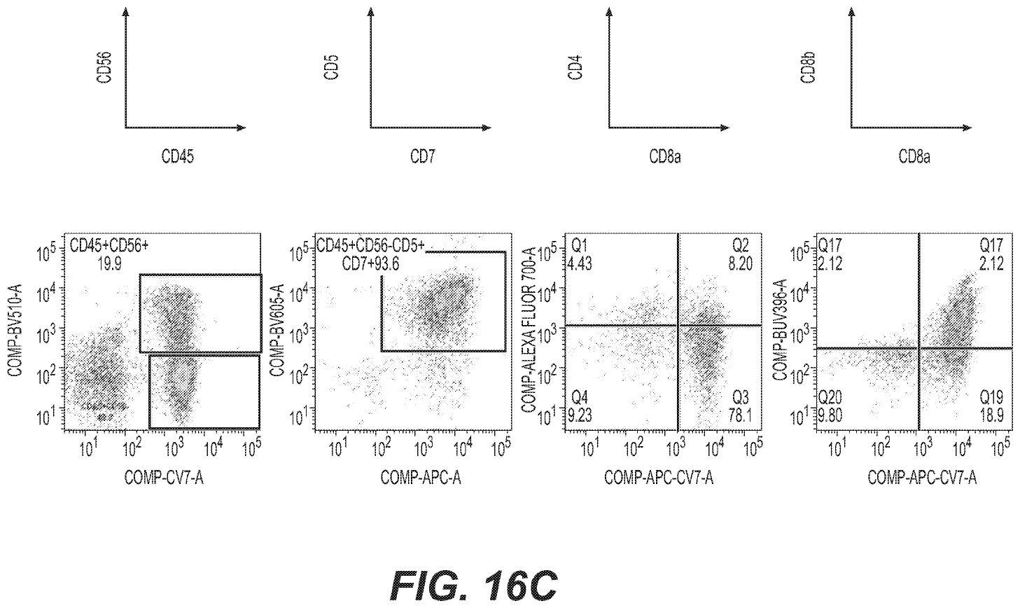

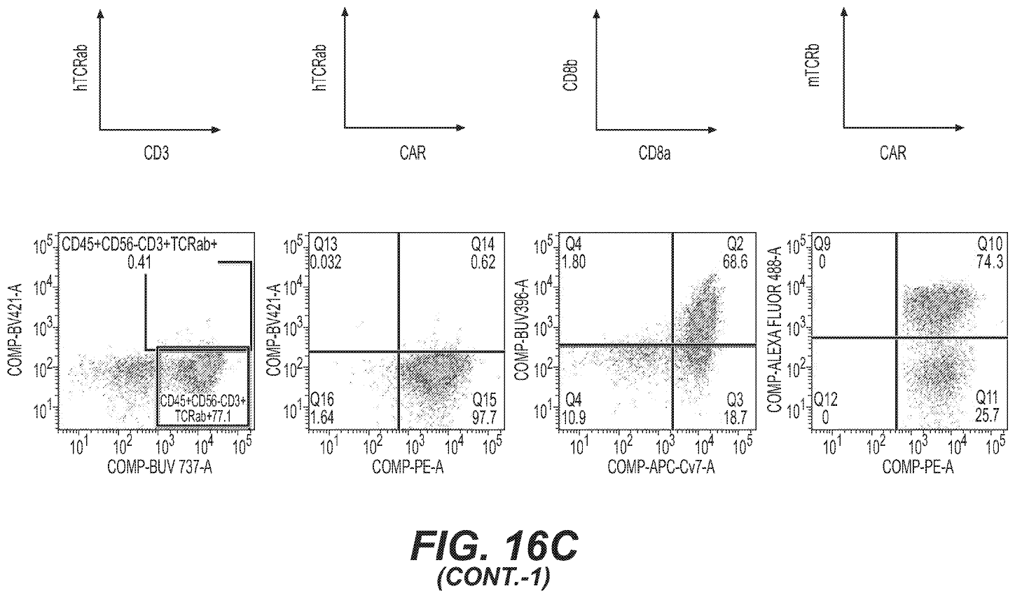

The cells were induced using the procedure described in this example. To evaluate the differentiation of iPSC cells to T-cells, FACS was used to analyze non-modified and modified (CAR-KI-TRAC) iPSC at weeks 3, 4, and 5, and stained with surface markers such as CD56, CD45, CD5, CD7, CD4, CD8α, CD8β, TCRαβ, CD3, or CD19CAR. Results of week 5 are shown in FIGS. 16A-16C.

### Example 3: Method of Controlling T-cell Differentiation Products

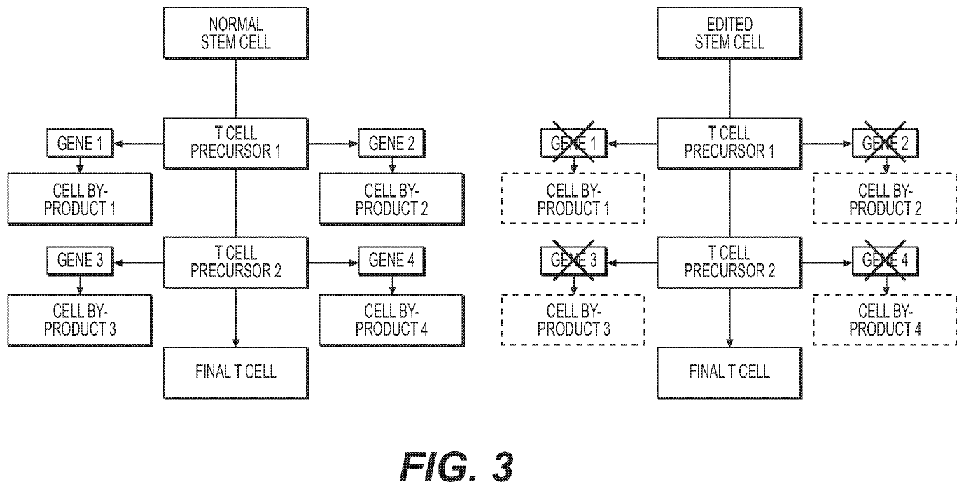

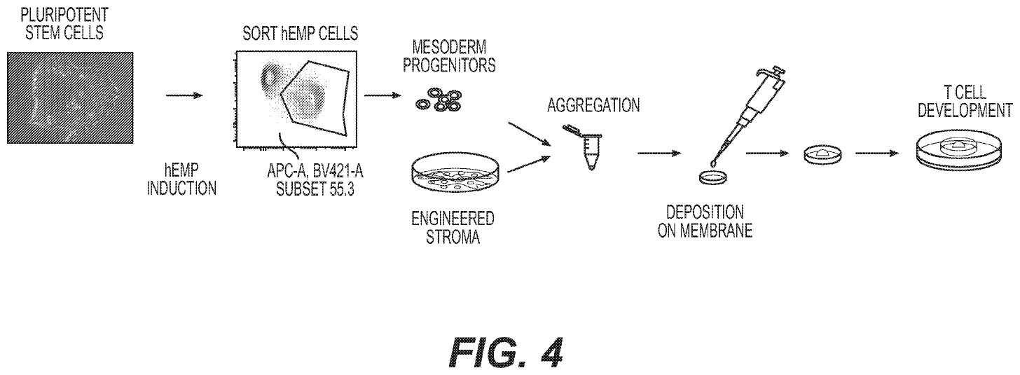

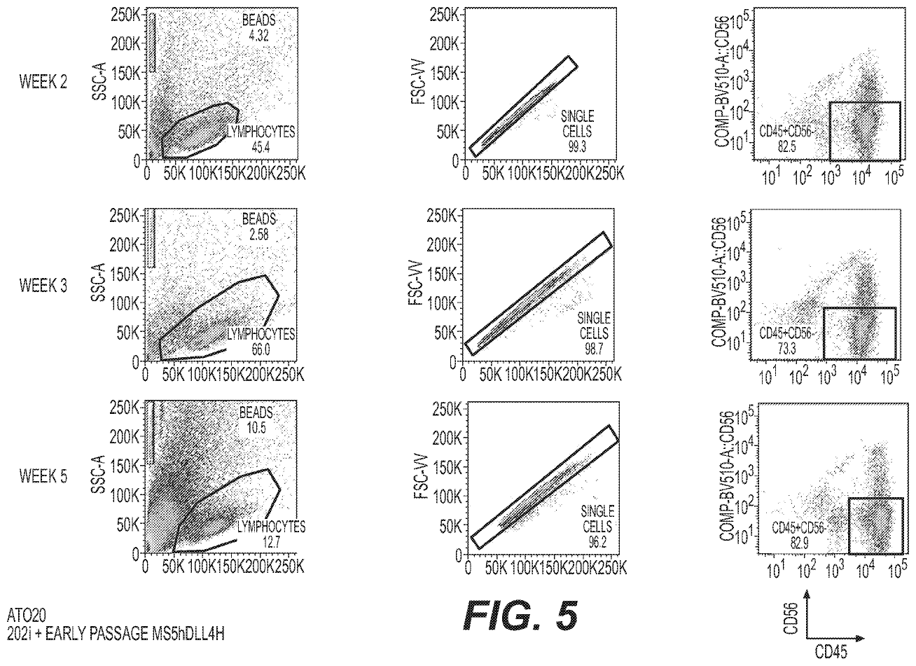

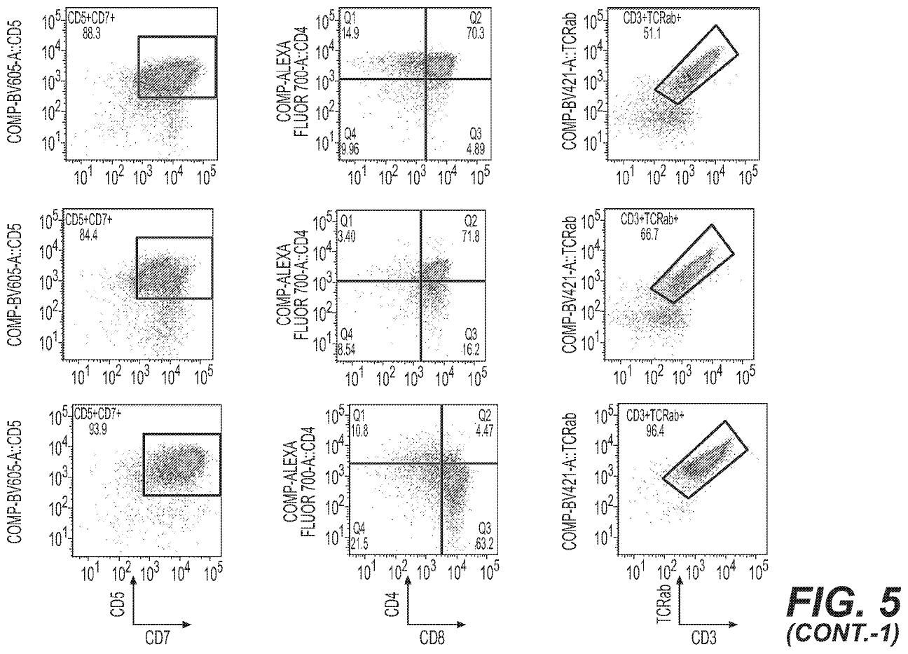

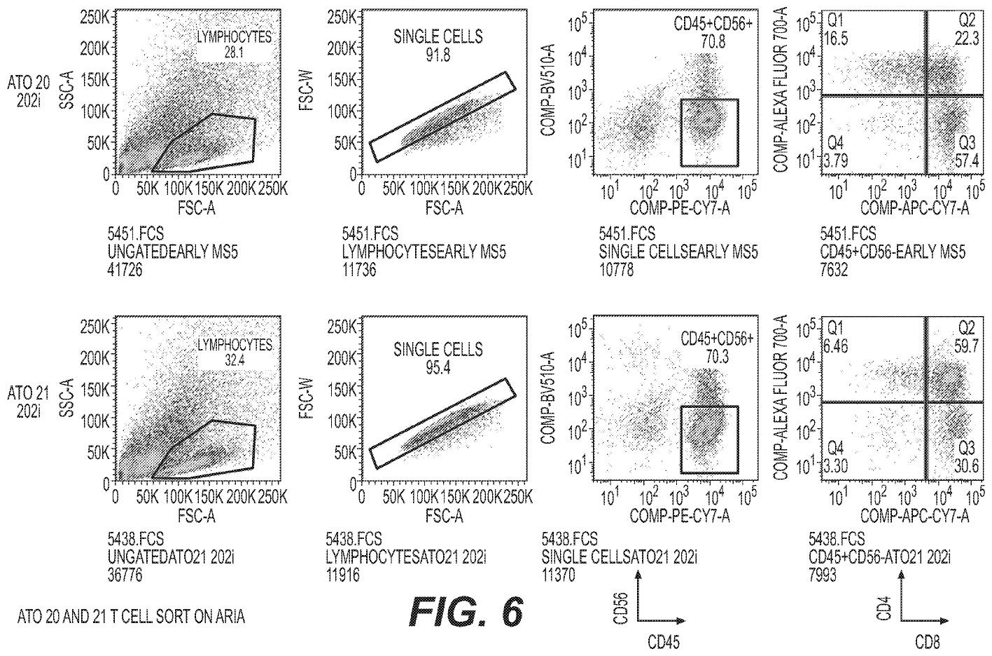

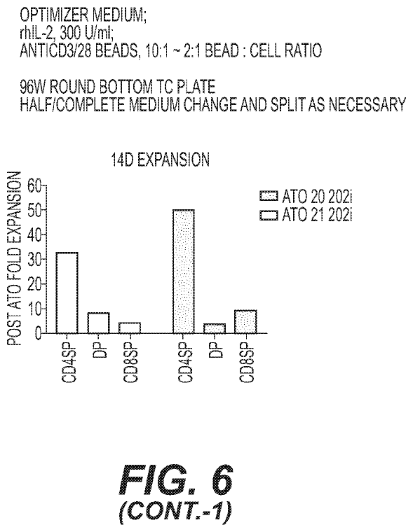

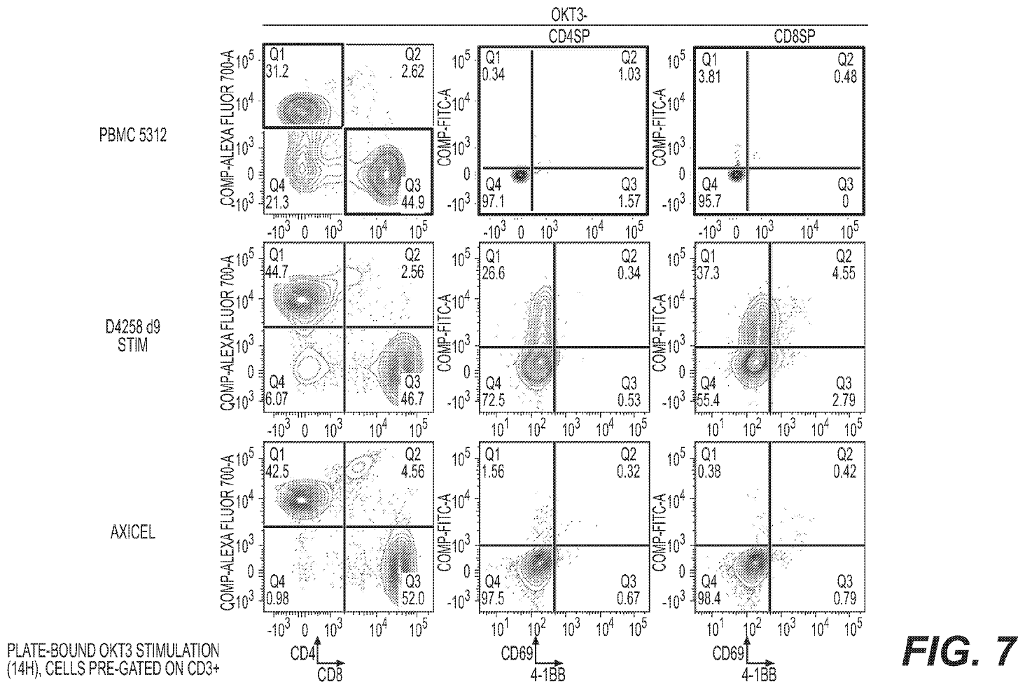

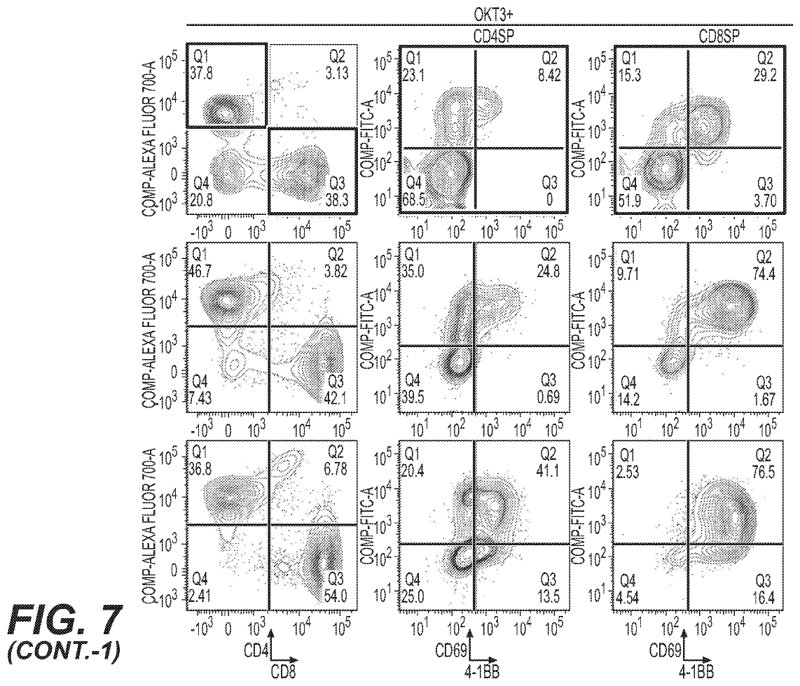

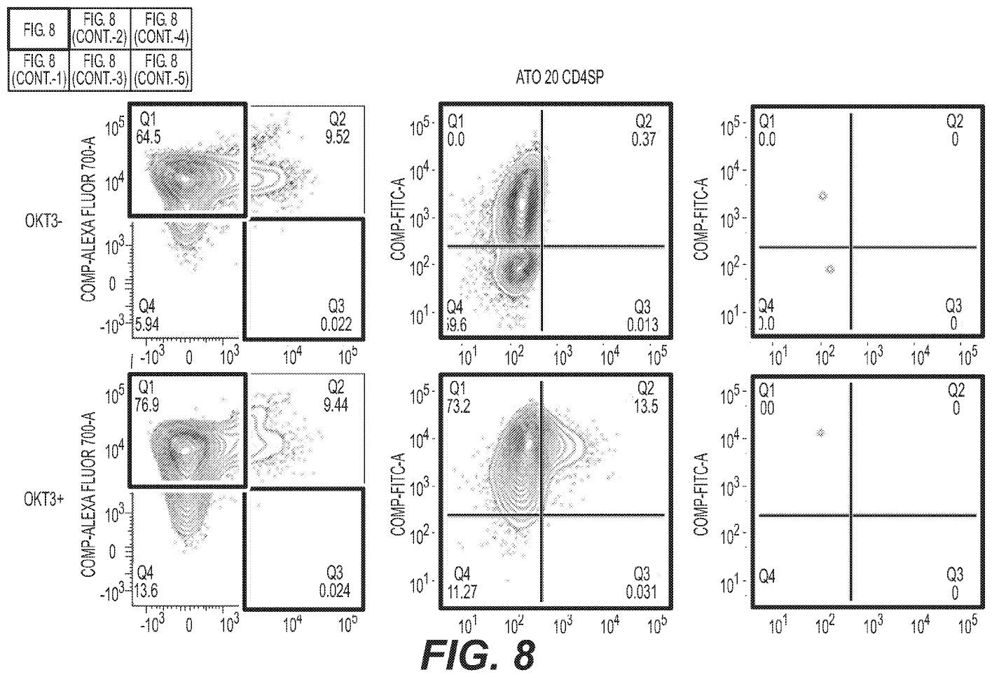

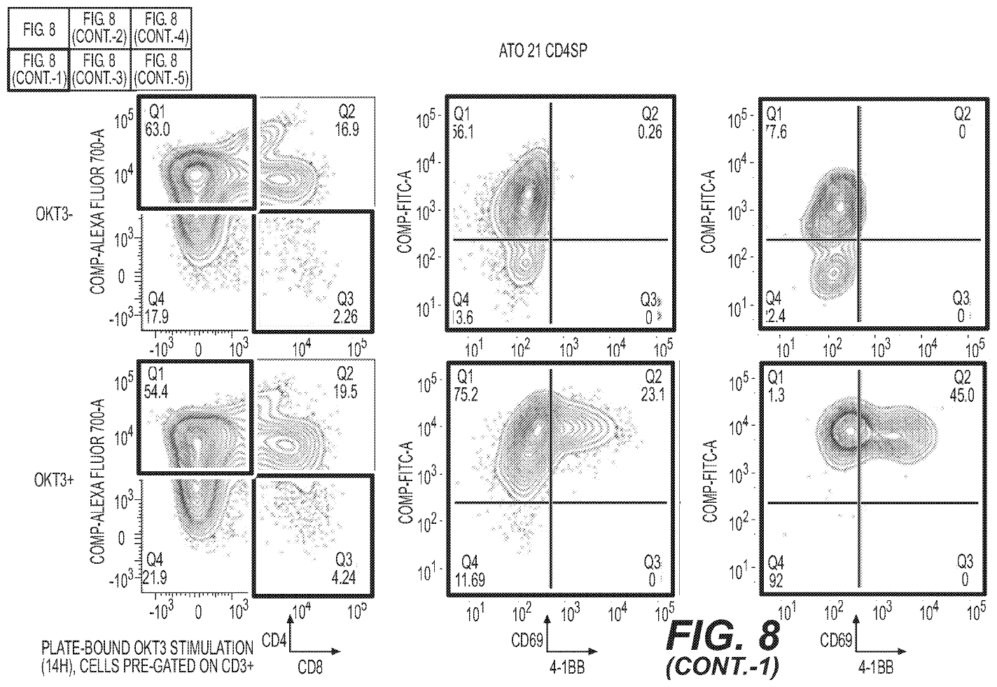

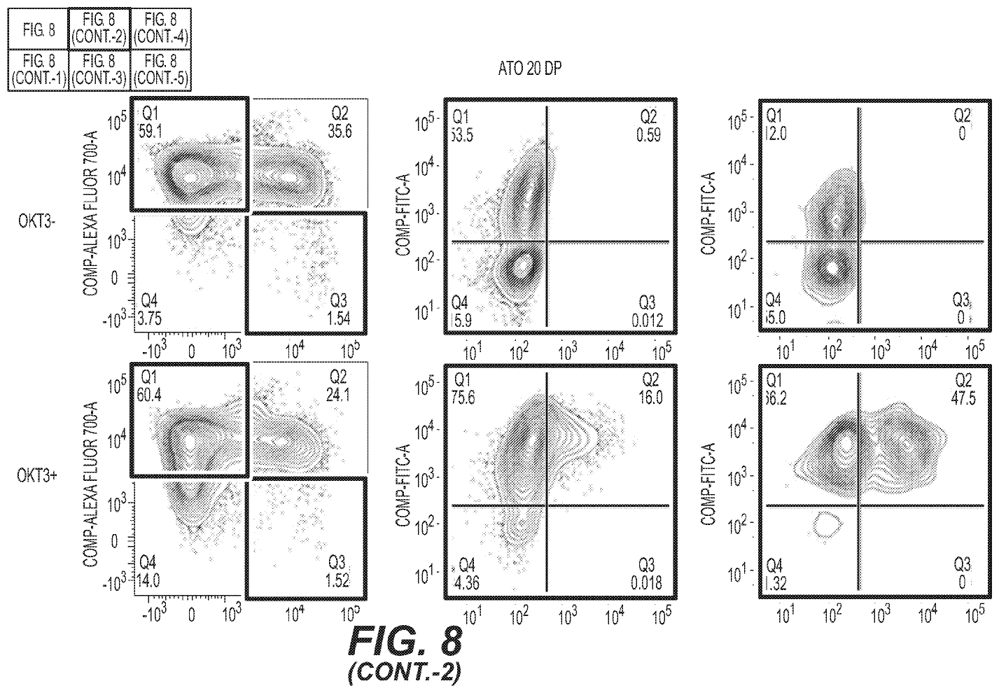

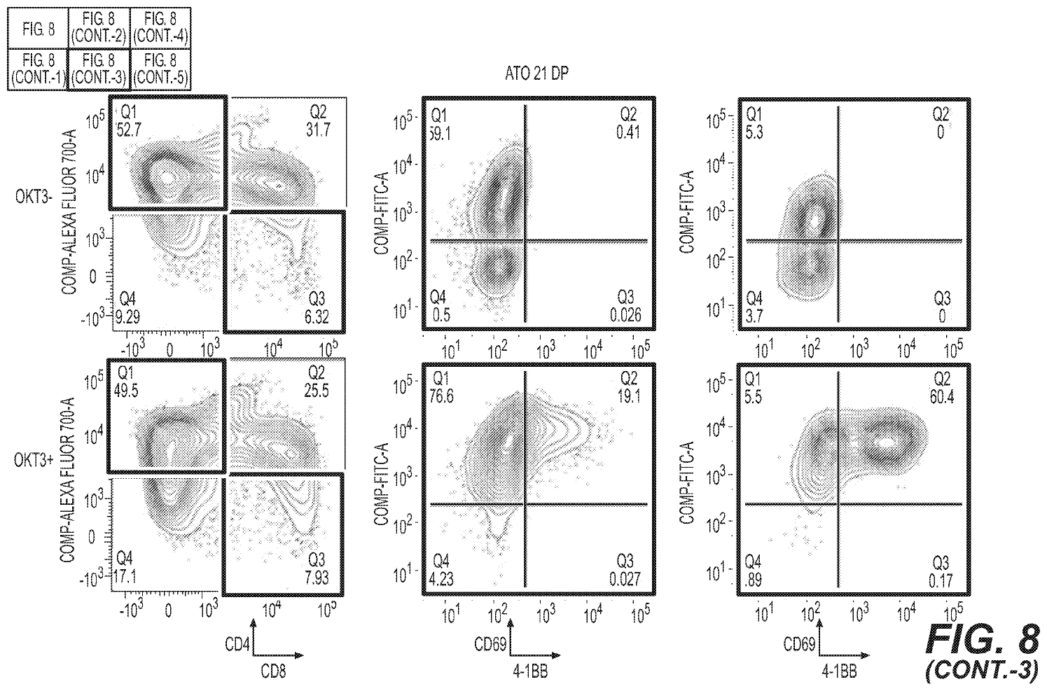

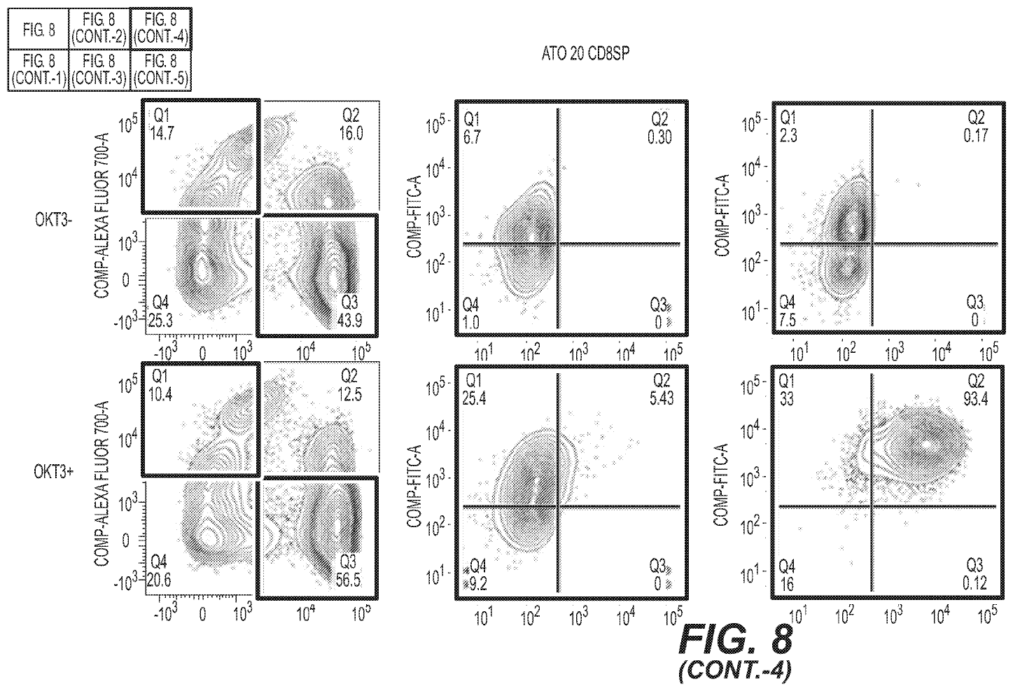

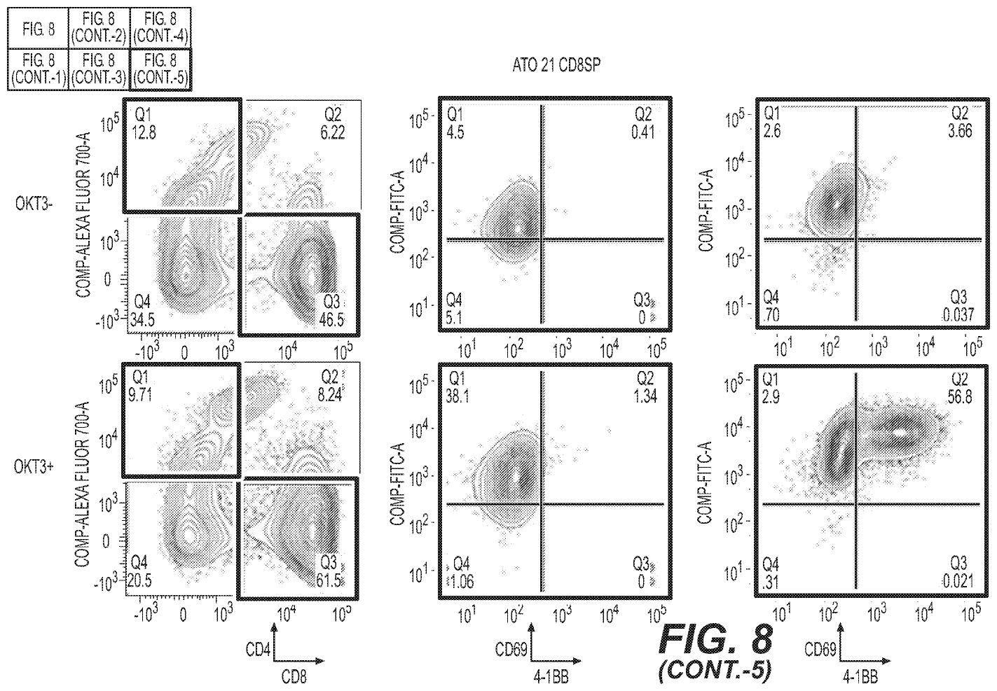

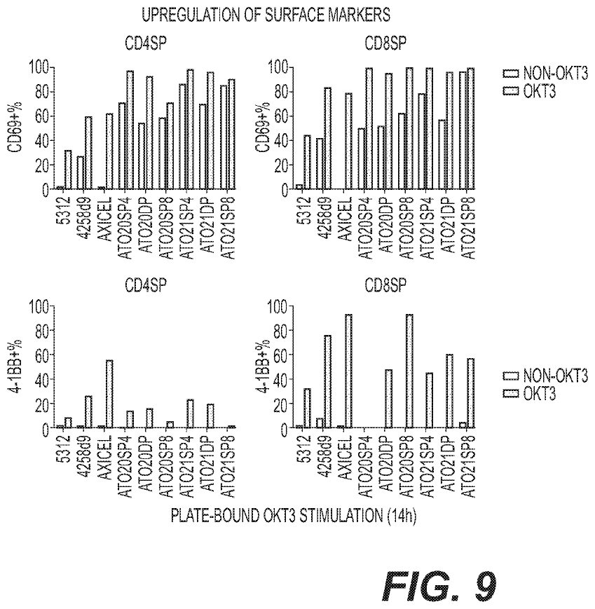

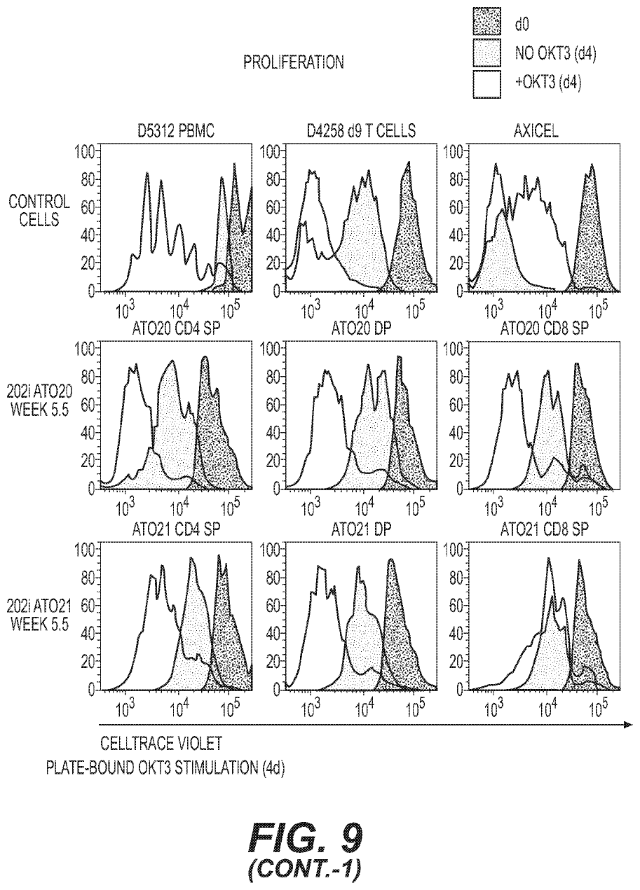

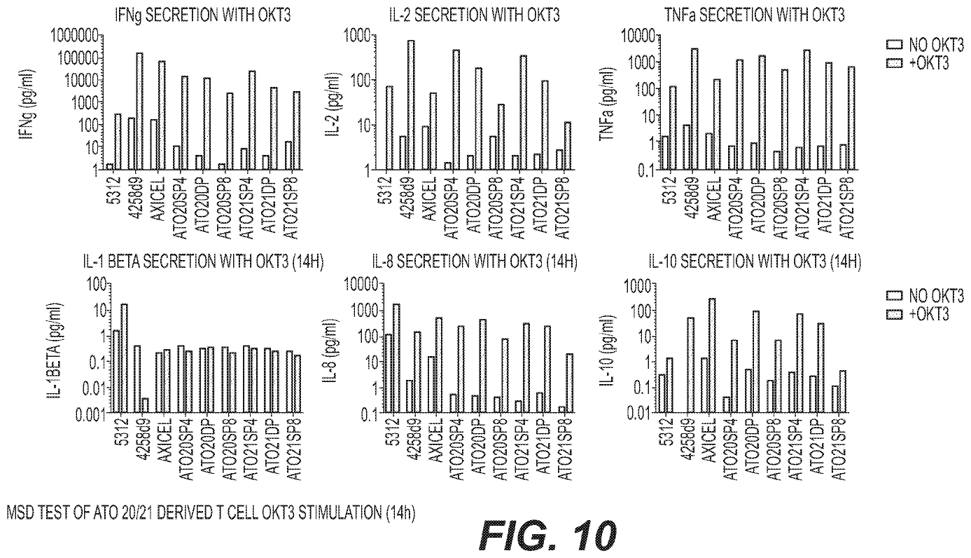

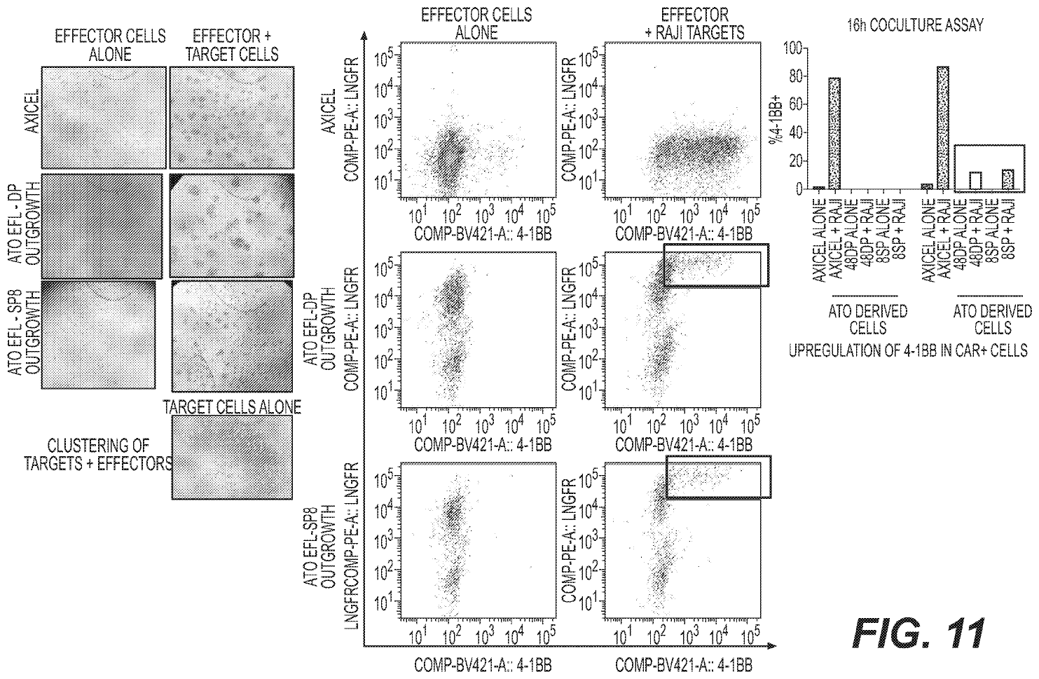

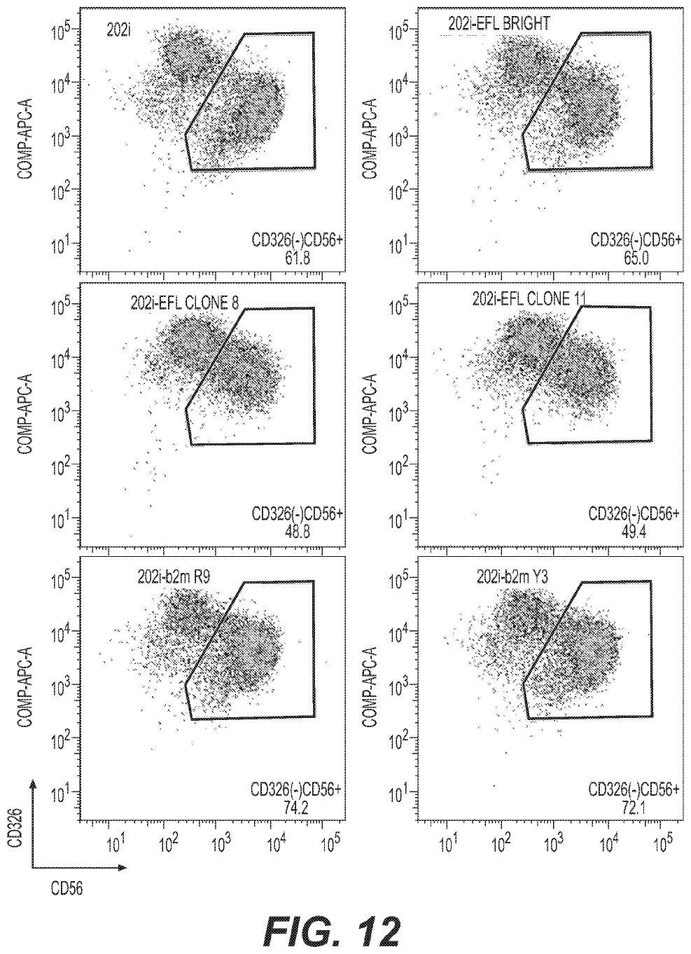

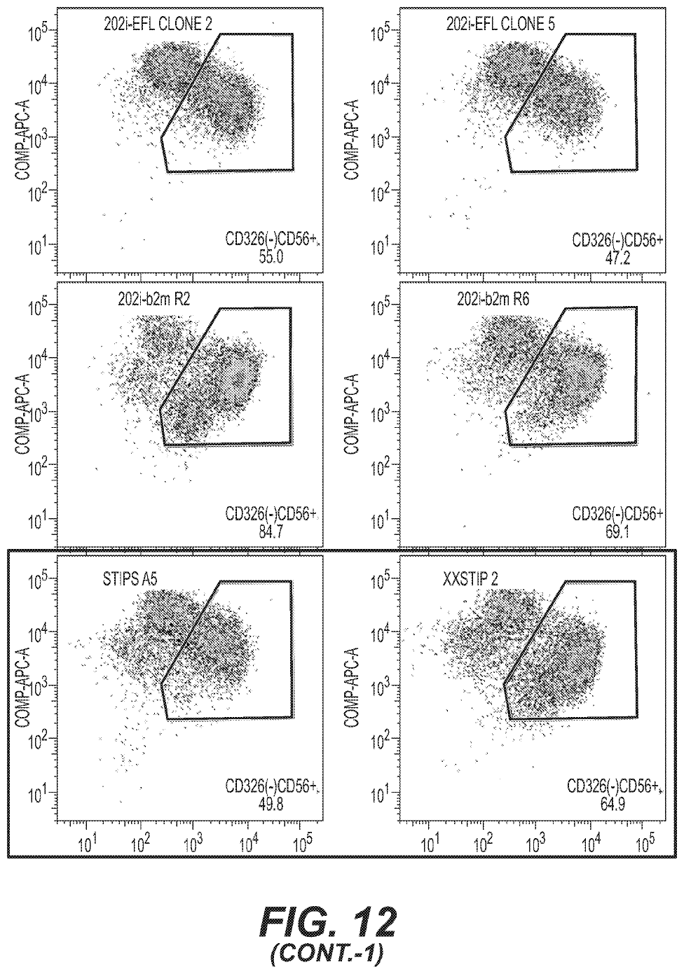

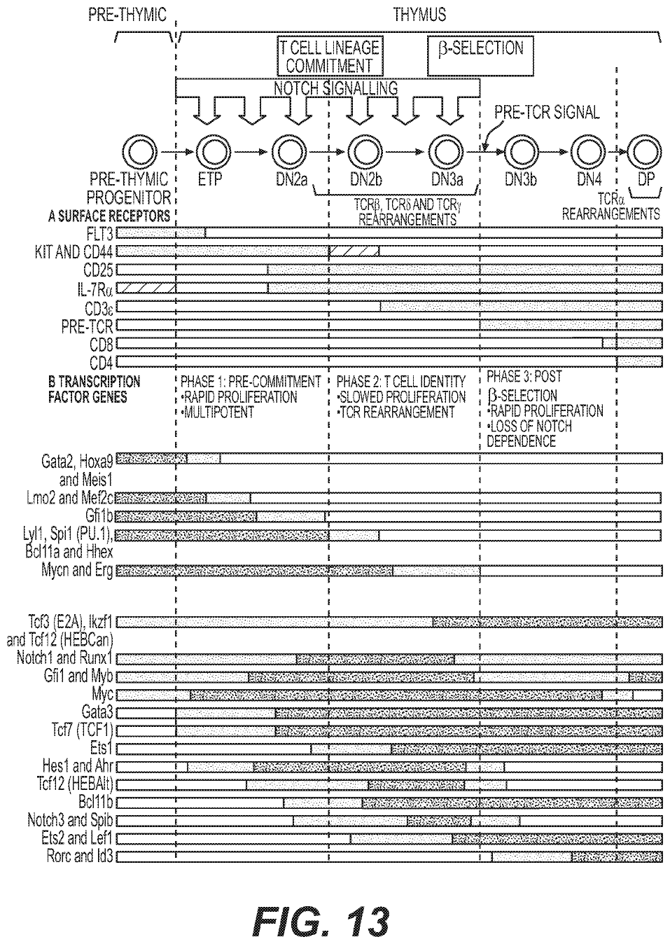

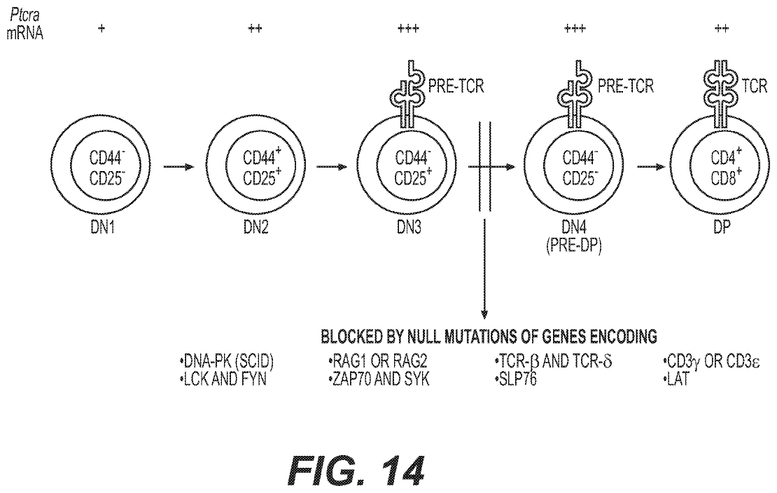

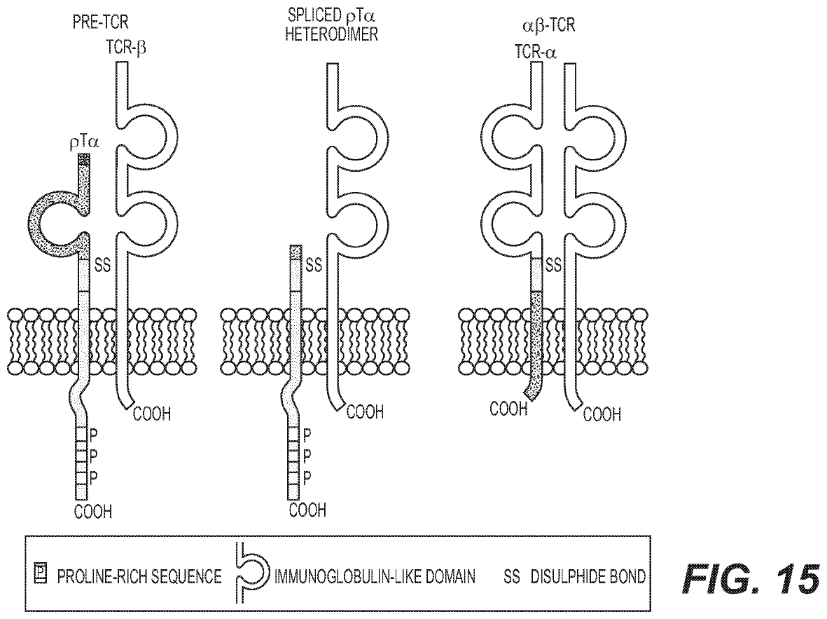

iPSCs are engineered to knock-out or modify certain critical master cell fate regulators, such as transcription factors, to impair or eliminate the generation of undesired cell by-products (FIG. 3).

Target genes are edited by knockout to eliminate the development of cell lineages as shown in Table 1.

TABLE 1
Target Genes to eliminate development of specific cell lineages

| Cell Lineage | Th1 | Th2 | Th9 | Th17 | Th22 | Tfh | Treg | NKT |
|---|---|---|---|---|---|---|---|---|
| Target Genes | T-BET | GATA3 | GATA3 | RORgt | AHR | Bcl-6 | FoxP3 | PLZF |
| | STAT1 | Stat5 | Smads | RORa | | MAF | Smad3 | |
| | STAT4 | Stat6 | Stat6 | Stat3 | | | Stat5 | |
| | STAT6 | DEC2 | PU.1 | | | | FOXO1 | |
| | RUNX3 | MAF | | | | | FOXO3 | |
| | | THPOK | | | | | GRAIL | |

### Example 4: Generating Engineered pTa Positive Stem Cells

This example illustrates preparation of engineered pTa positive stem cells.

Embryonic stem cells (ES) are modified to express exogenous pTa delivered by viral mediated delivery. Constructs are knocked into an endogenous gene locus taking advantage of innate gene regulatory elements, constitutive physiologic expression level, or contain a defined promoter. The defined promoter may be constitutively active or restricted to distinct stages of cell development and/or cell cycle, etc. ES cells expressing pTa with enriched pTa-TCRβ pairing are identified and isolated by known cell isolation techniques in the art.

### Example 5: Generating Engineered TCRα Knock Out Stem Cells

This example illustrates preparation of engineered TCRα knockout stem cells.

Induced pluripotent stem cells are engineered to knockout the endogenous TCRα using an engineered nuclease (e.g., CRISPR). iPS cells lacking surface expressed TCRα with enriched pTa-TCRβ pairing are identified and isolated by known cell isolation techniques in the art. The 179i and 202i cells were engineered or edited using the procedure described in Example 6. The pool populations and subsequent single clone were characterized using TIDE analysis (Table 5).

TABLE 5
Results of TIDE analysis in CRISPR edited 179i and 202i cells

| | | |
|---|---|---|
| indel size | percentage | p-value |

179i TRAC gRNP

| | | |
|---|---|---|
| 1 | 38.5 | 0 |
| 2 | 23.8 | 0 |
| −11 | 12.2 | 0 |
| −1 | 8.1 | 3.60E−255 |
| −5 | 4.2 | 1.60E−69 |
| −2 | 2.6 | 4.60E−29 |
| 0 | 2.4 | 3.70E−24 |
| −12 | 2.1 | 1.50E−19 |
| −3 | 1.4 | 3.80E−09 |
| −7 | 0.8 | 0.00042 |
| −14 | 0.6 | 0.0091 |

202i TRAC gRNP

| | | |
|---|---|---|
| 1 | 34.4 | 0 |
| −11 | 22.6 | 0 |
| 2 | 16 | 0 |
| −1 | 7.9 | 2.70E−150 |
| −5 | 5.9 | 1.60E−84 |
| 0 | 3.2 | 5.70E−26 |
| −2 | 2.3 | 9.00E−15 |
| −12 | 2 | 2.10E−11 |
| −3 | 0.9 | 0.0028 |

### Example 6: T Cell Differentiation From pTa Modified ES Cells

This example illustrates preparation of differentiated T cells from ES cells.

Isolated pTa modified cells described in Example 1 are stimulated to promote differentiation to T cells. Isolated pTa modified cells are provided and induced T cell differentiation in an artificial thymic organoid. The T cell lineage is selected by detecting expression of one or more biomarkers. In the present example, the T cell lineage of interest is cytotoxic CD8+ T cells, and are identified by the relative levels of surface expressed FLT3, KIT, CD25, CD44, IL-7Rα, CD3ε, Pre-TCR, CD8, and/or CD4.

1. A modified pluripotent stem cell engineered to eliminate endogenous TCR or HLA expression.

2. The cell of claim 1, comprising a deficient TCRα constant (TRAC) gene, a deficient TCRβ constant (TRBC) gene or a deficient beta 2 microglobulin (b2m) gene, optionally wherein the deficient gene is created by knockout.

3. The cell of claim 2, wherein the deficient gene is edited using CRISPR/Cas9, a zinc finger nuclease (ZFN), a TALEN, a MegaTAL, a meganuclease, Cpf1, homologous recombination, or a single stranded oligodeoxynucleotide (ssODN).

4. The cell of claim 1, comprising
an exogenous construct encoding a single chain HLA trimer, a single chain HLA trimer comprising an HLA linked to beta-2-microglobulin linked to a stabilizing peptide, optionally, wherein the HLA trimer is HLA-E, HLA-G, or a combination of HLA-E and HLA-G;
an exogenous construct encoding a chimeric antigen receptor (CAR) that targets a tumor antigen, optionally, wherein the tumor antigen is selected from a tumor-associated surface antigen, such as 5T4, alphafetoprotein (AFP), B7-1 (CD80), B7-2 (CD86), BCMA, B-human chorionic gonadotropin, CA-125, carcinoembryonic antigen (CEA), carcinoembryonic antigen (CEA), CD123, CD133, CD138, CD19, CD20, CD22, CD23, CD24, CD25, CD30, CD33, CD34, CD4, CD40, CD44, CD56, CD70, CD8, CLL-1, c-Met, CMV-specific antigen, CS-1, CSPG4, CTLA-4, DLL3, disialoganglioside GD2, ductal-epithelial mucine, EBV-specific antigen, EGFR variant III (EGFRvIII), ELF2M, endoglin, ephrin B2, epidermal growth factor receptor (EGFR), epithelial cell adhesion molecule (EpCAM), epithelial tumor antigen, ErbB2 (HER2/neu), fibroblast associated protein (fap), FLT3, folate binding protein, GD2, GD3, glioma-associated antigen, glycosphingolipids, gp36, HBV- specific antigen, HCV-specific antigen, HER1-HER2, HER2-HER3 in combination, HERV-K, high molecular weight-melanoma associated antigen (HMW-MAA), HIV-1 envelope glycoprotein gp41, HPV-specific antigen, human telomerase reverse transcriptase, IGFl receptor, IGF-II, IL-11Ralpha, IL-13R-a2, Influenza Virus-specific antigen; CD38, insulin growth factor (IGFl)-l, intestinal carboxyl esterase, kappa chain, LAGA-la, lambda chain, Lassa Virus-specific antigen, lectin-reactive AFP, lineage-specific or tissue specific antigen such as CD3, MAGE, MAGE-A1, major histocompatibility complex (MHC) molecule, major histocompatibility complex (MHC) molecule presenting a tumor-specific peptide epitope, M-CSF, melanoma-associated antigen, mesothelin, mesothelin, MN-CA IX, MUC-1, mut hsp70-2, mutated p53, mutated p53, mutated ras, neutrophil elastase, NKG2D, Nkp30, NY-ESO-1, p53, PAP, prostase, prostate specific antigen (PSA), prostate-carcinoma tumor antigen-1 (PCTA-1), prostate-specific antigen protein, STEAP1, STEAP2, PSMA, RAGE-1, ROR1, RU1, RU2 (AS), surface adhesion molecule, surviving and telomerase, TAG-72, the extra domain A (EDA) and extra domain B (EDB) of fibronectin and the Al domain of tenascin-C (TnC Al), thyroglobulin, tumor stromal antigens, vascular endothelial growth factor receptor-2 (VEGFR2), virus-specific surface antigen such as an HIV-specific antigen (such as HIV gpl20), as well as any derivate or variant of these surface markers;
an exogenous construct encoding a TCR, optionally, wherein the TCR is an alpha/beta TCR, gamma/delta TCR, a cancer or cancer associated antigen reactive TCR, a TCR that is reactive against murine or other non-human MHC, a class I or class II restricted TCR, an HPV recognizing TCR, a viral reactive TCR, an EBV TCR, a CMV TCR, or an influenza TCR, an HPV-16 E6 TCR, HPV-16 E7 TCR, or MAGEA3/A6 TCR or engineered variant, or TCR is derived from CD8, CD4, CD4/8 double positive, immature or developing T cell, Treg, NKT, or NK T cell; and/or
an exogenous construct encoding a suicide gene, wherein the suicide gene allows for the elimination of gene modified cells or is used as a PET reporter gene for non-invasive imaging, optionally, wherein the suicide gene is sr39TK, is a chemically induced caspase, dimerization induced by a small molecule/chemically induced dimerizer (CID), a selectable surface marker, or a selectable surface marker selected from CD19, CD20, CD34, EGFR or LNGFR.

5. A method of generating a modified pluripotent stem cell comprising:
(a) editing a gene locus to eliminate expression of endogenous TCR or block expression of donor HLA; and
(b) introducing an exogenous construct encoding a CAR, TCR, or HLA gene.

6. The method of claim 5, wherein the method further comprises a step of isolating a hematopoietic stem cell, an embryonic stem, or an induced pluripotent stem cell.

7. A method of generating a T cell lineage of interest comprising:
(a) providing a modified pluripotent stem cell of claim 1; and
(b) inducing T cell or T cell-like differentiation.

8. The method of claim 7, wherein T cell differentiation is induced using an artificial thymic organoid (ATO) system, notch agonist, OP9-DLL1, OP9-DLL4, fetal thymic organoid culture (FTOC), chemical induction, bone marrow/liver/thymus or other humanized mouse, embryoid body (EB).

9. The method of claim 7, wherein the T cell lineage is selected by detecting expression of one or more biomarkers, optionally, wherein the T cell lineage of interest is a CD8 single positive T cell, a CD4 single positive T cell, a CD4 CD8 double positive T cell, a double negative T cell, a CD3 positive cell, an NK cell, a proT cell, a pre-proT cell, a mesodermal progenitor, a B cell, a common lymphoid progenitor, a hematopoietic progenitor, a hematopoietic stem cell.

10. A method of generating a T cell lineage of interest, comprising:
(a) providing a modified pluripotent stem cell of claim 1;
(b) editing a gene encoding a cell fate regulator to promote, impair or eliminate the generation of a specific cell lineage; and
(c) inducing T cell differentiation.

11. The method of claim 10, wherein the cell fate regulator is a transcription factor, T-BET, STAT1, STAT4, STAT, RUNX3, GATA3, Stat5, Stat6, DEC2, MAF, THPOK, GATA3, Smads, Stat6, PU.1, RORgt, RORa, Stat3, AHR, Bcl-6, MAF, FoxP3, Smad3, Stat5, FOXO1, FOXO3, GRAIL, or PLZF.

12. The method of claim 10, wherein the specific lineage is Th1, Th2, Th9, Th17, Th22, Tfh, Treg, ILC, NK, or NKT.

13. A modified pluripotent stem cell with enriched or enhanced pairing between a pre-TCRα (pTa) protein and a TCRβ protein as compared to an unmodified control cell.

14. The modified pluripotent stem cell of claim 13, wherein the modified pluripotent stem cell comprises an exogenous construct encoding the pre-TCRα (pTa) protein, optionally, wherein the exogenous construct is a viral construct, an AAV construct, lentiviral construct, or retroviral construct.

15. The modified pluripotent stem cell of claim 13, wherein the modified pluripotent stem cell comprises a deficient TCRα gene, optionally, wherein the deficient TCRα gene is created by knockout using an engineered nuclease, TALEN, megaTAL, CRISPR, ZFN, knockout using homologous recombination, or antisense RNA.

16. The modified pluripotent stem cell of claim 13, wherein the modified pluripotent stem cell is substantially free of TCRα and TCRβ pairing.

17. The modified pluripotent stem cell of claim 13, wherein the modified pluripotent stem cell further comprises a chimeric antigen receptor (CAR), an exogenous TCR, and/or an antigen receptor.

18. A method of generating a modified pluripotent stem cell comprising a step of introducing an exogenous pre-TCRα (pTa) protein and/or creating a deficient TCRα gene.

19. A method of generating a T cell lineage of interest, comprising steps of providing a modified pluripotent stem cell of claim 1, and inducing T cell differentiation in an artificial thymic organoid.

20. A method of generating a T cell lineage of interest, comprising steps of providing a modified pluripotent stem cell of claim 1, and inducing T cell differentiation in the presence or absence of peptide:MHC, optionally, wherein the T cell lineage of interest is cytotoxic CD8+ T cells, helper CD4+ T cells, helper CD4+ T cells that are Th1/Th2/Th17 cells, regulatory T cells, intra epithelial lymphocyte (IEL), or mature alpha-beta or gamma-delta T cell.
